SEQUENCE LISTING (12 sequences)

SEQ ID NO: 1 - protein, 153 aa, Homo sapiens
MVATKTFALLLLSLFLAVGLGEKKEGHFSALPSLPVGSHAKVSSPQPRGPRYAEGTFISDYSIAMDKIHQQDFVNWLLAQKGKKNDWKHNITQREARALELASQANRKEEEAVEPQSSPAKNPSDEDLLRDLLIQELLACLLDQTNLCRLRSR

SEQ ID NO: 2 - protein, 32 aa, synthetic construct
  SITE 21: note = Palmitoyl-glutamic acid-hydroxyl attached to lysine side chain
HAEGTFTSDVSSYLDEGQAAKEPIAWLVRGRG

SEQ ID NO: 3 - protein, 39 aa, synthetic construct
HGEGTFTSDLSKQMEEEAVRLFIEWLKNGGPSSGAPPPS

SEQ ID NO: 4 - protein, 44 aa, synthetic construct
HGEGTFTSDLSKQMEEEAVRLFIEWLKNGGPSSGAPPSKKKKKK

SEQ ID NO: 5 - protein, 645 aa, synthetic construct
HGEGTFTSDVSSYLEGQAAKEFIAWLVKGRHGEGTFTSDVSSYLEGQAAKEFIAWLVKGRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 6 - protein, 275 aa, synthetic construct
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 7 - protein, 30 aa, synthetic construct
  SITE 2: note = Alpha-aminoisobutyric acid
  SITE 29: note = Alpha-aminoisobutyric acid
HXEGTFTSDVSSYLEGQAAKEFIAWLVKXR

SEQ ID NO: 8 - protein, 192 aa, Homo sapiens
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 9 - protein, 217 aa, Homo sapiens
MATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 10 - protein, 191 aa, synthetic construct [sequence omitted]

SEQ ID NO: 11 - protein, 217 aa, Homo sapiens
MAPGSRTSLLLAFALLCLPWLQEAGAVQTVPLSRLFDHAMLQAHRAHQLAIDTYQEFEETYIPKDQKYSFLHDSQTSFCFSDSIPTPSNMEETQQKSNLELLRISLLLIESWLEPVRFLRSMFANNLVYDTSDSDDYHLLKDLEEGIQTLMGRLEDGSRRTGQILKQTYSKFDTNSHNHDALLKNYGLLYCFRKDMDKVETFLRMVQCRSVEGSCGF

SEQ ID NO: 12 - protein, 30 aa, synthetic construct
  SITE 5: note = D-allo-threonine
  SITE 7: note = D-allo-threonine
  SITE 18: note = D-Alanine
  SITE 19: note = D-Alanine
  SITE 23: note = D-alloisolucene
  SITE 24: note = D-Alanine
HGEGXFXSDVSSYLEGQAAKEFXAWLVKGR

(19) United States
(12) Patent Application Publication
(10) Pub. No.: US 2024/0358650 A1
(43) Pub. Date: Oct. 31, 2024

(54) INHALABLE HORMONE RECEPTOR AGONIST FORMULATIONS

(71) Applicant: Pinata Holdings Inc., Northvale, NJ (US)

(72) Inventors: Michael OGBURN, Lake Forest, CA (US); Christopher PRICE, Monroe Township, NJ (US)

(21) Appl. No.: 18/751,870

(22) Filed: Jun. 24, 2024

Related U.S. Application Data
(63) Continuation of application No. PCT/US2023/081775, filed on Nov. 30, 2023.
(60) Provisional application No. 63/429,305, filed on Dec. 1, 2022.

(51) Int. Cl. A61K 9/50; A61K 9/48; A61K 38/26

(52) U.S. Cl. CPC A61K 9/5042; A61K 9/4808; A61K 9/4858; A61K 38/26

(57) ABSTRACT

Provided herein are pharmaceutical compositions, kits comprising pharmaceutical compositions, methods of treating disease, and methods of making compositions and kits described herein. The pharmaceutical compositions described herein are powdery pharmaceutical compositions. The powdery pharmaceutical compositions may be administered by an inhaler device described herein. The powdery pharmaceutical compositions may be administered by gummies described herein.

### CROSS-REFERENCE

This application is a continuation of PCT Application No. PCT/US2023/081775, filed Nov. 30, 2023, which claims the benefit of U.S. Provisional Application No. 63/429,305, filed Dec. 1, 2022, the disclosures of which are incorporated herein by reference in their entirety.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### SEQUENCE LISTING

The instant application contains a Sequence Listing which has been submitted electronically in ST.26 xml format and is hereby incorporated by reference in its entirety. Said xml copy, created on Jun. 10, 2024, is named 199238-711302_SL.xml and is 16,764 bytes in size.

### SUMMARY

Disclosed herein are powdery pharmaceutical compositions, comprising: a) particles of a pharmaceutically acceptable excipient; and b) a plurality of spray dried particles. In some embodiments, each particle of the plurality of spray dried particles can be substantially encapsulated in a coating material. In some embodiments, the plurality of spray dried particles substantially encapsulated in the coating material can comprise: an agonist selected from the group consisting of: a steroid or a pharmaceutically acceptable salt thereof, a human growth hormone receptor agonist or a pharmaceutical acceptable salt thereof, and a glucagon-like peptide 1 receptor agonist or a pharmaceutical acceptable salt thereof. In some embodiments, within the plurality of spray dried particles substantially encapsulated in the coating material at least a portion of the spray dried particles substantially encapsulated in the coating material can individually have a particle diameter ranging from about 1 micrometer to about 10 micrometers, as measured by a particle analyzer using laser diffraction. In some embodiments, the coating material can comprise trehalose, a hydroxypropyl methylcellulose (HPMC), a fumaryl diketopiperazine (FDKP), 1,2-distearoyl-sn-glycero-3-phosphocholine, a hydroxypropyl methylcellulose acetate succinate (HPMCAS), a povidone, a copovidone, a lactose, a phospholipid or any combination thereof. In some embodiments, the powdery pharmaceutical composition can further comprise a cannabinoid or a pharmaceutically acceptable salt thereof comprising tetrahydrocannabinol Delta-8, tetrahydrocannabinol Delta-9, tetrahydrocannabinol Delta-10, tetrahydrocannabinol Delta-11, tetrahydrocannabinol Delta-13, tetrahydrocannabivarin (THCV), tetrahydrocannabinolic acid (THCA), full spectrum THC, broad spectrum THC, or a pharmaceutically acceptable salt thereof. In some embodiments, the spray dried particles can comprise the steroid or a pharmaceutically acceptable salt thereof. In some embodiments, the steroid can be a testosterone or a pharmaceutically acceptable salt thereof. In some embodiments, the steroid can be an estrogen or a pharmaceutically acceptable salt thereof. In some embodiments, the steroid can be progesterone or a pharmaceutically acceptable salt thereof. In some embodiments, the spray dried particles can comprise the human growth hormone receptor agonist or a pharmaceutically acceptable salt thereof. In some embodiments, the spray dried particles can comprise the glucagon-like peptide 1 receptor agonist or a pharmaceutically acceptable salt thereof and the glucagon-like peptide 1 receptor agonist can comprise a semaglutide, a liraglutide, or a pharmaceutically acceptable salt of any of these. In some embodiments, the powdery pharmaceutical composition can be for inhaled use or for intranasal use. In some embodiments, the pharmaceutical composition can be in unit dose form. In some embodiments, at least a portion of the particles of the pharmaceutically acceptable excipient can individually have a particle diameter ranging from about 1 micrometers to about 200 micrometers, about 1 micrometer to about 10 micrometers, or about 50 micrometers to about 200 micrometers as measured by a particle analyzer using laser diffraction. In some embodiments, the particles can be admixed into a substantially homologous mixture. In some embodiments, the powdery pharmaceutical composition is contained within a capsule. In some embodiments, the capsule can be about one quarter to about one half, by volume, filled with the powdery pharmaceutical composition. In some embodiments, a weight-to-weight ratio of: a) the particles of the pharmaceutically acceptable excipient and b) the particles comprising the agonist or a pharmaceutically acceptable salt thereof, substantially encapsulated in a coating material, can range from about 1:1 (w/w) to about 10000:1 (w/w). In some embodiments, the weight-to-weight ratio of a) the particles of the pharmaceutically acceptable excipient and b) the particles of the agonist or the pharmaceutically acceptable salt thereof, can range from about 1:1 (w/w) to about 10:1 (w/w). In some embodiments, the portion of the capsule not containing the powdery pharmaceutical composition can comprise a gas that at least partially comprises an inert gas. In some embodiments, the capsule can comprise a hydroxypropylmethyl cellulose (HPMC) capsule. In some embodiments, the capsule can be size: 000, 00, 0, 1, 2, 3, or 4. In some embodiments, the capsule is size 3. In some embodiments, the powdery pharmaceutical composition can be contained within an inhaler unit. In some embodiments, the capsule can be contained in an inhaler unit. In some embodiments, the pharmaceutically acceptable excipient can comprise a carbohydrate, an alginate, povidone, a carbomer, a flavor, a natural gum, a silicone, an alcohol, a butter, a wax, a fatty acid, a preservative, a fumaryl diketopiperazine (FDKP), a pharmaceutically acceptable salt of any of these, or any combination thereof. In some embodiments, the pharmaceutically acceptable excipient can comprise the carbohydrate or the pharmaceutically acceptable salt thereof, and the carbohydrate or the pharmaceutically acceptable salt thereof can comprise a lactose, a microcrystalline cellulose, a cellulose, a mannitol, a sorbitol, a starch, a starch glycolate, a hydroxypropyl methylcellulose, a hydroxypropyl methylcellulose acetate succinate, a cyclodextrin, a maltodextrin, a croscarmellose sodium, a corn starch, a carrageenan, a sorbitol, a maltitol, a glucose, a pharmaceutically acceptable salt of any of these, or any combination thereof. In some embodiments, the pharmaceutically acceptable excipient thereof can comprise a lactose or a pharmaceutically acceptable salt thereof. In some embodiments, the lactose can comprise a milled lactose, a sieved lactose, a micronized lactose, a spray dried lactose, an at least substantially anhydrous lactose, a monohydrate lactose, a pharmaceutically acceptable salt thereof, or any combination thereof. In some embodiments, the powdery pharmaceutical composition described herein when stored in a sealed container placed in a room at 25° C. and a room atmosphere having about 50 percent relative humidity, retains at least about: 90% of the agonist thereof after 6 months, as measured by HPLC. In some embodiments, the agonist can be present in an amount ranging from about 0.001 mg to about 20 mg. In some embodiments, the agonist can be in the form of a pharmaceutically acceptable salt thereof and can be a hydrochloride salt, a bitartrate salt, or a borate salt. In some embodiments, the particles comprising the agonist can comprise a median diameter of less than about: 5 μm, 6 μm, 7 μm, 8 μm, 9 μm or 10 μm.

Also disclosed herein are kits comprising the powdery pharmaceutical compositions disclosed herein contained at least in part in a container.

Also disclosed herein are methods of treating a disease or condition in a subject in need thereof, comprising treating the disease or condition by administering, via inhalation, a first therapeutic comprising a therapeutically effective amount of the powdery pharmaceutical composition disclosed herein to the subject in need thereof. In some embodiments, the administering can be conducted one, twice, three, or four times per day. In some embodiments, the agonist can be the steroid or the pharmaceutically acceptable salt thereof. In some embodiments, the steroid or the pharmaceutically acceptable salt thereof can be a testosterone or a salt thereof. In some embodiments, the disease or condition can be selected from the group consisting of: an obesity, a low libido, an underdeveloped muscle mass, an underdeveloped secondary sex characteristic, a depression, an underdeveloped body hair growth, an underdeveloped voice deepening, an underdeveloped tendon, an underdeveloped ligament, and a lack of spermatogenesis. In some embodiments, the steroid or the pharmaceutically acceptable salt thereof can be an estrogen or a salt thereof. In some embodiments, the powdery pharmaceutical composition can further comprise a cannabinoid or a pharmaceutically acceptable salt thereof comprising tetrahydrocannabinol (THC), cannabidiol (CBD), cannabinol (CBN), cannabigerol (CBG), cannabichromene (CBC), cannabicyclol (CBL), cannabivarin (CBV), cannabidivarin (CBDV), cannabichromevarin (CBCV), cannabigerovarin (CBGV), cannabigerol monomethyl ether (CBGM), and cannabidiolic acid (CBDA), cannabielsoin (CBE), cannabicitran (CBT), 10-Oxo-delta-6a-tetrahydrocannabinol (OTHC), cannabichromanon, cannabifuran, cannabiglendol, cannabiripsol, cannbicitran, or any combination thereof. In some embodiments, the disease or condition can be selected from the group consisting of: a lack of female secondary sex characteristics, a low vaginal lubrication, an underdeveloped uterine growth, an unwanted bone reabsorption, a low bone formation, an undesirably low levels of body fat, thinning of a vaginal wall, an infertility, a regulation of ovulation, a regulation of menstruation, an endometriosis, a depression, an obsessive compulsive disorder, and an eating disorder. In some embodiments, the steroid or the pharmaceutically acceptable salt thereof can be a progesterone or a salt thereof. In some embodiments, the disease or condition can be selected from the list consisting of: a uterine cancer, a cervical cancer, an inability to prevent pregnancy, an inability to maintain a pregnancy, an unwanted preterm labor, an unwanted lactation during pregnancy, an insufficient breast development for breastfeeding, a skin aging, and a libido regulation. In some embodiments, the agonist can be the human growth hormone receptor agonist or the pharmaceutical acceptable salt thereof. In some embodiments, the disease or condition can be selected from the group consisting of: a low calcium retention, a low muscle mass, an obesity, a low internal organ growth, a high liver uptake of glucose, a low gluconeogenesis in the liver, a dysfunction of pancreatic islets, a poor immune system stimulation, a low cognitive function, a Turner syndrome, a chronic kidney failure, a Prader-Willi syndrome, an intrauterine growth restriction, a severe idiopathic short stature, a muscle mass wasting from AIDS, a low body density, a multiple sclerosis, an obesity, a fibromyalgia, a heart failure, a Chron's disease, and an ulcerative colitis. In some embodiments, the agonist can be the glucagon-like peptide 1 receptor agonist or the pharmaceutical acceptable salt thereof. In some embodiments, the disease or condition can be selected from the list of: a type 1 diabetes, a type 2 diabetes, a type 3c diabetes (pancreatogenic diabetes), an obesity, an overweight, a coronary artery disease (CAD), a dementia, a coronary heart disease (CHD), an ischemic heart disease (IHD), and a myocardial ischemia. In some embodiments, the powdery pharmaceutical composition can be administered as needed, or for about: one day, two days, three days, four days, five days, six days, a week, two weeks, three weeks, a month, two months, three months, four months, five months, six months, seven months, eight months, nine months, ten months, eleven months, a year, or chronically. In some embodiments, a second therapeutic or a pharmaceutically acceptable salt thereof can be administered. In some embodiments, the second therapeutic or the pharmaceutically acceptable salt thereof can be administered concurrently or consecutively. In some embodiments, the second therapeutic or the pharmaceutically acceptable salt thereof can be comprised in the powdery pharmaceutical formulation. In some embodiments, the first therapeutic can comprise the steroid or the pharmaceutically acceptable salt thereof and the second therapeutic can comprise the human growth hormone receptor agonist or the pharmaceutically acceptable salt thereof. In some embodiments, the subject can be diagnosed with the disease or condition. In some embodiments, the diagnosing can comprise employing an in vitro diagnostic. In some embodiments, the in vitro diagnostic can be a companion diagnostic. In some embodiments, the inhalation can be oral inhalation, intranasal administration, or any combination thereof.

Also disclosed herein are powdery pharmaceutical compositions, comprising: particles of a pharmaceutically acceptable carrier; and plurality of spray dried particles. In some embodiments, each particle of the plurality of spray dried particles can be substantially unencapsulated. In some embodiments, the plurality of spray dried particles substantially unencapsulated can comprise: an agonist selected from the group consisting of: a steroid or a pharmaceutically acceptable salt thereof, a human growth hormone receptor agonist or a pharmaceutical acceptable salt thereof, and a glucagon-like peptide 1 receptor agonist or a pharmaceutical acceptable salt thereof. In some embodiments, within the plurality of spray dried particles, the plurality of spray dried particles can comprise a mass median aerodynamic diameter of less than 5 μm and a fine particle fraction of at least about 40% upon aerosolization. In some embodiments, the pharmaceutically acceptable carrier can comprise trehalose, a hydroxypropyl methylcellulose (HPMC), a fumaryl diketopiperazine (FDKP), a 1,2-distearoyl-sn-glycero-3-phosphocholine, a hydroxypropyl methylcellulose acetate succinate (HPMCAS), a povidone, a copovidone, a lactose, a phospholipid, or any combination thereof. In some embodiments, the powdery pharmaceutical composition can comprise at least about 2% to about 20% of the agonist. In some embodiments, the agonist can be the steroid or the pharmaceutically acceptable salt thereof. In some embodiments, the steroid or the pharmaceutically acceptable salt thereof can be a testosterone or a pharmaceutically acceptable salt thereof. In some embodiments, the steroid or the pharmaceutically acceptable salt thereof can be an estrogen or a pharmaceutically acceptable salt thereof. In some embodiments, the steroid or the pharmaceutically acceptable salt thereof can be a progesterone or a pharmaceutically acceptable salt thereof. In some embodiments, the agonist can be the human growth hormone receptor agonist or the pharmaceutically acceptable salt thereof. In some embodiments, the agonist can be the glucagon-like peptide 1 receptor agonist or the pharmaceutically acceptable salt thereof. In some embodiments, the powdery pharmaceutical composition can comprise the agonist in an amount from about 3 mg to about 30 mg. In some embodiments, the powdery pharmaceutical composition can comprise the agonist in an amount of at least about 0.25 mg. In some embodiments, the powdery pharmaceutical composition can be packaged to have a delivered dose of at least about 0.075 mg.

### BRIEF DESCRIPTION OF THE DRAWINGS

FIG. 1A shows a dry powder inhaler device for delivery of a powdery pharmaceutical composition to the lung alveolar and FIG. 1B shows a nasal inhaled device for intranasal delivery of a powdery pharmaceutical composition to the lung alveolar.

FIG. 2 shows the method of use for the dry powder inhaler device for delivery of a powdery pharmaceutical composition.

FIG. 3 shows a spray drying manufacturing system comprising a closed spray dryer container which receives a solution comprising a drug dissolved or mixed in a suitable solvent. The system generates solid particles from the solution comprising the drug.

FIG. 4 shows a protective cap for a dry powder inhaler device.

FIG. 5 shows a rotatable mouthpiece of a dry powder inhaler device.

FIG. 6 shows a lower base chamber receptacle of a dry powder inhaler device.

FIG. 7 shows a lateral button operably connected to a sharp surface for use in a dry powder inhaler device for piecing a capsule containing a dry powdery pharmaceutical composition.

FIG. 8 shows a base plate of a dry powder inhaler device.

FIG. 9 shows a dry powder inhaler device for delivery of a powdery pharmaceutical composition to the lung alveolar.

FIG. 10 shows a spray drying manufacturing system comprising a closed spray drying chamber which receives a solution comprising an active ingredient, an encapsulating polymer, and a suitable solvent.

### DETAILED DESCRIPTION

### Overview

Delivering pharmaceutical compositions through oral ingestion of capsules or tablets may take a long time to dissolve and reach the blood stream. The absorption through stomach may take longer if fatty foods are eaten prior to ingestion of the capsule or tablet, further slowing down the process. By spray drying the pharmaceutical compositions and introducing them into the lungs via inhalation, the time needed for the pharmaceutical to reach the blood stream may be significantly reduced. In addition, the dosing level may also be reduced as compared to the oral tablet or capsule equivalent.

Provided herein are methods and pharmaceutical formulations for the treatment of conditions characterized by low androgen levels such as an obesity, a low libido, an underdeveloped muscle mass, an underdeveloped secondary sex characteristic, a depression, an underdeveloped body hear growth, an underdeveloped voice deepening, an underdeveloped tendon, an underdeveloped ligament, lack of spermatogenesis and the like.

Also provided herein are methods and pharmaceutical formulations for the treatment of conditions characterized by low estrogen levels such as regulation of the female reproductive system, a lack of female secondary sex characteristics, low vaginal lubrication, an underdeveloped uterine growth, an unwanted bone reabsorption, a low bone formation, an undesirably low level of body fat, a thinning vaginal wall, an infertility, a regulation of ovulation, a regulation of menstruation, an endometriosis, a depression, an obsessive compulsive disorder, an eating disorder, and a low libido which can be found in both men and women and the like.

Also provided herein are methods and pharmaceutical formulations for the treatment of conditions characterized by low progesterone levels such as an uterine cancer, a cervical cancer, an inability to prevent pregnancy, an inability to maintain a pregnancy, an unwanted preterm labor, an unwanted lactation during pregnancy, an insufficient breast development for breastfeeding, a skin aging, libido regulation, and the like.

Also provided herein are agonists that bind to steroid hormone receptors. The steroid hormone receptors may be androgen receptors, estrogen receptors, and progesterone receptors.

Also provided herein are agonists that bind to estrogen steroid receptors. Estrogen receptors may include nuclear estrogen receptors such as Era and Erp as well as membrane estrogen receptors such as GPR30, ER-X, Gq-mER. Also provided herein are agonists that bind to a progesterone steroid receptor such as NR3C3 and agonists that bind to an androgen steroid receptor such as NR3C4.

Also disclosed herein among other things is the production pharmaceutical compositions. In some cases, the pharmaceutical compositions can be used for the delivery of steroid receptor agonists for the treatments listed above. In some instances, a steroid receptor agonist is a drug that is capable of binding to a steroid receptor. The drug may be delivered as a dry powder drug utilizing inhalation or intranasal administration as the route of administration. In some cases, the drug may be microencapsulated. In some cases, the drug may not be microencapsulated.

Also provided herein are powdery compositions comprising particles of at least partially encapsulated metformin. In some cases, the at least partially encapsulated metformin can be incorporated into a gummy and administered to a subject to treat a disease or condition such as type 2 diabetes. In some cases, the gummy is at least partially encapsulated with an enteric coating such as methyl methacrylate.

Also provided herein are pharmaceutical compositions, kits comprising pharmaceutical compositions, methods of treating and preventing disease, and methods of making compositions and kits described herein. Pharmaceutical compositions described herein may be produced employing various methods to synthesize, manipulate, and administer particles. In some aspects, the pharmaceutical compositions described herein are powdery pharmaceutical compositions.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various aspects may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a,” “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement and include determining if an element may be present or not (for example, detection). These terms may include quantitative, qualitative or quantitative, and qualitative determinations. Assessing may be alternatively relative or absolute. “Detecting the presence of” includes determining the amount of something present, as well as determining whether it may be present or absent.

The terms “subject,” “individual,” “host”, or “patient” are often used interchangeably herein. A “subject” may be a biological entity containing expressed genetic materials. The biological entity may be a plant, animal, or microorganism, including, for example, bacteria, viruses, fungi, and protozoa. The subject may be tissues, cells and their progeny of a biological entity obtained in vivo or cultured in vitro. The subject may be a mammal. The mammal may be a human. The subject may be diagnosed or suspected of being at high risk for a disease. In some cases, the subject may not be necessarily diagnosed or suspected of being at high risk for the disease.

A “Homology” or “identity” or “similarity” can refer to sequence similarity between two peptides or between two nucleic acid molecules. Homology can be determined by comparing a position in each sequence which can be aligned for purposes of comparison. When a position in the compared sequence can be occupied by the same base or amino acid, then the molecules can be homologous at that position. A degree of homology between sequences can be a function of the number of matching or homologous positions shared by the sequences. An “unrelated” or “non-homologous” sequence shares less than 40% identity, or alternatively less than 25% identity, with one of the sequences of the disclosure. Sequence homology can refer to a % identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein (which can correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity can be calculated over the full-length of the reference sequence and that gaps in sequence homology of up to 5% of the total reference sequence can be allowed.

In some cases, the identity between a reference sequence (query sequence) and a subject sequence, also referred to as a global sequence alignment, can be determined using the FASTDB computer program-based on the algorithm of Brutlag et al. (Comp. App. Biosci. 6:237-245 (1990)). In some embodiments, parameters for a particular embodiment in which identity can be narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever can be shorter. According to this embodiment, if the subject sequence can be shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that can be lateral to the N- and C-terminal of the subject sequence, which can be not matched/aligned with a corresponding subject residue, as a percent of the total bases of the query sequence. A determination of whether a residue can be matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which can be not matched/aligned with the query sequence, can be considered for the purposes of manually adjusting the percent identity score. That is, only query residue positions outside the farthest N- and C-terminal residues of the subject sequence can be considered for this manual correction. For example, a 90-residue subject sequence can be aligned with a 100-residue query sequence to determine percent identity. The deletion occurs at the N-terminus of the subject sequence, and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% can be subtracted from the percent identity score calculated by the FASTDB program. If the remaining 90 residues were perfectly matched, the final percent identity can be 90%. In another example, a 90-residue subject sequence can be compared with a 100-residue query sequence. This time the deletions can be internal deletions, so there can be no residues at the N- or C-termini of the subject sequence which can be not matched/aligned with the query. In this case, the percent identity calculated by FASTDB can be not manually corrected. Once again, only residue positions outside the N- and C-terminal ends of the subject sequence, as displayed in the FASTDB alignment, which can be not matched/aligned with the query sequence can be manually corrected for.

The term “substantially” or “essentially” may refer to a qualitative condition that exhibits an entire or nearly total range or degree of a feature or characteristic of interest. In some cases, substantially encapsulated may refer to near complete encapsulation of a substance or compound. For example, substantially encapsulated may comprise a particle that is at least about: 70%, 75%, 80%, 85%, 90%, 95%, 99%, or 100% encapsulated. In some cases, substantially may refer to at least about: 70%, 75%, 80%, 85%, 90%, 95%, 99%, or 100% of the total range or degree of a feature or characteristic of interest.

The term “at least partially” may refer to a qualitative condition that exhibits a partial range or degree of a feature or characteristic of interest. In some cases, at least partially encapsulated may refer to a partial encapsulation of a substance or compound. For example, at least partially encapsulated may comprise a particle that is at least about: 5%, 10%, 20%, 30%, 40%, 50%, 60%, 70%, 75%, 80%, 85%, 90%, 95%, 99%, or 100% encapsulated.

The term “in vivo” may be used to describe an event that takes place in a subject's body.

The term “ex vivo” may be used to describe an event that takes place outside of a subject's body. An “ex vivo” assay may not be performed on a subject. Rather, it may be performed upon a sample separate from a subject. An example of an “ex vivo” assay performed on a sample may be an “in vitro” assay.

The term “in vitro” may be used to describe an event that takes place contained in a container for holding laboratory reagent such that it may be separated from the living biological source organism from which the material may be obtained. In vitro assays may encompass cell-based assays in which cells alive or dead are employed. In vitro assays may also encompass a cell-free assay in which no intact cells are employed.

As used herein, the term ‘about’ a number may refer to that number plus or minus 10% of that number. The term ‘about’ a range may refer to that range minus 10% of its lowest value and plus 10% of its greatest value.

As used herein, a “derivative” of a compound disclosed herein, can refer to a chemical substance related structurally a compound disclosed herein. A derivative can be made from the structurally-related parent compound in one or more steps. The general physical and chemical properties of a derivative can be similar to a parent compound. A derivative can be, for example, an analog or a homolog.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit may be achieved with the eradication or amelioration of one or more of the physiological symptoms associated with the underlying disorder such that an improvement may be observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the terms “administration,” and “administering” may refer to the manner in which an active agent is presented to a subject. Administration can be accomplished by various routes such as oral, parenteral, transdermal, inhalation, implantation, etc.

As used herein, the term “pulmonary administration” may represent any method of administration in which an active agent can be administered through the pulmonary route by inhaling an aerosolized liquid or powder form (nasally or orally). Such aerosolized liquid or powder forms are traditionally intended to substantially release and or deliver the active agent to the mucosal membrane and epithelium of the lungs. In the context of this disclosure, the active agent is in powder form.

As used herein, the term “nominal load” or “total load” may refer to the total amount of formulation packaged or partitioned for administration to a subject. For example, the nominal load is the total amount of powder formulation that is enclosed in a capsule for use with an inhaler.

As used herein, the term “nominal dose” or “total dose” may refer to the total amount or mass of active agent packaged or partitioned for administration to a subject. For example, the nominal dose is the total amount of active agent that is enclosed in a capsule for use with an inhaler.

As used herein, the term “emitted dose” (ED (%)) may refer to the mass of an active agent that is emitted from a dry powder inhaler aerosolization device as a percentage of a nominal dose mass. Powder that exhibits high flow rate often results in higher ED (%).

As used herein, the term “fine particle fraction” or “fine particle fraction from the emitted dose” (% FPF(ED)) may refer to the mass of active agent having an aerodynamic diameter below about 10 or 5 μm as a percentage of an emitted dose mass. The FPF is often used to evaluate the efficiency of aerosol deaggregation.

As used herein, the term “respirable fraction” (RF (%)) may refer to the mass of an active agent that is below a certain aerodynamic cutoff size as a percentage of a nominal dose mass. Also known as the fine particle fraction from the total dose (FPF(TD)). Fine particle fraction may also be calculated as a percentage of the emitted dose (FPF(ED)). The respirable fraction represents the proportion of powder aerosol that can enter the deep respiratory tract. In some cases the RF cutoff size can be an aerodynamic diameter of less than about 10 μm, less than about 7 μm, or less than or about 5 μm. The respirable fraction may be determined using an inertial sampling device.

As used herein, the term “mass median aerodynamic diameter” (MMAD) refers to the mass median aerodynamic diameter of airborne particles at which 50% of particles by mass are larger and 50% are smaller. In other words, it is the median of the aerodynamic particle size distribution as a function of particle mass. The percentages of mass less than the stated aerodynamic diameters versus the aerodynamic diameters are plotted logarithmically. The MMAD is taken as the intersection of the line with the 50% cumulative percent. Computational methods can also be applied.

As used herein, the term “unit dose” or “dosage form” may be used interchangeably and may be meant to refer to pharmaceutical drug products in the form in which they are marketed for use, with a specific mixture of active ingredients and inactive components or excipients, in a particular configuration, and apportioned into a particular dose to be delivered. The term “unit dose” may also sometimes encompass non-reusable packaging, although the FDA distinguishes between unit dose “packaging” or “dispensing”. More than one unit dose may refer to distinct pharmaceutical drug products packaged together, or to a single pharmaceutical drug product containing multiple drugs and/or doses. The term “unit dose” may also sometimes refer to the particles comprising a pharmaceutical composition, and to any mixtures involved. Types of unit doses may vary with the route of administration for drug delivery, and the substance(s) being delivered. A solid unit dose may be the solid form of a dose of a chemical compound used as a pharmaceutically acceptable drug or medication intended for administration or consumption.

As used herein, the term “fine particle fraction” or “fine particle fraction from the emitted dose” may refer to the mass of active agent having an aerodynamic diameter below about: 5 μm, 6 μm, 7 μm, 8 μm, 9 μm, or 10 μm. In some instances, the cutoff size may be less than or equal to an aerodynamic diameter of about 5 μm. In some instances, the cutoff size may be less than or equal to an aerodynamic diameter of about 6.4 μm. In some instances, the cutoff size may be less than or equal to an aerodynamic diameter of about 7 μm or about 8 μm. In some instances, the fine particle fraction may be often used to evaluate the efficiency of aerosol deaggregation. In some cases, fine particle fraction may be the mass of active agent having an aerodynamic diameter below about: 5 μm, 6 μm, 7 μm, 8 μm, 9 μm, or 10 jm as a percentage of an emitted dose mass. In some cases, fine particle fraction may be the mass of active agent having an aerodynamic diameter of more than about: 5 μm, 6 μm, 7 μm, 8 μm, 9 μm, or 10 μm as a percentage of an emitted dose mass. In some cases, fine particle fraction may be the mass of active agent having an aerodynamic diameter from about: 1 μm to about 10 μm, 1 μm to about 5 μm, 3 μm to about 6 μm, 4 μm to about 7 μm, 6 μm to about 12 μm or about 7 μm to about 10 μm as a percentage of an emitted dose mass. For example, a composition described herein may have a fine particle fraction of more than, less than, or equal to about: 20%, 25%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 75%, 80%, 85%, 90% or 95% upon aerosolization.

The terms “therapeutic agent,” “active agent,” “active pharmaceutical ingredient,” “API,” “pharmaceutically active agent,” and “pharmaceutical,” and “drug” may be used interchangeably herein to refer to a substance having a pharmaceutical, pharmacological, psychosomatic, or therapeutic effect. Further, when these terms are used, or when a particular active agent is specifically identified by name or category, it is understood that such recitation is intended to include the active agent per se, as well as pharmaceutically acceptable, pharmacologically active derivatives thereof, or compounds significantly related thereto, including without limitation, salts, pharmaceutically acceptable salts, N-oxides, prodrugs, active metabolites, isomers, fragments, solvates (such as hydrates), polymorphs, pseudopolymorphs, and esters.

As used herein, “pharmaceutically acceptable salt” may refer to pharmaceutical drug molecules, which may be formed as a weak acid or base, chemically made into their salt forms, most frequently as the hydrochloride, sodium, or sulfate salts. Drug products synthesized as salts may enhance drug dissolution, boost absorption into the bloodstream, facilitate therapeutic effects, and increase its effectiveness. Pharmaceutically acceptable salts may also facilitate the development of controlled-release dosage forms, improve drug stability, extend shelf life, enhance targeted drug delivery, and improve drug effectiveness.

As used herein, “laser diffraction” may refer to a method for particle size analysis, which consists of scattering laser light off an assembly of particles and collecting the scattered light using a spatial array of detectors. The signal from the detectors may be a pattern of scattered/diffracted light vs. angle. This pattern may result from many particles being illuminated by the laser light source at the same time, where all of their individual scattered/diffracted light rays mix together at each detector element.

As used herein, “particle size analyzer” may refer to an instrument for particle size analysis, particle size measurement, or simply particle sizing.

As used herein, “particle size analysis” may refer to the collective name of the technical procedures, or laboratory techniques which determines the size range, and/or the average (mean), median or mode size of the particles, or droplets in a powder or liquid sample.

As used herein, “time to peak plasma concentration” may refer to the time required for a drug to reach peak concentration in plasma. Peak concentration in plasma may be usually defined as the plasma concentration that a drug achieves in a specified compartment or test area of the body after the drug has been administered and before the administration of a second dose.

As used herein, “HPLC” may refer to high-performance liquid chromatography (formerly referred to as high-pressure liquid chromatography), which is a technique in analytical chemistry used to separate, identify, and quantify each component in a mixture. HPLC may be a common technique used in pharmaceutical development, as it may be a method to ensure product purity.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine.

The section headings used herein are for organizational purposes only and are not to be construed as limiting the subject matter described.

### Pharmaceutical Formulations

Disclosed herein are devices, systems and methods for producing, packaging, and delivering stable powdery pharmaceutical compositions. The active ingredient of these stable powdery formulations may comprise steroid receptor agonists. The active ingredients may further comprise additional ingredients as disclosed below. In certain instances, the dry powdery compositions may include inactive ingredients such as excipients, carriers and/or diluents.

In certain instances, the dry powdery pharmaceutical compositions can be inhalable. For example, the dry powdery pharmaceutical compositions can be administered in a dry powdered inhaler In other instances, the dry powdery pharmaceutical compositions can be for oral administration. For example, the dry powdery pharmaceutical compositions can be administered in a capsule-in capsule formulation. In another example, the dry powdery pharmaceutical compositions can be administered as microencapsulated particles with multiple layers.

### Active Ingredients and Pharmaceutically Acceptable Salts

An active pharmaceutical ingredient may be any substance or mixture of substances intended to be used in the manufacture of a drug (medicinal) product and that, when used in the production of a drug, becomes an active ingredient of the drug product. Such substances may be intended to furnish pharmacological activity or other direct effect in the diagnosis, cure, mitigation, treatment, or prevention of disease or to affect the structure or function of the body. In some aspects, an active pharmaceutical ingredient or salt thereof may be formulated as a powder. For example, inhalable hormone receptor agonists formulations disclosed herein may be formulated as a powder using the methods described herein. In some cases, an active ingredient comprises a pharmaceutical compound. In some cases, a pharmaceutical compound comprises an active ingredient.

In certain aspects, active ingredient or ingredients may be present in different crystal forms. The different crystalline forms of the same compound can have an impact on one or more physical properties, such as stability, solubility, melting point, bulk density, flow properties, bioavailability, etc.

### Steroid Receptor Agonists

In some aspects, the active pharmaceutical ingredients may comprise a steroid receptor agonists or pharmaceutically acceptable salts thereof. In some cases, a steroid receptor agonist is referred to herein as a steroid. In some aspects, the steroid receptor agonist can be for an androgen receptor such as NR3C4. In some aspects, the steroid receptor agonist can be for an estrogen receptor such as Era, Erp, GPR30, ER-X, Gq-mER or a combination thereof. In some aspects the steroid receptor agonist can be for a progesterone receptor such as NR3C3.

In certain cases, the steroid receptor agonist is a steroid such as: pregnenolone, progesterone, deoxycorticosterone, corticosterone, aldosterone, 17α-hydroxypregnenolone, 17α-progesterone, 11-deoxycortisol, cortisol, dehydroepiandrosterone, androstenedione (A4), androstenediol (A5), testosterone, dihydrotestosterone (DHT), dehydroepiandrosterone sulfate (DHEA-S), estrone, estradiol, salts of any of these, and derivatives of any of these. In some cases, the steroid receptor agonist is testosterone or a salt thereof. In some cases, a testosterone can comprise testosterone enanthate, testosterone cypionate, testosterone propionate, testosterone undecanoate, a methyltestosterone or any combination thereof.

In certain cases, the steroid is an androgen. An androgen may be considered any natural or synthetic steroid hormone that regulates the development and maintenance of male characteristics in vertebrates by binding to androgen receptors. This may include the embryological development of male sex organs, and the development of male secondary sex characteristics at puberty. Androgens may be synthesized in the testes, the ovaries, and the adrenal glands. Adrenal androgens are composed of 19-carbon steroids synthesized in the zona reticularis, the innermost layer of the adrenal cortex. Adrenal androgens function as weak steroids (though some are precursors), and the subset includes dehydroepiandrosterone (DHEA), dehydroepiandrosterone sulfate (DHEA-S), androstenedione (A4), and androstenediol (A5) a combination of these, or a salt or derivative of these.

Androstenedione (A4) is an androgenic steroid produced by the testes, adrenal cortex, and ovaries. While androstenedione is converted metabolically to testosterone and other androgens, it is also the parent structure of estrone. Androstenediol (A5) is a steroid metabolite of DHEA and the precursor to sex hormones testosterone and estradiol. Androsterone is a chemical byproduct created during the breakdown of androgens, or derived from progesterone, that also exerts minor masculinizing effects, but with one-seventh the intensity of testosterone. It is found in approximately equal amounts in the plasma and urine of both males and females. Dihydrotestosterone (DHT) is a metabolite of testosterone, and a more potent androgen than testosterone in that it binds more strongly to androgen receptors. It is produced in the skin and reproductive tissue.

In certain cases, the steroid is an estrogen. Estrogens may be considered a category of sex hormones responsible for development and regulation of the female reproductive system and secondary sex characteristics. Among these are estrone (E1), estradiol (E2), estriol (E3), estetrol (E4), which can be known asestranes. Estrogens of the disclosure may include a combination of estranes, salts thereof, and/or derivatives of estranes. The estrogen steroid hormones estradiol, estrone, and estriol are estra-1,3,5(10)-trienes. All of the different forms of estrogen are synthesized from androgens, specifically testosterone and androstenedione, by the enzyme aromatase. Minor endogenous estrogens, the biosyntheses of which do not involve aromatase, include 27-hydroxycholesterol, dehydroepiandrosterone (DHEA), 7-oxo-DHEA, 7α-hydroxy-DHEA, 16α-hydroxy-DHEA, 70-hydroxyepiandrosterone, androstenedione (A4), androstenediol (A5), 3α-androstanediol, and 30-androstanediol. Those of the present disclosure may include a combination of these, salt thereof, or derivatives of these. Minor endogenous estrogens, the biosyntheses of which do not involve aromatase, include 27-hydroxycholesterol, dehydroepiandrosterone (DHEA), 7-oxo-DHEA, 7α-hydroxy-DHEA, 16α-hydroxy-DHEA, 70-hydroxyepiandrosterone, androstenedione (A4), androstenediol (A5), 3α-androstanediol, and 30-androstanediol. This disclosure also may include a combination of these, salts thereof, and/2029775 or derivatives of these minor endogenous estrogens. In some cases, composition herein can comprise estrone, estradiol, estriol, estetrol, a salt of any of these, a derivative of any of these, or any combination thereof.

In certain instances, the steroid is a progestin. Progestin steroid hormones may include progesterone (P4), 16α-hydroxyprogesterone (16α-OHP), 17α-hydroxyprogesterone (17α-OHP), 20α-dihydroprogesterone (20α-DHP), 20β-dihydroprogesterone (20β-DHP), 5α-dihydroprogesterone (5α-DHP), 5β-dihydroprogesterone (5β-DHP), 3β-dihydroprogesterone (3β-DHP), 11-deoxycorticosterone (DOC), 5α-dihydrodeoxycorticosterone (5α-DHDOC), a combination of these, a salt of these, or a derivative of these. In some cases, a composition herein can comprise a progestin. In some cases, a progestin can comprise a pregnane, an estrange, a gonane, a salt of any of these, or any combination thereof. In some cases, a progestin can comprise a norethindrone, a norethindrone acetate, an ethynodiol, an ethynodiol diacetate, a levonorgestrel, a norgestrel, a desogestrel, a norgestimate, a drospirenone, a norethynodrel, or any combination thereof. In some cases, a progestin can comprise medroxyprogesterone acetate, nomegestrol acetate, a gestodene, or any combination thereof. In some cases, a composition herein can comprise progesterone, a salt thereof, or a derivative thereof.

In certain cases, a hormone may not be a steroid hormone, but may be instead a peptide hormone that is an agonist for a peptide hormone receptor.

### GLP-1 Receptor Agonists

In certain instances, a peptide hormone may be a glucagon-like peptide 1 receptor agonist (GLP-1), GLP-1-RA, gastric inhibitory peptide receptor agonist including incretitins exenatide, lixisenatide, incretin, albiglutide, liraglutide, dulaglutide, glargine, glimepiride, semaglutide, sitagliptin, tirzepatide, taspoglutide, efpeglenatide, a pharmaceutically acceptable salt of these, a derivative of these, or a combination of these. The agonist may bind to the GLP-1 receptor. The interaction may be used for the treatment of diabetes such as type 1 diabetes, type 2 diabetes, type 3c diabetes (pancreatogenic diabetes), a prediabetes, a gestational diabetes, an obesity, or a combination thereof. In certain instances, treatment of subjects with one of these agonists may be used for subjects having coronary artery disease (CAD), also called coronary heart disease (CHD), ischemic heart disease (IHD), myocardial ischemia, or a combination thereof. In some cases, a GLP-1 agonist can be used to treat a dementia. In some cases, a dementia can comprise an Alzheimer's disease, a vascular dementia, a Lewy body disease, a frontotemporal dementia, an alcohol related dementia, a young onset dementia, a mild cognitive impairment, or any combination thereof.

In some cases, a composition herein can comprise a polypeptide from Table 1, for example any one of SEQ ID NOs 1-12, a salt of any of these, or any combination thereof. In certain instances, a composition herein can comprise a polypeptide that may be greater than, or equal to: 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs 1-12, or a salt of any of these. An incretin known as gastric inhibit polypeptide may comprise the polypeptide sequence SEQ ID NO 1. In certain instances, the sequence may be about: 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO 1 listed in Table 1.

Liraglutide may comprise the polypeptide sequence SEQ ID NO 2. In certain instances, the liraglutide may be 100 percent identical to SEQ ID NO 2. In certain instances, the sequence may be about: 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO 2 listed in Table 1.

Exenatide may comprise the polypeptide sequence SEQ ID NO 3. In certain instances, the exenatide may be 100 percent identical to SEQ ID NO 3. In certain instances, the sequence may be about: 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO 3 listed in Table 1.

Lixisenatide may comprise the polypeptide sequence SEQ ID NO 4. In certain instances, the lixisenatide may be 100 percent identical to SEQ ID NO 4. In certain instances, the sequence may be at least about: 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO 4 listed in Table 1.

Albiglutide may comprise the polypeptide sequence SEQ ID NO 5. In certain instances, the albiglutide may be 100 percent identical to SEQ ID NO 5. In certain instances, the sequence may be at least about: 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO 5 listed in Table 1.

Dulaglutide is a recombinant DNA produced polypeptide analog of GLP-1 that is covalently linked to each Fc arm of human immunoglobulin. One chain of dulaglutide may comprise the polypeptide sequence SEQ ID NO 6. In certain instances, the sequence may be at least about: 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO 6 listed in Table 1.

Taspoglutide may comprise the polypeptide sequence SEQ ID NO 7. In certain instances, the taspoglutide may be 100 percent identical to SEQ ID NO 7. In certain instances, the sequence may be at least about: 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO 7 listed in Table 1.

A GLP-1 Receptor agonist may comprise the polypeptide sequence SEQ ID NO 12. In certain instances, the GLP-1 Receptor antagonist may be 100 percent identical to SEQ ID NO 12. In certain instances, the sequence may be at least about: 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO 12 listed in Table 1.

### Growth Hormone Receptor Agonists

In certain cases, a human growth hormone receptor agonist comprising a human growth hormone peptide, a salt, a derivative, or a combination of these may be used alone or in a pharmaceutical formulation to treat a condition. In some cases, a condition may comprise a deficiency of human growth hormone. In certain cases, the human growth hormone receptor agonist may be somatotrophin, somatropin, human chorionic somatomammotropin, a derivative of these, a salt of these, or a combination of these. In certain other cases, a human growth hormone is derived from a stimulator of human growth hormone. These may include, but are not limited to: somatocrinin, ghrelin, clonidine, α4β2 nicotinic agonists, glucagon, niacin, salts of these, derivatives of these, or a combination of these. In certain cases a human growth hormone receptor agonist or a promotor of human growth hormone receptor agonists may be used to increase calcium retention, promote muscle mass increase, promote lipolysis, promote internal organ growth, reduce liver uptake of glucose, promote gluconeogenesis in the liver, promote maintenance and function of pancreatic islets, stimulate the immune system, improve cognitive function, treat turner syndrome, treat chronic kidney failure, treat Prader-Willi syndrome, treat intrauterine growth restriction, treat severe idiopathic short stature, maintain muscle mass from wasting in subjects suffering from AIDS, promote anti-aging, promote lean body mass, promote the maintenance or increase in body density, treating multiple sclerosis, promote weight loss, treat fibromyalgia, treat heart failure, treat Chron's disease, treat ulcerative colitis, and treat burns.

Human growth hormone may comprise the polypeptide sequence SEQ ID NO 8. In certain instances, the sequence may be 100 percent identical to SEQ ID NO 8. In certain instances, the sequence may be at least about: 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO 8 listed in Table 1.

Somatotrophin may comprise the peptide sequence SEQ ID NO 9. In certain instances, the sequence of somatotrophin may be 100 percent identical to SEQ ID NO 9. In certain instances, the sequence may be at least about: 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO 9 listed in Table 1.

Somatropin may comprise the peptide sequence SEQ ID NO 10. In certain instances, the sequence of somatropin may be 100 percent identical to SEQ ID NO 10. In certain instances, the sequence may be at least about: 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO 10 listed in Table 1.

Human chorionic somatomammotropin may comprise the peptide sequence SEQ ID NO 11. In certain instances, the sequence of human chorionic somatomammotropin may be 100 percent identical to SEQ ID NO 11. In certain instances, the sequence may be at least about: 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO 11 listed in Table 1.

TABLE 1
Sequences of Therapeutic Polypeptides

| Sequence | | |
|---|---|---|
| ID | Description | Amino Acid Sequence |
| SEQ. ID. | Incretin; NCBI | MVATKTFALLLLSLFLAVGLGEKKEGHFSALPSLPVGSH |
| NO. 1 | Accession No. | AKVSSPQPRGPRYAEGTFISDYSIAMDKIHQQDFVNWLL |
| | NP_004114 | AQKGKKNDWKHNITQREARALELASQANRKEEEAVEP |
| | | QSSPAKNPSDEDLLRDLLIQELLACLLDQTNLCRLRSR |
| SEQ. ID. | Liraglutide | HAEGTFTSDVSSYLDEGQAAK*EPIAWLVRGRG with * |
| NO. 2 | | being a palmitoyl-E-OH |
| SEQ. ID. | Exenatide | HGEGTFTSDLSKQMEEEAVRLFIEWLKNGGPSSGAPPPS |
| NO. 3 | | |
| SEQ. ID. | Lixisenatide | HGEGTFTSDLSKQMEEEAVRLFIEWLKNGGPSSGAPPSK |
| NO. 4 | | KKKKK |
| SEQ. ID. | Albiglutide | HGEGTFTSDV SSYLEGQAAK EFIAWLVKGR |
| NO. 5 | | HGEGTFTSDV SSYLEGQAAK EFIAWLVKGR |
| | | DAHKSEVAHR FKDLGEENFK ALVLIAFAQY |
| | | LQQCPFEDHV KLVNEVTEFA KTCVADESAE |
| | | NCDKSLHTLF GDKLCTVATL RETYGEMADC |
| | | CAKQEPERNE CFLQHKDDNP NLPRLVRPEV |
| | | DVMCTAFHDN EETFLKKYLY EIARRHPYFY |
| | | APELLFFAKR YKAAFTECCQ AADKAACLLP |
| | | KLDELRDEGK ASSAKQRLKC ASLQKFGERA |
| | | FKAWAVARLS QRFPKAEFAE VSKLVTDLTK |
| | | VHTECCHGDL LECADDRADL AKYICENQDS |
| | | ISSKLKECCE KPLLEKSHCI AEVENDEMPA |
| | | DLPSLAADFV ESKDVCKNYA EAKDVFLGMF |
| | | LYEYARRHPD YSVVLLLRLA KTYETTLEKC |
| | | CAAADPHECY AKVFDEFKPL VEEPQNLIKQ |
| | | NCELFEQLGE YKFQNALLVR YTKKVPQVST |
| | | PTLVEVSRNL GKVGSKCCKH PEAKRMPCAE |
| | | DYLSVVLNQL CVLHEKTPVS DRVTKCCTES |
| | | LVNRRPCFSA LEVDETYVPK EFNAETFTFH |
| | | ADICTLSEKE RQIKKQTALV ELVKHKPKAT |
| | | KEQLKAVMDD FAAFVEKCCK ADDKETCFAE |
| | | EGKKLVAASQ AALGL |
| SEQ. ID. | Dulaglutide | HGEGTFTSDVSSYLEEQAAKEFIAWLVKGGGGGGGSGG |
| NO. 6 | | GGSGGGGSAESKYGPPCPPCPAPEAAGGPSVFLFPPKPK |
| | | DTLMISRTPEVTCVVVDVSQEDPEVQFNWYVDGVEVHN |
| | | AKTKPREEQFNSTYRVVSVLTVLHQDWLNGKEYKCKVS |
| | | NKGLPSSIEKTISKAKGQPREPQVYTLPPSQEEMTKNQVS |
| | | LTCLVKGFYPSDIAVEWESNGQPENNYKTTPPVLDSDGS |
| | | FFLYSRLTVDKSRWQEGNVFSCSVMHEALHNHYTQKSL |
| | | SLSLG |
| SEQ. ID. | Taspoglutide | HXEGTFTSDVSSYLEGQAAKEFIAWLVKXR, where X is |
| NO. 7 | | α-aminoisobutyric acid |
| SEQ. ID. | Human growth | MFPTIPLSRLFDNAMLRAHRLHQLAFDTYQEFEEAYIPK |
| NO. 8 | hormone; | EQKYSFLQNPQTSLCFSESIPTPSNREETQQKSNLELLRIS |
| | NCBI | LLLIQSWLEPVQFLRSVFANSLVYGASDSNVYDLLKDLE |
| | accession No. | EGIQTLMGRLEDGSPRTGQIFKQTYSKFDTNSHNDDALL |
| | K02382.1 | KNYGLLYCFRKDMDKVETFLRIVQCRSVEGSCGF |
| SEQ. ID. | Somatotrophin; | MATGSRTSLLLAFGLLCLPWLQEGSAFPTIPLSRLFDNA |
| NO. 9 | NCBI | MLRAHRLHQLAFDTYQEFEEAYIPKEQKYSFLQNPQTSL |
| | Accession No. | CFSESIPTPSNREETQQKSNLELLRISLLLIQSWLEPVQFLR |
| | P01241 | SVFANSLVYGASDSNVYDLLKDLEEGIQTLMGRLEDGSP |
| | | RTGQIFKQTYSKFDTNSHNDDALLKNYGLLYCFRKDMD |
| | | KVETFLRIVQCRSVEGSCGF |
| SEQ. ID. | Somatropin; | FPTIPLSRLFDNAMLRAHRLHQLAFDTYQEFEEAYIPKEQ |
| NO. 10 | KEEG drug | KYSFLQNPQTSLCFSESIPTPSNREETQQKSNLELLRISLL |
| | database entry | LIQSWLEPVQFLRSVFANSLVYGASDSNVYDLLKDLEEG |
| | D02691 | IQTLMGRLEDGSPRTGQIFKQTYSKFDTNSHNDDALLKN |
| | | YGLLYCFRKDMDKVETFLRIVQCRSVEGSCGF |
| SEQ. ID. | Human | MAPGSRTSLLLAFALLCLPWLQEAGAVQTVPLSRLFDH |
| NO. 11 | chorionic | AMLQAHRAHQLAIDTYQEFEETYIPKDQKYSFLHDSQTS |
| | somatomammo | FCFSDSIPTPSNMEETQQKSNLELLRISLLLIESWLEPVRF |
| | tropin; NCBI | LRSMFANNLVYDTSDSDDYHLLKDLEEGIQTLMGRLED |
| | Accession No. | GSRRTGQILKQTYSKFDTNSHNHDALLKNYGLLYCFRK |
| | AAA52115 | DMDKVETFLRMVQCRSVEGSCGF |
| SEQ. ID. | GLP-1 | HGEGX¹FX¹SDVSSYLEGQAAKEFX²AWLVKGR, wherein |
| NO. 12 | Receptor | X¹ is D-allo-threonine, X² is D-alloisolucene, and A |
| | agonist | is D-alanine |

In certain cases, steroid hormone receptor agonists or their pharmaceutically acceptable salts thereof are used to treat depression, obesity, low libido, underdeveloped muscle mass, underdeveloped secondary sex characteristics, depression, underdeveloped body hear growth, underdeveloped voice deepening, underdeveloped tendons and ligaments, and lack of spermatogenesis. In certain instances these agonists or their pharmaceutically acceptable salts thereof can be used to treat conditions characterized by low estrogen levels such as regulation of the female reproductive system, lack of female secondary sex characteristics, low vaginal lubrication, underdeveloped uterine growth, unwanted bone reabsorption, low bone formation, undesirably low levels of body fat, thinning vaginal walls, infertility, regulation of ovulation, regulation of menstruation, endometriosis, depression, obsessive compulsive disorder, eating disorders, and low libido which can be found in both men and women and the like. In certain instances, these agonists or their pharmaceutically acceptable salts thereof can be used to treat conditions characterized by low progesterone levels characterized by low progesterone levels such as uterine cancer, cervical cancer, inability to prevent pregnancy, inability to maintain a pregnancy, unwanted preterm labor, unwanted lactation during pregnancy, insufficient breast development for breastfeeding, skin aging, libido regulation, and the like.

In certain cases, pharmaceutical compositions provided herein may pertain to a pharmaceutical composition comprising steroid receptor agonist or a pharmaceutical salt thereof that increases muscle mass.

In some cases, the steroid hormone agonist or salt thereof or a metformin or a salt thereof can be administered orally or by a capsule or by a capsule in capsule. In some cases, a capsule may release the pharmaceutical composition for intranasal or inhalation administration by the use of an inhaler. In some cases, the steroid hormone agonist or salt thereof or a metformin or a salt thereof can be administered by a food or beverage in a multi-layered microencapsulated particle. In some cases, the steroid hormone agonist or salt thereof or a metformin or a salt thereof can be contained in a gummy and can be administered as a gummy.

In some aspects, the composition may further comprise: another set of active pharmaceutical ingredients or salts thereof. For example, a second, third, or fourth different set of active pharmaceutical ingredients. In some aspects, the additional pharmaceutical ingredients or salts thereof may be administered in parallel or consecutively to enhance the efficacy of the first set of active pharmaceutical ingredients or salts.

### Co-Therapeutics

In some aspects, a composition may further comprise: an additional set of active pharmaceutical ingredients or salts thereof which may be administered in parallel or consecutively to enhance the efficacy of a hormone receptor agonist. In some aspects, a second different set of active pharmaceutical ingredients or salts may be administered in parallel or consecutively to enhance the efficacy of a steroid receptor agonist. In some aspects, a composition may comprise two or more different sets of active pharmaceutical ingredients or salt thereof which may be administered in parallel or consecutively to enhance the hormone receptor agonist.

In some cases, a first therapeutic is a steroid receptor agonist or salt thereof and a second therapeutic is a cannabinoid. In some cases, the steroid receptor agonist is an estrogen, progesterone, or a combination thereof is an estrogen steroid and the second is the cannabinoid.

In some cases, a first therapeutic is GLP-1 receptor agonist or salt thereof and the second therapeutic is a cannabinoid. In some cases, a first therapeutic is a growth hormone receptor agonist or a salt thereof and the second therapeutic is a cannabinoid.

In some embodiments, a first set of active pharmaceutical ingredients or salts thereof may be administered in parallel or consecutively with a second different set of active pharmaceutical ingredients. In some aspects, a second different set of active pharmaceutical ingredients or salts thereof may not be comprised in the powdery pharmaceutical composition. In some aspects, a second different set of active pharmaceutical ingredients or salts thereof not comprised in the powdery pharmaceutical composition may be administered concurrently, in parallel, or consecutively to the first set of active pharmaceutical ingredients.

### Cannabinoids

The term “cannabinoid” can refer to a chemical compound that shows direct or indirect activity at a cannabinoid receptor. In some cases, a composition herein can comprise a cannabinoid. In some cases, a cannabinoid can be comprised in a powdery pharmaceutical composition. In some cases, a cannabinoid can be at least partially encapsulated. In some instances, a cannabinoid can comprise a phytocannabinoid. In some instances, a cannabinoid can comprise a endocannabinoid. In some instances, an endocannabinoid can comprise anandamide (arachidonoyl ethanolamide) or 2-arachidonoyl glycerol (2-AG). In some cases, a cannabinoid can be a full spectrum cannabinoid. In some cases, a cannabinoid can be a broad-spectrum cannabinoid. Examples of cannabinoids include, but are not limited to, tetrahydrocannabinol (THC), cannabidiol (CBD), cannabinol (CBN), cannabigerol (CBG), cannabichromene (CBC), cannabicyclol (CBL), cannabivarin (CBV), cannabidivarin (CBDV), cannabichromevarin (CBCV), cannabigerovarin (CBGV), cannabigerol monomethyl ether (CBGM), and cannabidiolic acid (CBDA). In some cases, a cannabinoid can comprise cannabielsoin (CBE), cannabicitran (CBT), 10-Oxo-delta-6a-tetrahydrocannabinol (OTHC), cannabichromanon, cannabifuran, cannabiglendol, cannabiripsol, or cannbicitran. In some cases, a cannabinoid can comprise a cannabinoid from Table 2.

TABLE 2

| Cannabinoid | Abbreviation |
|---|---|
| Cannabigerovarin | CBGV |
| Cannabigerovarinic acid | CBGVA |
| Cannabigerol | CBG |
| Cannabigerolic acid | CBGA |
| O-Methlycannabigerol | |
| Cannabigerolic acid methylether | |
| Cannabinerolic acid | |
| Cannabinerol | |
| Carmagerol | |
| rac-6′-Epoxycannabigerol | |
| rac-6′-Epoxycannabigerolic acid | |
| rac-6′-Epoxycannabinerol | |
| rac-6′-Epoxycannabinerolic acid | |
| γ-Eudesmyl cannabigerolate | |
| γ-Cadinyl cannabigerolate | |
| Sesquicannabigerol | |
| Deprenyl O-methyl cannabigerolic acid | |
| (Amorfrutin 2) | |
| 5-Acetyl-4-hydroxycannabigerol | |
| Acetylcannabigeroquinol | |
| Cannabigeroquinone | |
| Abnormal cannabigerol | |
| Acetyl abnormal hydrocannabigeroquinol | |
| Abnormal cannabigeroquinol | |
| 2′-Hydroxy-1′,2′-dihydrocannabichromene | |
| (Cyclo-CBG) | |
| Cannabiorcichromene | |
| Cannabiorcichromenic acid | |
| Chlorcannabiorcichromenic acid | |
| Cannabivarichromene | |
| Cannabichromevarinic acid | |
| Cannabichromene | CBC |
| Cannabichromenic acid | |
| 4-Acetoxycannabichromene | |
| Anthopogochromenic acid | |
| Confluentin | |
| Daurichromenic acid | |
| 8′-Hydroxyisocannabichromene | |
| 4-Acetoxycannabichromene | |
| Cannabidiorcol | |
| Cannabidivarin | CBDV |
| Cannabidivarinic acid | |
| nor-Cannabidiol | |
| Cannabidiol | CBD |
| O-Methlycannabidiol | |
| O-Propylcannabidiol | |
| O-Pentylcannabidiol | |
| Cannabidiolic acid | CBDA |
| CBDA-THC ester | |
| Ferruginene C | |
| Cannabioxepane | |
| Cannabinodivarin | |
| Cannabinodiol | |
| Cannabifuran | |
| Dehydrocannabifuran | |
| trans-Δ-8-Tetrahydrocannabinol | |
| trans-Δ-8-Tetrahydrocannabinolic acid | |
| 10α-Hydroxy trans-Δ-8- | |
| tetrahydrocannabinol | |
| 10β-Hydroxy trans-Δ-8- | |
| tetrahydrocannabinol | |
| 11-Acetoxy-Δ-8-tetrahydrocannabinoic acid | |
| 10-Hydroxy-9-oxo-Δ-8- | |
| tetrahydrocannabinol | |
| Δ-9-trans-Tetrahydrocannabiorcol | |
| Δ-9-trans-Tetrahydrocannabiorcolic acid | |
| Δ-9-trans-Tetrahydrocannabivarin | |
| Δ-9-trans-Tetrahydrocannabivarinic acid | |
| Δ-9-trans-nor-Tetrahydrocannabinol | |
| Δ-9-trans-nor-Tetrahydrocannabinolic acid | |
| Δ-9-trans-Tetrahydrocannabinol | |
| Δ-9-trans-Tetrahydrocannabinolic acid A | |
| Δ-9-trans-Tetrahydrocannabinolic acid B | |
| 8α-Hydroxy-Δ-9-trans-tetrahydrocannabinol | |
| 8β-Hydroxy-Δ-9-trans-tetrahydrocannabinol | |
| 8-Oxo-Δ-9-trans-tetrahydrocannabinol | |
| O-Propyl-Δ-9-trans-tetrahydrocannabinol | |
| O-Pentyl-Δ-9-trans-tetrahydrocannabinol | |
| 2-Formyl-Δ-9-trans-tetrahydrocannabinol | |
| β-Fenchyl Δ-9-trans- | |
| Tetrahydrocannabinolate | |
| α-Fenchyl Δ-9-trans- | |
| Tetrahydrocannabinolate | |
| Bornyl Δ-9-trans-Tetrahydrocannabinolate | |
| epi-Bornyl Δ-9-trans- | |
| Tetrahydrocannabinolate | |
| α-Terpinyl Δ-9-trans- | |
| Tetrahydrocannabinolate | |
| 4-Terpinyl Δ-9-trans- | |
| Tetrahydrocannabinolate | |
| γ-Eudesmyl Δ-9-trans- | |
| Tetrahydrocannabinolate | |
| α-Cadinyl Δ-9-trans- | |
| Tetrahydrocannabinolate | |
| Hexahydrocannabinol | |
| Hydroxy Δ-9,11-hexahydrocannabinol | |
| Methylen-bis Δ-9-trans- | |
| Tetrahydrocannabinol (Cannabisol) | |
| Tetrahydrocannabinol epoxide | |
| Δ-9-trans-Tetrahydrocannabinol glycol | |
| (cannabiripsol) | |
| 6a,7,10a-Trihydroxy-Δ-9- | |
| tetrahydrocannabinol | |
| Δ-9-cis-Tetrahydrocannabivarin | |
| Δ-9-cis-Tetrahydrocannabinol | |
| Cannabicitran (citrilidene-cannabis) | CBT |
| Cannabiorcicitran | |
| Bis-nor cannabitriol | |
| Bis-nor-Cannabitriol isomer | |
| 10-O-Ethyl bis-nor cannabitriol | |
| Isocannabitriol | |
| Cannabitriol | |
| Cannabitriol isomer | |
| 10-O-Ethyl cannabitriol isomer | |
| 10-Oxo-Δ6a(10a)-tetrahydrocannabinol | |
| 9,10-Anhydrocannabitirol | |
| Cannabiglendol | |
| 7,8-Dehydro-10-O-ethylcannabitriol | |
| Δ-7-cis-Isotetrahydrocannabivarin | |
| Δ-7-trans-Isotetrahydrocannabivarin | |
| Δ-7-trans-isotetrahydrocannabinol | |
| Bis-nor-cannabielsoin | |
| Bis-nor-Cannabielsoic acid B | |
| Cannabielsoin | CBE |
| Cannabielsoic acid A | |
| Cannabielsoic acid B | |
| Ferruginene A | |
| Ferruginene B | |
| Cannabiorcicyclol | |
| Cannabiorcicyclolic acid | |
| Cannabicyclovarin | |
| Cannabicyclol | CBL |
| Cannabicyclolic acid | |
| Anthopogocyclolic acid | |
| Rhododaurichromanic acid A | |
| Cannabiorcol | |
| nor-Cannabivarin | |
| Cannabivarin | CBV |
| nor-Cannabinol | |
| Cannabinol | CBN |
| Cannabinolic acid | |
| O-Methylcannabinol | |
| O-Propylcannabinol | |
| O-Penthylcannabinol | |
| 7-Hydroxcannabinol | |
| 8-Hydroxycannabinol | |
| 8-Hydroxcannabinolic acid | |
| 7,8-Dihydrocannabinol | |
| 4-Terpenyl cannabinolate | |
| Cannabicoumaronone | |
| Cannabicourmarononic acid | |
| Bisnor-Cannabichromanone | |
| Cannabichromanone | |
| (6aR)-Cannabichromanone B | |
| (6aR)-Cannabichromanone C | |
| Cannabichromanone | |
| Cannabimovone | |
| Anhydrocannabimovone | |
| Demethyldecarboxyamorfrutin A | |
| Tetrahydrocannabiphorol | THCP |
| Cannabidiphorol | CBDP |
| Cannabimovone | CBM |

Tetrahydrocannabinol, commonly referred to as “THC”, Isolate THC, or Full Spectrum THC, can include Tetrahydrocannabinol Delta-7, Tetrahydrocannabinol Delta-8, Tetrahydrocannabinol Delta-9, Tetrahydrocannabinol Delta-11, Tetrahydrocannabinol Delta-10, Tetrahydrocannabinol Delta-13, Tetrahydrocannabivarin (THCV) and Tetrahydrocannabinolic acid (THCA). In some instances, THC can comprise trans-THC, cis-THC or both. In some cases, THC can exist as a stereoisomer, such as, (+)-trans-THC; (−)-trans-THC; (+)-cis-THC and (−)-cis-THC. In some cases, cis-TCH can comprise, (+)-cis-THC, (−)-cis-THC, or both. In some cases, trans-THC can comprise (+)-trans-THC, (−)-trans-THC, or both. In some cases, a composition can comprise a ratio (weight to weight) of trans-THC to cis-THC of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, a composition can comprise a ratio (weight to weight) of cis-THC to trans-THC of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, CBD can comprise trans-CBD. In some cases, trans-CBD can comprise (+)-trans-CBD, (−)-trans-CBD, or both. In some instances, CBD can comprise an enantiomer, or a diastereomer. In some instances, CBD can comprise a racemate. In some instances, CBD can comprise trans-CBD, cis-CBD or both. In some cases, CBD can comprise (1R,6R)-CBD, (1R,6S)-CBD, (1S,6R)-CBD, (1S,6S)-CBD, or a combination thereof. In some cases, a composition can comprise aratio (weight to weight) of trans-CBD to cis-CBD of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, a composition can comprise a ratio (weight to weight) of cis-CBD to trans-CBD of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some instances, CBD can be a powder, a liquid, an oil, an emulsion, an aerosol, a solid or a combination thereof. In some cases, CBD can be at least partially water soluble. In some instances, a cannabinoid can be a racemate. In some instances, a cannabinoid can comprise an isomer. In some instances, a cannabinoid can comprise an enantiomer, or a diastereomer. In some cases, a composition can comprise a ratio (weight to weight) of THC to CBD of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, a composition can comprise a ratio (weight to weight) of CBD to THC of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, CBD can be mixed in a composition with Δ8-THC, Δ9-THC, Δ10-THC or a combination thereof. In some cases, a composition can comprise a ratio (weight to weight) of Δ8-THC, Δ9-THC, or Δ10-THC to CBD of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, a composition can comprise a ratio (weight to weight) of CBD to Δ8-THC, Δ9-THC, or Δ10-THC of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some instances, a cannabinoid or a salt thereof can be derived from hemp. In some instances, a cannabinoid or a salt thereof can be derived from cannabis. In some instances, a tetrahydrocannabinol or a salt thereof can be derived from hemp. In some instances, tetrahydrocannabinol Delta-8 or a salt thereof can be derived from hemp. In some instances, tetrahydrocannabinol Delta-8 or a salt thereof can be derived from cannabis. In some cases, a cannabinoid can be a synthetic cannabinoid or a salt thereof. In some cases, a cannabinoid can include a derivative of a cannabinoid or a salt thereof. In some instances, a cannabinoid can comprise an isomer of a cannabinoid.

In some embodiments, active pharmaceutical ingredients or salts thereof can comprise a THC, a CBD, a pharmaceutically acceptable salt of any of these, or any combination thereof. In some embodiments, active pharmaceutical ingredients can be THC or a pharmaceutically acceptable salt thereof.

### Nonsteroidal Anti-Inflammatory Drugs (NSAIDs)

In some embodiments, a further active ingredient can comprise a NSAID or a salt thereof, and the NSAID can comprise aspirin, ibuprofen, naproxen, diflunisal, dexibuprofen, oxaprozin, fenoprofen, indomethacin, tolmetin, celecoxib, clonixin, ketoprofen, a pharmaceutically acceptable salt of any of these, or any combination thereof. In some cases, a composition herein can comprise a NSAID. In some cases, a NSAID can be comprised in a powdery pharmaceutical composition. In some cases, a NSAID can be at least partially encapsulated.

### Phosphodiesterase Inhibitors

In some embodiments, the active pharmaceutical ingredients can comprise phosphodiesterase inhibitors or pharmaceutically acceptable salts thereof. In some cases, a phosphodiesterase inhibitor can be comprised in a powdery pharmaceutical composition. In some cases, a phosphodiesterase inhibitor can be at least partially encapsulated. In some cases, a further active ingredient can comprise a phosphodiesterase inhibitor. In some embodiments, the phosphodiesterase inhibitors can be phosphodiesterase type 5 inhibitors (PDE5 inhibitors). In some embodiments, the phosphodiesterase type 5 inhibitors can include Sildenafil Citrate (Viagra), Tadalafil (Cialis) Avanafil (Stendra), and Vardenafil Hydrochloride (Levitra). In some cases, a PDE-V inhibitor can comprise sildenafil, tadalafil, avanafil, vardenafil, an ester thereof, a salt thereof, or any combination thereof. In some cases, a PDE-V inhibitor can comprise mirodenafil, udenafil, lodenafil, zaprinast, icariin, an ester of any of these, a pharmaceutically acceptable salt of any of these, or any combination thereof. In some cases, a PDE-V inhibitor can comprise lodenafil carbonate. In some cases, a phosphodiesterase inhibitor can comprise a selective phosphodiesterase inhibitor, a nonselective phosphodiesterase inhibitor, a PDE-I selective inhibitor, a PDE-II selective inhibitor (e.g. EHNA (erythro-9-(2-hydroxy-3-nonyl)adenine)), a PDE-III selective inhibitor, a PDE-IV selective inhibitor, a PDE-V selective inhibitor, a PDE-VI selective inhibitor, a PDE-VII selective inhibitor, a PDE-IX selective inhibitor, a PDE-X selective inhibitor, a PDE-XI selective inhibitor, a pharmaceutically acceptable salt of any of these, or any combination thereof. In some cases, an active pharmaceutical ingredient can comprise oxindole, inamrinone, anagrelide, cilostazol, mesembrenone, rolipram, ibudilast, roflumilast, apremilast, cisaborole, sildenafil, tadalafil, vardenafil, udenafil, avanafil, dipyridamole, quinazoline, paraxanthine, papaverine, a pharmaceutically acceptable salt of any of these, an ester of any of these, or any combination thereof. In some cases, a PDE5 inhibitor or a salt thereof such as sildenafil or a salt thereof can be administered in a composition comprising a cannabinoid described herein.

In some cases, active pharmaceutical ingredients or salts may comprise a promoter of nitric oxide synthesis such as arginine, ascorbic acid, folic acid, tetrahydrobiopterin, a pharmaceutically acceptable salt of any of these, a derivative of these, or a combination of these.

### Other Therapeutics

In some cases, an active pharmaceutical ingredient or a salt thereof may comprise an anti-diabetic medication or a salt thereof. In some cases, an anti-diabetic medication can be comprised in a powdery pharmaceutical composition. In some cases, an anti-diabetic medication can be at least partially encapsulated. In some cases, the anti-diabetic medication is a sulfonylurea such as tolbutamide, acetohexamide, glimepiride, glipizide, glyburide, glyclopyramide, gliquidone, tolazamide, chlorpropamide or any combination thereof. In some cases, the anti-diabetic medication is a glinide such as repaglinide, nateglinide or a combination thereof. In some cases, the anti-diabetic drug is a biguanuide such as metformin, phenformin, buformin, or a combination thereof. In some cases, the anti-diabetic medication is a thiazolidinedione such as rosiglitazone, pioglitazone, troglitazone, lobeglitazone, ciglitazone, darglitazone, englitazone, netoglitazone, balaglitazone or any combination thereof. In some cases, the anti-diabetic drug is an alpha glucosidase inhibitor such as miglitol, acarbose, voglibose or any combination thereof. In some cases, the anti-diabetic medication is a dipeptidyl peptidase-4 inhibitor such as vildagliptin, sitagliptin, saxagliptin, linagliptin, alogliptin, septagliptin, teneligliptin, gemigliptin or any combination thereof. In certain cases, the anti-diabetic medication is a glycosuric such as dapagliflozin, canagliflozin, empagliflozin, remogliflozin or any combination thereof.

In some cases, a composition herein can comprise a metformin. In some embodiments, an active pharmaceutical ingredient or a salt thereof may comprise a metformin or a salt thereof. In some cases, the metformin can be in the form of a dry powder manufactured by the methods described herein (e.g., spray drying). In some cases, metformin particles can be at least partially encapsulated. For example, the metformin particles can be at least partially encapsulated by one, two, three, or more layers of coating material. In some cases, a coating material can comprise a trehalose, a hydroxypropyl methylcellulose (HPMC), a fumaryl diketopiperazine (FDKP), a 1,2-distearoyl-sn-glycero-3-phosphocholine, a hydroxypropyl methylcellulose acetate succinate (HPMCAS), a povidone, a copovidone, a lactose, a phospholipid or any combination thereof. In some cases, the coating material can comprise an enteric coating. In some cases, an enteric coating can comprise methyl methacrylate (MMA) or a salt thereof. In some cases, an enteric coating can comprise a plant fiber, a polymer, a shellac, a wax, a fatty acid, a plastic, or a combination thereof. In some cases, an enteric coating can comprise a methyl acrylate-methacrylic acid copolymer, a cellulose acetate phthalate (CAP), a cellulose acetate succinate, hydroxypropyl methyl cellulose phthalate, a HPMCAS, a polyvinyl acetate phthalate (PVAP), a methyl methacrylate-methacrylic acid copolymer, a shellac, a cellulose acetate trimellitate, a sodium alginate, a zein, an enteric coating solution (an ethylcellulose, a medium chain triglycerides, an oleic acid, a sodium alginate, a stearic acid), or a combination thereof. In some cases, an excipient, a carrier, and/or a diluent can be added to a composition comprising the at least partially encapsulated metformin particles.

In some cases, the at least partially encapsulated metformin particles can be added to a gummy. In some cases, the at least partially encapsulated metformin particles can have a particle diameter ranging from about 1 to 200 micrometers, or a plurality of metformin particles have a mean or median particle diameter of about 1 micrometers to about 200 micrometers, as measured by a particle analyzer using laser diffraction. In some cases, the at least partially encapsulated metformin particles can have a particle diameter of less than, more than, or equal to about: 1 μm, 5 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, or 200 μm. In some cases, the at least partially encapsulated metformin particles can be formulated to be any particle size disclosed herein. In some cases, the gummy comprising the at least partially encapsulated metformin particles can comprise about 10 mg to about 5000 mg of metformin. In some cases, a gummy comprising the at least partially encapsulated metformin particles can comprise more than, less than, or equal to about: 10 mg, 50 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, or 3000 mg of metformin. In some cases, a gummy comprising the at least partially encapsulated metformin particles can comprise about: 100 mg to about 3000 mg; 500 mg to about 2500 mg; 100 mg to about 1000 mg, 500 mg to about 1500 mg, or about 100 mg to about 500 mg of metformin. In some cases, the gummy comprising the at least partially encapsulated metformin can be in a kit. In some cases, the kit can comprise a container.

In some cases, the at least partially encapsulated metformin particles can be administered in the form of a beverage or a food (e.g., a gummy). In some embodiments, a gummy can provide at least substantially controlled release of the metformin or the salt thereof over time. In some cases, the gummy provides substantially instant release of the metformin or the salt thereof for example, in the stomach or in the small intestine. In some cases, the at least partially encapsulated metformin particles can be used to treat a disease or condition. In some cases, the at least partially encapsulated metformin particles can be administered to a subject who has a disease or condition. In some cases, the disease or condition can comprise a type 1 diabetes, a type 2 diabetes, a type 3c diabetes (pancreatogenic diabetes), a prediabetes, a metabolic syndrome, a gestational diabetes, a polycystic ovary syndrome, obesity, weight gain from an antipsychotic medicine, a liver disease, a cardiovascular disease, a renal disease, or any combination thereof. In some cases, the at least partially encapsulated metformin particles can be administered to a subject for life extension, for health span extension, or both. In some aspects, active pharmaceutical ingredients or salts may comprise an antibiotic, an antiviral, an antiparasitic, a diuretic, a beta-blocker, an ACE inhibitor, an angiotensin II receptor blocker, a calcium channel blocker, an alpha blocker, a cancer chemotherapeutic, an anti-inflammatory steroid, an immunomodulator, a pharmaceutically acceptable salt of any of these, or any combination thereof. In some cases, an antibiotic may comprise a penicillin, a cephalosporin, a tetracycline, an aminoglycoside, a macrolide, clindamycin, a sulfonamide, a trimethoprim, a metronidazole, a quinolone, or a nitrofurantoin. An antiviral may comprise an acyclovir, peramivir, zanamivir, oseltamivir phosphate, remdesivir, balozavir marboxil, a salt of any of these or any combination thereof. In some cases, an active pharmaceutical ingredient or salt thereof may comprise a potassium channel blocker such as dalfampridine or a salt thereof. In some cases, an active pharmaceutical ingredient or salt thereof may comprise levodopa, carbidopa, or a salt thereof. In some cases, the pharmaceutical ingredients may comprise beta blockers (0-blockers), calcium blockers, angiotensin converting enzyme inhibitors, angiotensin receptor blockers, Nebivolol, CYP3A4 inhibitors, ketoconazole (Nizoral), itraconazole (Sporanox), erythromycin, saquinavir, clarithromycin, HIV protease inhibitors, alpha-adrenergic blocking agents (α-blockers), salts thereof, or any combination thereof.

In some cases, the pharmaceutical ingredients may comprise nitrates, nitric oxide, nitric oxide generating components, nitrite salts, nitrate salts, sodium nitrates, potassium nitrates, vitamin C, ascorbic acid, L-arginine, L-citrulline, vitamin B12, magnesium ascorbate, sodium ascorbate, potassium ascorbate, antihypertensive agents, diuretics, salts thereof, or any combination thereof.

In some aspects, the pharmaceutical composition has metabolites that may be pharmacologically active, retaining, at least partially, the potency of the parent drug or the parent pharmaceutical component.

### Salts

In some aspects, the pharmaceutical composition comprises the salt of the pharmaceutically active ingredient, wherein the salt comprises an organic salt, an inorganic salt, or any combination thereof. In some cases, an organic salt may comprise a phosphinate (e.g., sodium hypophosphite), a hydrazinium salt, a urate, a diazonium salt, an oxalate salt, a tartrate, a choline chloride. An example of an inorganic salt may be sodium chloride, calcium chloride, magnesium chloride, sodium bicarbonate, potassium chloride, sodium sulfate, calcium carbonate, calcium phosphate, or any combination thereof.

In certain aspects, the salt comprises an HCl salt, an ascorbic acid salt, a mandelic acid salt, an aspartic acid salt, a carbonic acid salt, a citric acid salt, a formic acid salt, a glutamic acid salt, a lactic acid salt, a lauric acid salt, a maleic acid salt, a borate salt, a bitartrate salt, a palmitic acid salt, a phosphoric acid salt, or any combination thereof. In certain aspects, the pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like.

### Excipients and Other Non-Active Ingredients

In some aspects, a pharmaceutical composition can comprise a pharmaceutically acceptable: excipient, diluent, and/or carrier. As used herein, an excipient may refer to a substance formulated alongside the active ingredient of a medication, included for the purpose of long-term stabilization, bulking up solid formulations that contain potent active ingredients in small amounts, and/or to confer a therapeutic enhancement on the active ingredient(s) in the final dosage form. In some cases, a diluent can comprise water, or a saline. In some cases, a carrier can comprise a water, a sugar solution, a honey, or a saline. Excipients may facilitate drug absorption, reduce viscosity, or enhance solubility. Excipients may also facilitate the handling of the active ingredients, improve in vitro stability, and/or extend pharmaceutical product shelf life. Excipient selection may vary with the route of administration for drug delivery, the unit dose, as well as the active ingredients comprising the composition.

In some aspects, an excipient may comprise anhydrous calcium phosphate, dihydrate calcium phosphate, hydroxypropyl methylcellulose, croscarmellose sodium, GMO-free croscarmellose sodium, carbomers, magnesium aluminometasilicate, mannitol, povidone (PVP), crospovidone, sorbitol, dimethicone, sodium stearyl fumarate, sodium starch glycollate, hydroxypropylcellulose, native corn starch, modified corn starch, carrageenan, alginates, silicon dioxide, microcrystalline cellulose, carboxymethylcellulose sodium, alginates, carboxymethylcellulose (CMC), sodium carboxymethylcellulose (Na CMC), carbomers, natural gums, sorbitol, maltitol, glucose syrup, silicones, carbomers, fatty alcohols, alcohols, carbohydrates, petrolatum derivatives, butters, waxes, DMSO, esters, fatty acids, oil-in-water (O/W) emulsifiers, water-in-oil (W/O) emulsifiers, silicas, fumed silicas, polysorbates, isopropyl myristate, cellulosic derivates, xanthan gum, propylenglycol, noveon AA-1 polycarbophyl, dimethyl isosorbate, polysilicone elastomer 1100, polysilicone elastomer 1148P, preservatives, flavors, colors, functional coatings, aesthetic coatings, a pharmaceutically acceptable salt of any of these, or any combination thereof.

In some cases, a pharmaceutically acceptable excipient may comprise acacia, acesulfame potassium, acetic acid, glacial, acetone, acetyl tributyl citrate, acetyl triethyl citrate, agar, albumin, alcohol, alginic acid, aliphatic polyesters, alitame, almond oil, alpha tocopherol, aluminum hydroxide adjuvant, aluminum oxide, aluminum phosphate adjuvant, aluminum stearate, ammonia solution, ammonium alginate, ascorbic acid, ascorbyl palmitate, aspartame, attapulgite, bentonite, benzalkonium chloride, benzethonium chloride, benzoic acid, benzyl alcohol, benzyl benzoate, boric acid, bronopol, butylated hydroxyanisole, butylated hydroxytoluene, butylparaben, calcium alginate, calcium carbonate, calcium phosphate, dibasic anhydrous, calcium phosphate, dibasic dihydrate, calcium phosphate, tribasic, calcium stearate, calcium sulfate, canola oil, carbomer, carbon dioxide, carboxymethylcellulose calcium, carboxymethylcellulose sodium, carrageenan, castor oil, castor oil, hydrogenated, cellulose (e.g., microcrystalline, powdered, silicified microcrystalline, acetate, acetate phthalate) ceratonia, cetostearyl alcohol, cetrimide, cetyl alcohol, cetylpyridinium chloride, chitosan, chlorhexidine, chlorobutanol, chlorocresol, chlorodifluoroethane, chlorofluorocarbons, chloroxylenol, cholesterol, citric acid monohydrate, colloidal silicon dioxide, coloring agents, copovidone, corn oil, cottonseed oil, cresol, croscarmellose sodium, crospovidone, cyclodextrins, cyclomethicone, denatonium benzoate, dextrates, dextrin, dextrose, dibutyl phthalate, dibutyl sebacate, diethanolamine, diethyl phthalate, difluoroethane, dimethicone, dimethyl ether, dimethyl phthalate, dimethyl sulfoxide, dimethylacetamide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol palmitostearate, ethylene vinyl acetate, ethylparaben, fructose, fumaric acid, gelatin, glucose, glycerin, glyceryl behenate, glyceryl monooleate, glyceryl monostearate, glyceryl palmitostearate, glycofurol, guar gum, hectorite, heptafluoropropane, hexetidine, hydrocarbons, hydrochloric acid, hydroxyethyl cellulose, hydroxyethylmethyl cellulose, hydroxypropyl cellulose, hydroxypropyl cellulose, low-substituted, hydroxypropyl starch, hypromellose, hypromellose acetate succinate, hypromellose phthalate, honey, imidurea, inulin, iron oxides, isomalt, isopropyl alcohol, isopropyl myristate, isopropyl palmitate, kaolin, lactic acid, lactitol, lactose, anhydrous, lactose, monohydrate, lactose, spray-dried, lanolin, lanolin alcohols, lanolin, hydrous, lauric acid, lecithin, leucine, linoleic acid, macrogol hydroxystearate, magnesium aluminum silicate, magnesium carbonate, magnesium oxide, magnesium silicate, magnesium stearate, magnesium trisilicate, malic acid, maltitol, maltitol solution, maltodextrin, maltol, maltose, mannitol, medium-chain triglycerides, meglumine, menthol, methylcellulose, methylparaben, mineral oil, mineral oil and lanolin alcohols, monoethanolamine, monosodium glutamate, monothioglycerol, myristic acid, neohesperidin dihydrochalcone, nitrogen, nitrous oxide, octyldodecanol, oleic acid, oleyl alcohol, olive oil, palmitic acid, paraffin, peanut oil, pectin, petrolatum, petrolatum and lanolin alcohols, phenol, phenoxyethanol, phenylethyl alcohol, phenylmercuric acetate, phenylmercuric borate, phenylmercuric nitrate, phosphoric acid, polacrilin potassium, poloxamer, polycarbophil, polydextrose, polyethylene glycol, polyethylene oxide, polymethacrylates, poly(methyl vinyl ether/maleic anhydride), polyoxyethylene alkyl ethers, polyoxyethylene castor oil derivatives, polyoxyethylene sorbitan fatty acid esters, polyoxyethylene stearates, polyvinyl acetate phthalate, polyvinyl alcohol, potassium alginate, potassium benzoate, potassium bicarbonate, potassium chloride, potassium citrate, potassium hydroxide, potassium metabisulfite, potassium sorbate, povidone, propionic acid, propyl gallate, propylene carbonate, propylene glycol, propylene glycol alginate, propylparaben, 2-pyrrolidone, raffinose, saccharin, saccharin sodium, saponite, sesame oil, shellac, simethicone, sodium acetate, sodium alginate, sodium ascorbate, sodium benzoate, sodium bicarbonate, sodium borate, sodium chloride, sodium citrate dihydrate, sodium cyclamate, sodium hyaluronate, sodium hydroxide, sodium lactate, sodium lauryl sulfate, sodium metabisulfite, sodium phosphate, dibasic, sodium phosphate, monobasic, sodium propionate, sodium starch glycolate, sodium stearyl fumarate, sodium sulfite, sorbic acid, sorbitan esters (sorbitan fatty acid esters), sorbitol, soybean oil, starch, starch (e.g., pregelatinized, sterilizable maize), stearic acid, stearyl alcohol, sucralose, sucrose, sugar, compressible, sugar, confectioner's, sugar spheres, sulfobutylether b-cyclodextrin, sulfuric acid, sunflower oil, suppository bases, hard fat, talc, tartaric acid, tetrafluoroethane, thaumatin, thimerosal, thymol, titanium dioxide, tragacanth, trehalose, triacetin, tributyl citrate, triethanolamine, triethyl citrate, vanillin, vegetable oil, hydrogenated, water, wax, anionic emulsifying, wax (e.g., camauba, cetyl esters, microcrystalline, nonionic emulsifying, white, yellow), xanthan gum, xylitol, zein, zinc acetate, zinc stearate, or any combination thereof.

In some aspects, a pharmaceutically acceptable excipient may comprise a carbohydrate, an alginate, a povidone, a carbomer, a flavor, a natural gum, a silicone, an alcohol, a butter, a wax, a fatty acid, a preservative, a pharmaceutically acceptable salt of any of these, or any combination thereof. In some aspects, a pharmaceutically acceptable excipient may comprise a carbohydrate. In some instances, the carbohydrate may comprise a lactose, a microcrystalline a cellulose, a cellulose, a mannitol, a sorbitol, a starch, a starch glycolate, a hydroxypropyl a methylcellulose, a hydroxypropyl methylcellulose acetate succinate, a cyclodextrin, a maltodextrin, a trehalose, a croscarmellose sodium, a corn starch, a carrageenan, a sorbitol, a maltitol, a glucose, a pharmaceutically acceptable salt of any of these, or any combination thereof. In some aspects, a pharmaceutically acceptable excipient may comprise a lactose. In some instances, lactose may comprise a milled lactose, a sieved lactose, a micronized lactose, a spray dried lactose, at least substantially anhydrous lactose, a monohydrate lactose, or a combination thereof. In some cases, an excipient can comprise fumaryl diketopiperazine (FDKP). In some cases, an excipient can comprise 1,2-distearoyl-sn-glycero-3-phosphocholine (DSPC).

In some aspects, a dosage such as an inhalable dosage is in a lipid formulation. In certain instances, the liposome may be a positively charged liposome. In certain instances, the liposome may be a negatively charged liposome. In certain cases, a lipid may be a saturated phospholipid such as dipalmitoyl phosphotidylcholine (DPPC). In certain instances, a lipid can be a glycerophospholipid. Glycerophospholipids can be phosphatidylcholine (PC), phosphatidylethanolamine (PE), phosphatidylserine (PS), phosphatidic acid (PA), phosphatidylinositol (PI), phosphatidylglycerol (PG) cardiolipin (CL), or any combination thereof. In certain cases, lipids may be phospholipids such as soybean phosphatidylcholine (SPC), hydrogenated soybean phosphatidylcholine (HSPC), egg sphingomyelin (ESM), egg phosphatidylcholine (EPC), dimyristoyl phosphatidylcholine (DMPC), dipalmitoyl phosphatidylcholine (DPPC), dioleoyl phosphatidylcholine (DOPC), distearoyl phosphatidylcholine (DSPC), dimyristoyl phosphatidylglycerol (DMPG), dipalmitoyl phosphatidylglycerol (DPPG), dioleoyl phosphatidylglycerol (DOPG), distearoyl phosphatidylglycerol (DSPG), dimyristoyl phosphatidylethanolamine (DMPE), dipalmitoyl phosphatidylethanolamine (DPPE), dioleoyl phosphatidylethanolamine (DOPE), dimyristoyl phosphatidylserine (DMPS), dipalmitoyl phosphatidylserine (DPPS) or any combination thereof.

In some aspects, the active ingredient or pharmaceutically acceptable salt thereof may be contained at least in part within an excipient. In some aspects, the active ingredient or pharmaceutically acceptable salt thereof may be contained at least in part in an excipient. In some aspects, the active ingredient may be contained within a pore of an excipient. The “pore” of the excipient may refer to excipient particles that have been engineered to have open or closed pore structures. Porous excipient particles may be carriers of pharmaceutically active ingredients. Porous excipient particles may have a large surface area, stable structure, adjustable pore sizes, tunable dissolution, diffusion, or distribution, and well-defined surface properties. Porous excipient particles may facilitate sustained-release unit doses.

In some aspects, in addition to the active pharmaceutical ingredients or salts thereof, the compositions may further comprise inactive ingredients selected from the group consisting of microcrystalline cellulose, anhydrous dibasic calcium phosphate, croscarmellose sodium, magnesium stearate, hypromellose, titanium dioxide, lactose, triacetin, mannitol, xylitol, sorbitol, sugar alcohols, cellulose, cellulose esters, cellulose ethers, modified celluloses, starch, modified starches, polysaccharides, oligosaccharides, disaccharides, saccharides, gelatin, polyvinylpyrrolidone, polyethylene glycol, binders, flavorants, colorants, FD & C Blue #2 aluminum lake, magnesium stearate, anti-adherent agents, stearate salts, sweeteners, silica, lubricants, or any combination thereof.

### Inhalable Formulations

Advancements in engineering and process automation allow for enhanced methods of manufacturing to achieve consistent active ingredient particle size in the 1.0-10.0 micrometer size, which contributes to proper absorption in the lungs. A drug may be processed using spray drying technology to control the particle size and particle size distribution. Spray drying may produce active ingredient particle size in the 1.0-10.0-micrometer range. This particle size may be needed when a drug is administered by inhalation or by an intranasal route of administration for absorption into the lung alveolar. In some instances, the particle may be microencapsulated to enhance bioavailability. This route of administration may result in a rapid introduction of the drug into the blood stream and may require lower dosing when compared to oral intake of a capsule or tablet. In some instances, introducing encapsulated hormone receptor agonists into the lungs via inhalation, may allow hormone receptor agonists to reach the blood stream within 5 minutes.

In some aspects the compositions may comprise one or more of: an active ingredient or salts thereof, excipients, and inactive ingredients. In some cases, a pharmaceutical composition may comprise particles. In some cases, particles may comprise an excipient (e.g., a pharmaceutically acceptable excipient), an active ingredient, an encapsulated active ingredient or any combination thereof. In some cases, the compositions may comprise a pharmaceutical composition. In some instances, a composition may comprise particles of a pharmaceutically acceptable excipient. In some instances, a composition may comprise particles of an active ingredient.

As used herein, “coating material” may refer to a material added via a pharmaceutical coating process by which an essentially dry, outer layer of coating material may be applied to the surface of a dosage form. Coating dosage forms may be used to improve stability (light protection, moisture and gas barrier), facilitate administration, or modify the drug release behavior from the dosage form. The coating materials may be used to enable the immediate release of the drug, delay the release of the drug (such as in enteric coatings), or sustain the release of the drug from the dosage form over extended periods of time. Coating materials may include film coating formulations, which usually contain a polymer, a plasticizer, a colorant, opacifier, a solvent, and a vehicle. In some cases, a coating material may refer to the coating material used in the coating of a particle of an active ingredient to create an encapsulated particle.

In some aspects, a composition may comprise a mixture of particles described herein. In some aspects, the particles may be mixed in a substantially homogenous mixture. In some instances, at least a portion of the particles of the pharmaceutically acceptable excipient may have a particle diameter ranging from about 50 micrometers to about 200 micrometers, as measured by a particle size analyzer using laser diffraction. In some cases, at least a portion of the active ingredient particles may have a particle diameter ranging from about 500 nanometers to about 15 micrometers, or about 1 micrometer to about 20 micrometers, as measured by a particle size analyzer using laser diffraction. In some instances, in a human clinical trial, the powdery pharmaceutical composition, when inhaled into the lungs, may provide in at least part of the humans in the clinical trial a time to peak plasma concentration (Tmax) of the active ingredient or the salt thereof ranging from about 1 minute to about one hour, or from about 1 minute to about ten minutes. In some aspects, the Tmax of the active ingredient or the salt thereof ranging from about 1 min to about 5 min, about 1 min to about 10 min, about 1 min to about 20 min, about 1 min to about 25 min, about 1 min to about 30 min, about 1 min to about 40 min, about 1 min to about 50 min, about 1 min to about 60 min, about 5 min to about 10 min, about 5 min to about 20 min, about 5 min to about 25 min, about 5 min to about 30 min, about 5 min to about 40 min, about 5 min to about 50 min, about 5 min to about 60 min, about 10 min to about 20 min, about 10 min to about 25 min, about 10 min to about 30 min, about 10 min to about 40 min, about 10 min to about 50 min, about 10 min to about 60 min, about 20 min to about 25 min, about 20 min to about 30 min, about 20 min to about 40 min, about 20 min to about 50 min, about 20 min to about 60 min, about 25 min to about 30 min, about 25 min to about 40 min, about 25 min to about 50 min, about 25 min to about 60 min, about 30 min to about 40 min, about 30 min to about 50 min, about 30 min to about 60 min, about 40 min to about 50 min, about 40 min to about 60 min, or about 50 min to about 60 min.

In some aspects, when inhaled into the lungs in a human clinical trial, the powdery pharmaceutical composition operates mechanistically such that in at least a portion of the humans in the clinical trial, a majority of the particles of the pharmaceutically acceptable excipient deposit onto the oropharynx.

In some aspects, the weigh to weight ratio of: a) the particles of the pharmaceutically acceptable excipient and b) the active ingredient particles ranges from about 1:1 to about 10000:1. In some aspects, the weight to weight ratio of: a) the particles of the pharmaceutically acceptable excipient and b) the active ingredient particles ranges from about 1:1 to about 20:1, about 1:1 to about 15:1, about 1:1 to about 10:1, about 1:1 to about 5:1, about 1:1 to about 2:1, about 2:1 to about 20:1, about 2:1 to about 15:1, about 2:1 to about 10:1, about 2:1 to about 5:1, about 5:1 to about 20:1, about 5:1 to about 15:1, about 5:1 to about 10:1, about 10:1 to about 15:1, about 10:1 to about 20:1, about 15:1 to about 20:1, about 18:1 to about 25:1, or about 25:1 to about 30:1. In some aspects, the weight to weight ratio of a) the particles of the pharmaceutically acceptable excipient and b) the active ingredient particles may be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or 30:1 In some aspects, the weight to weight ratio of a) the particles of the pharmaceutically acceptable excipient and b) the active ingredient particles ranges from about 1:1 to about 1:10, about 1:1 to about 1:8, about 1:1 to about 1:5, about 1:1 to about 1:2, about 1:2 to about 1:10, about 1:2 to about 1:8, about 1:2 to about 1:5, about 1:5 to about 1:10, about 1:5 to about 1:8, about 1:8 to about 1:10.

In some aspects, at least a portion of the particles of the pharmaceutical excipient and the active ingredient particles may not be covalently bound to each other.

In some aspects, a solid active ingredient is spherical or substantially spherical and is not encapsulated. In other cases, the solid active ingredient is spherical or substantially spherical and is encapsulated. In certain cases, the spherical or substantially spherical active ingredient is mixed with an excipient. In certain cases, the excipient can also be spherical or substantially spherical. In certain cases, the excipient may comprise lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, corn starch, carrageenan, sorbitol, maltitol, glucose, fumaryl diketopiperazine (FDKP) a pharmaceutically acceptable salt of any of these, or any combination thereof.

### Orally Administered Formulations

Disclosed herein are devices, systems and methods for producing, packaging, and delivering stable powdery compositions via oral administration. In some cases, a microencapsulated particle can contain multiple coatings and be administered via inhalation. In some cases, a microencapsulated particle can contain multiple coatings and be orally administered. For example, a microencapsulated particle with one or more coatings (e.g., shells) can be added to a liquid and administered orally. In some embodiments, a microencapsulated particle can be configured (for example, with one or more layers of an enteric coating) to at least partially release an active ingredient in: the mouth, the esophagus, the small intestine, the duodenum, the jejunum, the ileum, the cecum, the colon, the ascending colon, the traverse colon, the descending colon, the sigmoid colon, the rectum, the anus, or any combination thereof. In some cases, microencapsulated particles can be added to a capsule, such as a capsule in capsule composition and be orally administered.

In some embodiments, the compositions can be spray dried. In some embodiments, the spray dried powder can be processed through a fluid bed to apply a polymer barrier or enteric coating. In some embodiments, the compositions can comprise one or more coatings. For example, a microencapsulated particle can have 1, 2, 3, 4, 5, or more than 5 coatings comprising the same or different coating material. Overall, the spray dried particles can individually have a particle diameter ranging from about 20 micrometers to about 200 micrometers or have a mean or median particle diameter of about 20 micrometers to about 200 micrometers, as measured by a particle analyzer using laser diffraction.

A first coating may comprise trehalose. In certain cases, the first coating may comprise trehalose and a hydroxypropyl methylcellulose acetate succinate (HPMCAS). In other instances, the first coating material may comprise a cyclodextrin, a maltodextrin, a povidone, a copovidone or any combination thereof. In certain instances, the first coating may further comprise a cannabinoid or a pharmaceutically acceptable salt thereof. In some cases, a first coating may comprise a hydroxypropyl methylcellulose (HPMC), a hydroxypropyl methylcellulose acetate succinate (HPMCAS), a cyclodextrin, a maltodextrin, a povidone, a copovidone, or any combination thereof. In some cases, a first coating can comprise any wall or shell material.

In some cases, the first coating can be substantially encapsulated by one or more enteric coatings. An enteric coating comprises a barrier, such as a polymer barrier, that can be applied to a composition (for example a microencapsulated particle) to prevent dissolution or disintegration in the stomach. In some cases, this can enable the active ingredient to bypass the stomach to the small intestines before the active ingredient is released. In some cases, a wall material such as an additional coating on a previously microencapsulated particle can comprise an enteric coating. In some cases, the first coating of a microencapsulated particle can comprise an enteric coating. In some cases, an enteric coating can comprise methyl methacrylate (MMA). In some cases, an enteric coating can comprise a plant fiber, a shellac, a wax, a fatty acid, a plastic, or a combination thereof. In some cases, an enteric coating can comprise a methyl acrylate-methacrylic acid copolymer, a cellulose acetate phthalate (CAP), a cellulose acetate succinate, hydroxypropyl methyl cellulose phthalate, a HPMCAS, a polyvinyl acetate phthalate (PVAP), a methyl methacrylate-methacrylic acid copolymer, a shellac, a cellulose acetate trimellitate, a sodium alginate, a zein, an enteric coating solution (an ethylcellulose, a medium chain triglycerides, an oleic acid, a sodium alginate, a stearic acid), or a combination thereof.

In some instances, a wall material coating can comprise an enteric coating, a time release coating, a pH dependent coating, a delayed release coating, an extended-release coating, or a combination thereof. The wall material can be biodegradable and biocompatible with the pharmaceutical ingredient. In some cases, the wall material can be biodegradable and biocompatible with a previously applied wall material.

In some cases, a microcapsule can be produced by dissolving, dispersing, or mixing the pharmaceutical ingredient in a solvent containing the shell material to produce a liquid suspension. In some cases, a multiple coated microcapsule can be produced by dissolving, dispersing, or mixing a previously microencapsulated pharmaceutical ingredient in a solvent containing a second shell material to produce a liquid suspension. For example, HPMCAS can be dissolved with ethanol and water and a pharmaceutical compound (e.g., the core) can be added the liquid suspension. In another example, an active ingredient encapsulated by HPMCAS can be dispersed with water and an enteric shell can be added to the liquid suspension. In some instances, the pharmaceutical compound may not dissolve in the liquid suspension. In some instances, the pharmaceutical compound may dissolve in the liquid suspension. In some instances, an encapsulated particle may not dissolve in the liquid suspension. In some instances, an encapsulated particle may dissolve in the liquid suspension. A liquid suspension can be dried with a spray drying technique described herein or by another method.

One or more layers of an enteric coating can be added to an active ingredient described herein by a microencapsulation process and/or fluidized system described herein to prevent it from dissolving until after it passes through the stomach. In some cases, two or more enteric coatings can be applied to an active ingredient. In some cases, a microencapsulated particle coating can release an active ingredient depending on the pH value within the gastrointestinal (GI) tract. The GI tract can have different pH values which can allow for pH dependent dosing in specific areas. For example, the pH of the stomach (acidic about 1.5-4.0 pH) is different from the pH of the small intestine (pH 4.0-7.0), and a pH microencapsulated particle coating can be used to dose areas of the GI tract with specific pH levels. In some embodiments, an enteric coating of a microencapsulated particle can be a polymer barrier that can be applied to the microencapsulated particle described herein to enable a controlled release. Bypassing the stomach can allow for more precise dosing and can enable the drug to achieve a higher bioavailability in the gastric tract. In some cases, these coatings or multiple layers of these coatings can be modified to deliver medicine from the mouth, all the way to the colon. In some cases, the technology can be applied to different microencapsulated layers of an active ingredient particle and utilize time-released, pH-controlled released, or a combination of both technologies to achieve the intended drug delivery. In some cases, one or more layers of a microcapsule shell can increase or decrease active ingredient release kinetics. In some cases, one or more layers of a microcapsule shell can increase or decrease bioavailability. In some cases, microencapsulation of an active ingredient as disclosed herein such as testosterone or a pharmaceutically acceptable salt thereof, can produce about: 5% to about 70%, 5% to about 10%, 5% to about 20%, 10% to about 30%, 15% to about 40%, 25% to about 40%, 10% to about 60%, or 20% to about 50% more bioavailability of the active ingredient or the salt thereof as compared to the active ingredient or the salt thereof that is not encapsulated when ingested by a subject.

In terms of thickness of an individual coating of a microencapsulated particle, the thickness can be more than, less than, or equal to about: 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some cases, the wall thickness can of an individual coating of a microencapsulated particle can range from about: 500 nm to about 1 μm, 1 μm to about 10 μm, 1 μm to about 5 μm, 2 μm to about 7 μm, 3 μm to about 8 μm, 5 μm to about 10 μm, 5 μm to about 15 μm, or 1 μm to about 30 μm.

In some instances, the wall thickness of a microencapsulated particle can increase by increasing the ratio of the wall material to the core material prior to spray drying. In some cases, the ratio of wall material to core material (weight/weight) can be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 60:1, 70:1, 80:1, 90:1, or 100:1.

In some cases, the ratio of the wall material to core material (weight/weight) can be about 10:1. In some instances, the wall thickness of a microencapsulated particle can increase by increasing the ratio of the wall material to a previously microencapsulated particle prior to spray drying. In some cases, the ratio of wall material to previously microencapsulated particle(weight/weight) can be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 60:1, 70:1, 80:1, 90:1, or 100:1.

In some embodiments, in a plurality of microencapsulated particles about: 1%, 5%, 10%, 15%, 20%, 25%, 30%, 35%, 40%, 45%, 50%, 55% 60%, 65%, 70%, 75%, 80%, 90%, 95%, 99% or 100% of the microencapsulated particles can comprise a core substantially encapsulated by a wall material. In some cases, in a plurality of microencapsulated particles about: 1% to about 50%, 1% to about 20%, 1% to about 10%, 5% to about 25%, 10% to about 40%, 10% to about 60%, 20% to about 70%, 20% to about 50%, 30% to about 80%, 40% to about 90%, 50% to about 75%, 60% to about 80%, 70% to about 90%, 75% to about 95%, 80% to about 90%, 80% to about 99%, 85% to about 100%, or 90% to about 100% of the microencapsulated particles can comprise a core substantially encapsulated by a wall material. In some cases, in a plurality of microencapsulated particles not all of the core material can be encapsulated by the wall material.

In certain instances, the formulations may be such that not all microencapsulated particles have the same number of coatings. This may be advantageous wherein delivery of an active ingredient may take place at multiple points along the digestive tract. In some cases, in a plurality of microencapsulated particles about: 1% to about 50%, 1% to about 20%, 1% to about 10%, 5% to about 25%, 10% to about 40%, 10% to about 60%, 20% to about 70%, 20% to about 50%, 30% to about 80%, 40% to about 90%, 50% to about 75%, 60% to about 80%, 70% to about 90%, 75% to about 95%, 80% to about 90%, 80% to about 99%, 85% to about 100%, or 90% to about 100% of the microencapsulated particles can comprise two or more coatings.

In some cases, the mixed sizes or mixed number of coatings can change the release time of the drug. For example, encapsulated particles that comprise an additional enteric coating with small sizes (e.g., about 20 μm to about 40 μm) can be readily absorbed from the intestines into the blood stream while larger enteric coated, encapsulated particles larger than about 60 μm can take longer to be absorbed into the blood stream. In some cases, particles with diameters of about 20 μm to about 40 μm can absorb faster than particles with diameters of about 50 μm to about 200 μm. In some embodiments, the particles with sizes of about 50 μm to about 200 μm can be mixed with particles with sizes of about 20 μm to about 40 μm. In some embodiments, the weight to weight ratio of the particles with diameters of about 70 μm to about 100 μm to the particles with sizes of about 30 μm to about 60 μm can range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10. In some embodiments, the weight to weight ratio of the particles with diameters of about 70 μm to about 100 μm to the particles with sizes of about 30 μm to about 60 μm can range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10. In some embodiments, the particles with larger sizes (about 70 μm to about 200 μm) can be mixed with particles with smaller sizes (about m to about 40 m). In some embodiments, the weight to weight ratio of the particles with larger sizes (about 70 μm to about 200 μm) to the particles with smaller sizes (about 20 μm to about 40 m) can be ranging from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10.

In some instances, the core diameter of a microencapsulated particle can be more than, less than, or equal to about: 100 nm (nanometer), 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some cases, the core diameter of a microencapsulated particle can range from about: 100 nm to about 250 nm, 100 nm to about 500 nm, 100 nm to about 1 μm, 500 nm to about 1 μm, 1 μm to about 10 μm, 1 μm to about 5 μm, 2 μm to about 7 μm, 3 μm to about 8 μm, 5 μm to about 10 μm, 5 μm to about 15 μm, or 1 μm to about 30 μm. In some instances, the core can comprise about: 1%, 5%, 10%, 15%, 20%, 25%, 30%, 35%, 40%, 45%, 50%, 55%, 60%, 65%, 70%, 75%, 80%, 90%, 95% or 99% of the total microcapsule content (e.g., total weight of the core and wall material). In some instances, the core can comprise about: 1% to about 50%, 1% to about 20%, 1% to about 10%, 5% to about 25%, 10% to about 40%, 10% to about 60%, 20% to about 70%, 20% to about 50%, 30% to about 80%, 40% to about 90%, 50% to about 75%, or 1% to about 99% of the total microcapsule content.

### Method of Making the Powdery Pharmaceutical Composition

In some cases, methods of making a pharmaceutical composition may comprise creating particles by the methods described herein. In some cases, particles may comprise an excipient (e.g., a pharmaceutically acceptable excipient), an active ingredient, or both. In some aspects, a method of making a powdery pharmaceutical composition, may comprise mixing, in a mixer, particles of a pharmaceutically acceptable excipient; and particles comprising an active ingredient or a pharmaceutically acceptable salt thereof. In some cases, the particles comprising an active ingredient may be microencapsulated. For example, particles of a hormone receptor agonist may be microencapsulated with trehalose and/or optionally a HPMC or HPMCAS coating. In some instances, at least a portion of the particles of the pharmaceutically acceptable excipient may have a particle diameter ranging from about 50 micrometers to about 200 micrometers, as measured by a particle size analyzer using laser diffraction. In some cases, particles comprising the active ingredient may have a particle diameter ranging from about 500 nanometers to about 15 micrometers, or 1 micrometer to about 20 micrometers, as measured by a particle size analyzer using laser diffraction.

In some aspects, a method of making the powdery pharmaceutical composition may comprise particles wherein at least a portion of the particles of the active ingredient or a pharmaceutically acceptable salt thereof may be made by a spray drying process.

In some aspects, the spray drying process may comprise: atomizing liquid droplets comprising the active ingredient or the pharmaceutically acceptable salt thereof, drying the droplets from particles, recovering the particles, or any combination thereof.

In some aspects, a spray drying manufacturing system may comprise a closed spray dryer container which receives the solution comprising a drug dissolved or mixed in a suitable solvent (aqueous or solvent based). In some cases, a solvent may comprise alcohol, ethanol, dimethylformamide (DMF), dimethyl sulfoxide (DMSO), a polar organic solvent, an organic solvent, or any combination thereof. In some aspects, the solution then enters the particle formation chamber which may be connected to an atomizer located at the top of the chamber. In some aspects, the atomizer may be a two component or rotary nozzle type that distributes the solution into fine droplets controlled by the atomizer pressure. In some aspects, this atomization gas may be an inert gas. As used herein, “inert gas” may refer to a non-reactive gas, or a gas that does not undergo chemical reactions under a set of given conditions. Inert gases may be generally used to avoid unwanted chemical reactions degrading a sample, or to prevent bacterial growth. These undesirable chemical reactions may often be oxidation and hydrolysis reactions with the oxygen and moisture in air. The term “inert gas” may be context-dependent because several of the noble gases, which have been historically referred to as the inert gases, may be made to react under certain conditions. In some aspects, inert gas may be air, nitrogen, carbon dioxide or any combination thereof. In some aspects, the atomized droplets go through a hot gas drying chamber to produce uniform fine particles that maintain a tight particle size distribution following liquid evaporation. In some cases, the solid particle forms and falls to the bottom of the drying chamber. In some instances, the balance between temperature, flow rate, and droplet size may controls the drying process. In some aspects, the powder may be recovered from the exhaust gas using a cyclone or a bag filter. In some aspects, particle size may be validated by a Malvern particle analyzer prior to blending with an excipient carrier. In some aspects, the active powder (e.g., the powdery pharmaceutical composition) may be blended with an excipient carrier (lactose) product in a Patterson Kelly (PK Blender) and the blended powder may be fed to a hopper. In some aspects, from the hopper, the dry powder may be placed into a Size 3 hypromellose capsule, by a Bosch Encapsulator machine. In some cases, the dry powder may be placed into any capsule of any size. For example, the dry powder may be placed into a size 000, 00, 0, 1, 2, 3, or a 4-size capsule.

The moisture level of the powder after spray drying may be below about 10%. In some aspects, the moisture level may be below about 15%, about 14%, about 13%, about 12%, about 11%, about 10%, about 9%, about 8%, about 7%, about 6%, about 5%, about 4%, about 3%, about 2%, or about 1%.

In some embodiments, the method of making a composition can comprise formulating the particles described herein into a capsule-in-capsule composition (e.g. a pharmaceutical composition). In some cases, particles can comprise an excipient, an active ingredient, or both. In some cases, particles can comprise a carrier, an active ingredient, or both. In some cases, particles can comprise a diluent, an active ingredient, or both. In some embodiments, a capsule-in-capsule formulation can be in unit dose form. In some instances, the particles can be at least partially encapsulated by a coating material. In some instances, the particles at least partially encapsulated by the coating material can be spray dried. In some cases, the particles can be at least partially surrounded by a first capsule, a second capsule, or both. In some instances, the first capsule can be surrounded by a second capsule to create a capsule-in-capsule, capsule. In some cases, a capsule can comprise a capsule coating. In some cases, a capsule coating can at least partially control capsule ingredient release.

In some embodiments, a final product can be a capsule-in-capsule. In some cases, the final product can be a capsule (e.g., a second capsule) that surrounds an active ingredient (e.g., a hormone receptor agonist) and separately an inner capsule (e.g., the first capsule), which can contain its own active ingredient. In some instances, a capsule can contain more than one active ingredient. In some instances, a capsule can contain more than one inner capsule. For example, a capsule can comprise 1, 2, 3, 4, 5, 6, 7, 8, 9, 10 or more inner capsules. In some cases, an inner capsule can comprise a capsule. For example, an outer capsule can surround a first inner capsule and the first inner capsule can surround a second inner capsule. In some cases, an active ingredient can comprise a steroid, a human growth hormone receptor agonist, or a glucagon-like peptide 1 receptor agonist. In some cases, the active ingredient can be microencapsulated and spray dried. In some cases, the active ingredient can be spray dried but not microencapsulated.

The process described herein can include the following manufacturing stages. The active ingredient of the first capsule and the second capsule can be microencapsulated and spray dried using the methods described herein. In some embodiments, the active ingredients can be independently blended with an excipient. In some cases, the active ingredients may not be blended with an excipient. In some cases, the active ingredient of the first capsule can then be added to the first capsule and the first capsule can be banded using the methods described herein. In some cases, after the first capsule is banded, a capsule coating (e.g. an enteric, pH dependent, time release, or combination release) can be applied to the first capsule. In some cases, the active ingredient of the second capsule can then be added to the second capsule and the first capsule can be placed into the second capsule. The second capsule can be banded, and a capsule coating can be applied to the second capsule.

In some embodiments, a capsule can further comprise a capsule coating. In some cases, a capsule coating can be added to a capsule to further improve stability (light protection, moisture and gas barrier), facilitate administration, or modify the composition release behavior from the dosage form. A capsule coating may be used to enable the immediate release of the composition, delay the release of the composition (such as in enteric coatings), or sustain the release of the composition over extended periods of time. In some instances, a capsule coating can comprise a film coating, a gelatin coating, or both. In some instances, a capsule coating can comprise an enteric coating, a time release coating, a pH dependent coating, a delayed release coating, an extended-release coating, or a combination thereof. For example, an enteric coating can be added to a capsule to prevent it from dissolving until after it passes through the stomach. In some cases, the composition can release depending on the pH value within the gastrointestinal (GI) tract. The GI tract can have different pH values which can allow for pH dependent dosing in specific areas. For example, the pH of the stomach (acidic about 1.5-4.0 pH) is different from the pH of the small intestine (pH 4.0-7.0), and a pH coating can be used to dose areas of the GI tract with specific pH levels. In some embodiments, an enteric coating of a capsule can be a polymer barrier that can be applied to the capsules described herein to enable a controlled release. Bypassing the stomach can allow for more precise dosing and can enable the drug to achieve a higher bioavailability in the gastric tract. In some cases, these coatings can be modified to deliver medicine from the mouth, all the way to the colon. In some cases, the technology can be applied to the outer (e.g. the second capsule) and the inner (e.g. the first capsule) capsule in the capsule-in-capsule technology and utilize time-released, pH-controlled released, or a combination of both technologies to achieve the intended drug delivery. In some instances, an enteric coating can be applied to multiple capsules, for example to an inner capsule and to an outer capsule and to provide delayed release of both capsules. In some cases, a capsule coating can provide a color, mask a bitter taste, or both. In some cases, a capsule coating can comprise polymers, plasticizers, pigments, opacifiers, glidants, binders, anti-tacking agents, anti-foaming mechanisms, surfactants, fillers, and extenders.

In certain instances, the pharmaceutical formulation does not include a surfactant. In other instances, the pharmaceutical formulation includes a surfactant. Surfactants may be nonionic surfactants, anionic surfactants, cationic surfactants, amphoteric surfactants, and tryglycerides. Examples of nonionic surfactants include but are not limited to: aocamide monoethanolamine (Cocamide MEA), cocamide diethanolamine (Cocamide DEA), fatty alcohol ethoxylates, Tween 20, Tween 40, Tween 60, and Tween 80. Examples of anionic surfactants include but are not limited to: sodium lauryl sulfate (SLS), sodium laureth sulfate (SLES), ammonium lauryl sulfate (ALS), ammonium laureth sulfate (ALES), sodium stearate, and potassium cocoate. Examples of cationic surfactants include but are not limited to: tetramethylammonium chloride, tetrapropylammonium hydroxide, tetrabutylammonium bromide, tetrabutylammonium hydroxide, trimethylphenylammonium chloride, benzyltrimethylammonium chloride, benzyltriethylammonium chloride, and benzyltributylammonium chloride. Examples of amphoteric surfactants include but are not limited to: alkylamidopropylamine N-oxide (APAO), alkyldimethylamine N-oxide (AO), alkylbetaine (Bt) and alkylamidopropylbetaine (APB).

In some embodiments, an enteric coating can comprise a polymer. In some cases, an enteric coating can comprise a methyl acrylate-methacrylic acid copolymer, a cellulose acetate phthalate, methyl methacrylate (MMA), a cellulose acetate succinate, a hydroxypropyl methyl cellulose phthalate, a hydroxypropyl methyl cellulose acetate succinate (hypromellose acetate succinate), a polyvinyl acetate phthalate, a methyl methacrylate-methacrylic acid copolymers, a shellac, a cellulose acetate trimellitate, a sodium alginate, a zein, an ethylcellulose, a medium chain triglycerides, an oleic acid, a stearic acid or any combination thereof.

In some embodiments, a capsule can be configured (for example with a capsule coating) to at least partially release an active ingredient in: the mouth, the esophagus, the small intestine, the duodenum, the jejunum, the ileum, the cecum, the colon, the ascending colon, the traverse colon, the descending colon, the sigmoid colon, the rectum, the anus, or any combination thereof.

In some cases, a composition can comprise a mixture of particles described herein. In some instances, at least a portion of an excipient and at least a portion of the particles comprising an active ingredient, can comprise a mixture or a formulation.

### Microencapsulation

In some embodiments, encapsulation may comprise microencapsulation. Microencapsulation may be a process in which a microcapsule may be created as a small sphere or multi-sphere in one core with a matrix wall around it. The pharmaceutical ingredient inside the microcapsule may be called a fill. In some cases, a fill may be a liquid, an oil, a solid or any combination thereof. The wall around the fill (“or core”) may be referred to as a shell, a coating, or a membrane. Microcapsules may have a diameter as small as 1.0 micron in size to about 10.0 micron in size or about 1.0 micron to about 5.0 microns in size. In some cases, the small size may provide a pharmaceutical ingredient a large surface area to be available for absorption, release, transfer, or any combination thereof. In some cases, microencapsulation may at least partially prevent inhalation of an active ingredient comprising the form of an unencapsulated crystal. For example, microencapsulation may at least partially prevent inhalation of unencapsulated crystals comprising a hormone receptor agonist. In some instances, unencapsulated crystals such as hormone receptor agonist crystals may cause irritation of the respiratory tract of a subject during inhalation. The irritation may be caused by crystal geometry and structure. For example, a crystal may have sharp angles and edges that may cause irritation, damage or both of the respiratory tract during inhalation. In some instances, crystal geometry and structure may be controlled by the spray drying process. Microencapsulation may generate crystals with amorphous structure. In some instances, an amorphous crystal may lack sharp edges and angles. In some cases, an amorphous crystal may have a rounded edge. In some instances, an amorphous crystal may have increased bioavailability.

In some instances, an agonist described herein comprised in an oil may be microencapsulated with compatible diluents to protect the oil from oxidation and provide a longer shelf life than the unprotected pharmaceutical composition. Similarly, a hormone receptor agonist may be encapsulated to provide a longer shelf life. The diluents may be aqueous, or solvent based and use animal or plant materials. In some cases, the diluent may comprise alcohols: e.g., ethanol, butanol, 2-ethylhexanol, isobutanol, isopropanol, methanol, propanol, propylene glycol; ketones: e.g., acetone, methyl ethyl ketone, methyl isobutyl ketone, methyl isopropyl ketone, mesityl oxide, trichloroethylene; halogenated solvents: e.g., ethylene bromide, chloroform, ethylene chloride, dichloromethane, tetrachloroethylene, carbon tetrachloride; amides: e.g., dimethylformamide; ethers: e.g., 1,4-dioxane, butyl ether, ethyl ether, di-isopropyl ether, tetrahydrofuran, tert-butyl methyl ether; sulfur containing solvent: e.g., dimethyl sulfoxide; amines: e.g., pyridine; nitriles: e.g., acetonitrile; esters: e.g., ethyl acetate; aliphatic hydrocarbons: e.g., cyclohexane hexane; aromatic hydrocarbons: e.g., toluene xylene; water or any combinations thereof. In some cases, the diluent may comprise benzene, carbon tetrachloride, 1,2-dichloroethane, 1,1-dichloroethene, 1,1,1-trichloroethane, acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethylene glycol, formamide, hexane, methanol, 2-methoxyethanol, methylbutylketone, methylcyclohexane, n-methylpyrrolidone, nitromethane, pyridine, sulfolane, tetralin, toluene, 1,1,2-trichloroethylene, xylene or any combinations thereof.

The core active ingredient may be microencapsulated with an amphipathic molecule that has both a polar end (‘hydrophilic”) and non-polar end (“hydrophobic”). In some cases, a hydrophilic end of an amphipathic molecule may interact with core material. In some cases, a hydrophobic end of an amphipathic molecule may interact with core material. This hydrophilic and hydrophobic structure may enable the molecule to microencapsulate an active ingredient and form a microsphere. In some instances, the microencapsulated particle may have a hydrophilic exterior and a hydrophobic interior. In some instances, the microencapsulated particle may have a hydrophobic exterior and a hydrophilic interior. The microencapsulation process may coat the active ingredient, which is the core, by the amphipathic encapsulating agent, which is the wall material, so that the active ingredient is at least partially surrounded by a wall of the amphipathic material. For example, hydroxypropyl methylcellulose acetate succinate (HPMCAS) may be an amphipathic molecule used to coat a hormone receptor agonist. The microencapsulation blend may be a spray dried dispersion, that may be fed into a spray dry system to create a hard-outer coating on the microcapsules.

The wall material may form a film that is cohesive with the core active ingredient. A wide variety of coating materials are available for encapsulation, e.g., traditional coating materials like inert polymers and pH sensitive ones as carboxylate and amino derivatives, which swell or dissolve according to the degree of cross-linking; some innovative coating polymers have also been developed for applications particularly among the bioadhesives and mucoadhesives. In some cases, the coating material may be hydrophilic polymers, hydrophobic polymers or a combination of both. In some cases, a microcapsule shell may comprise an amphipathic molecule. In some cases, the coating material may be a gelatin, a polyvinyl alcohol, an ethyl cellulose, a cellulose acetate phthalate or a styrene maleic anhydride. In some instances, the coating material may not react with the pharmaceutical ingredient. In some cases, a microcapsule shell may comprise trehalose, a hydroxypropyl methylcellulose (“HPMC”), a hydroxypropyl methylcellulose acetate succinate (“HPMCAS”), a cyclodextrin, a maltodextrin, a povidone, a copovidone and others. In some instances, a microcapsule shell may comprise HPMCAS-LG, HPMCAS-MG, HPMCAS-HG or HPMC-P or a combination thereof. In some instances, a microcapsule shell may comprise a different grade of HPMC or HPMCAS. For example, a microcapsule shell may comprise an E5, an E50, or a K4M grade of HPMC. In another example, a microcapsule shell may comprise a L, a M, or an H grade of HPMCAS. In some cases, a microcapsule shell may comprise trehalose and/or a HPMCAS. In some cases, a microcapsule shell may comprise a gelatin, a cornstarch, a polyvinylpyrrolidone (PVP), an oligosaccharide, a starch, a cellulose, a glocogen, a long chain sugar or any combination thereof. In some cases, a microcapsule shell may comprise FDKP (fumaryl diketopiperazine). In some cases, a microcapsule shell can comprise 1,2-distearoyl-sn-glycero-3-phosphocholine (DSPC).

In some cases, a microcapsule shell can contain a weight to weight ratio of the DSPC, and the FDKP of about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 55:1, 60:1, 65:1, 70:1, 75:1, 80:1, 85:1, 90:1, 95:1, or 100:1 (DSPC to FDKP). In some cases, a microcapsule shell can contain a weight to weight ratio of the FDKP, and the DSPC of about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 55:1, 60:1, 65:1, 70:1, 75:1, 80:1, 85:1, 90:1, 95:1, or 100:1 (FDKP to DSPC).

In some cases, a microcapsule shell may comprise a fatty acid, a liposome, an amino acid, a natural oil and a sugar, a trehalose, a dextran, a natural oil, a synthetic oil or a combination thereof. In some cases, a sugar can comprise a dextrose, a fructose, a galactose, a glucose, a lactose, a maltose, a sucrose, a salt of any of these, or any combination thereof. In some instances, an amino acid may comprise a glutamic acid, an aspartic acid, a lysine, a tryptophan, a tyrosine, a methionine or a combination thereof. In some cases, a fatty acid may comprise a polyunsaturated fatty acid, an essential fatty acid, a conjugated fatty acid, a short chain fatty acid, a medium chain fatty acid, a long chain fatty acid, a very long chain fatty acid, a saturated fatty acid, an unsaturated fatty acid, a monounsaturated fat, or any combination thereof. In some cases, a fatty acid may comprise an omega-3 fatty acid, an omega-5 fatty acid, an omega-6 fatty acid, an omega-7 fatty acid, an omega-9 fatty acid, an omega-10 fatty acid, an omega-11 fatty acid, an omega-12 fatty acid, or a combination thereof. In some cases, a natural oil may comprise soybean oil, a vegetable oil, a food oil, evening primrose oil, borage oil, blackcurrant seed oil, flax or linseed oil, rapeseed or canola oil, corn oil, almond oil, avocado oil, Brazil nut oil, canola oil, cashew oil, chia seed oil, cocoa butter oil, coconut oil, corn oil, cottonseed oil, flaxseed/linseed oil, grape seed oil, hemp seed oil, Vigna mungo oil, mustard oil, olive oil, palm oil, peanut oil, pecan oil, perilla oil, rice bran oil, safflower oil, sesame oil, soybean oil, walnut oil, sunflower oil, cottonseed oil, palm oil, or a combination thereof. In some cases, a microcapsule shell may increase or decrease active ingredient release kinetics. In some cases, a microcapsule shell may increase or decrease bioavailability. In some cases, microencapsulation of a hormone receptor agonist, may produce about: 5% to about 70%, 5% to about 10%, 5% to about 20%, 10% to about 30%, 15% to about 40%, 25% to about 40%, 10% to about 60%, or about 20% to about 50% more bioavailability of the hormone receptor agonist thereof as compared to a hormone receptor agonist that is not encapsulated when inhaled by a subject. The wall material may be biodegradable and biocompatible with the pharmaceutical ingredient. In some cases, a microcapsule may be produced by dissolving or mixing the pharmaceutical ingredient in a solvent containing the shell material to produce a liquid suspension. For example, HPMCAS may be dissolved with ethanol and water and a pharmaceutical compound may be added the liquid suspension. In some instances, the pharmaceutical compound may not dissolve in the liquid suspension. In some instances, the pharmaceutical compound may dissolve in the liquid suspension. The liquid suspension may be dried with a spray drying technique described herein or by another method.

In some cases, the average wall thickness may of a microencapsulated particle can be more than, less than, or equal to about: 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some cases, the wall thickness may of a microencapsulated particle may range from about: 1 μm to about 10 μm, 1 μm to about 5 μm, 2 μm to about 7 μm, 3 μm to about 8 μm, 5 μm to about 10 μm, 5 μm to about 15 μm, or 1 μm to about 30 μm. In some instances, the wall thickness of a microencapsulated particle may increase by increasing the ratio of the wall material to the core material prior to spray drying. In some cases, the ratio of wall material to core material (weight/weight) may be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 60:1, 70:1, 80:1, 90:1, or 100:1. In some cases, the ratio of the wall material to core material (weight/weight) may be about 10:1. In some cases, a pharmaceutical composition herein can comprise particles of a pharmaceutical composition that have different wall thicknesses. For example, a metformin particle herein can be microencapsulated and the microencapsulated particles that have 1, 2, 3, 4 or more different wall thicknesses. In some cases, the different sizes of the wall thickness can alter the drug release of a particle. For example, a microencapsulated particle with a thicker wall thickness will have a delayed drug release as compared to a microencapsulated particle with a thinner wall thickness.

In some aspects, in a plurality of microencapsulated particles about: 1%, 5%, 10%, 15%, 20%, 25%, 30%, 35%, 40%, 45%, 50%, 55%, 60%, 65%, 70%, 75%, 80%, 90%, 95%, 99% or 100% of the microencapsulated particles may comprise a core substantially encapsulated by a wall material. In some cases, in a plurality of microencapsulated particles about: 1% to about 50%, 1% to about 20%, 1% to about 10%, 5% to about 25%, 10% to about 40%, 10% to about 60%, 20% to about 70%, 20% to about 50%, 30% to about 80%, 40% to about 90%, 50% to about 75%, 60% to about 80%, 70% to about 90%, 75% to about 95%, 80% to about 90%, 80% to about 99%, 85% to about 100%, or 90% to about 100% of the microencapsulated particles may comprise a core substantially encapsulated by a wall material. In some cases, in a plurality of microencapsulated particles not all of the core material may be encapsulated by the wall material.

In some aspects, microencapsulated particles have a mean, a median, or a mode particle diameter of less than about: 500 nm, 1 μm, 2 μm, 3 μm, 4 μm, 5 μm, 6 μm, 7 μm, 8 μm, 9 μm, 10 μm, 11 μm, 12 μm, 13 μm, 14 μm, or 15 μm. In some aspects, microencapsulated particles have a mean, a median, or a mode particle diameter of more than, or equal to about: 500 nm, 1 μm, 2 μm, 3 μm, 4 μm, 5 μm, 6 μm, 7 μm, 8 μm, 9 μm, 10 μm, 11 μm, 12 μm, 13 μm, 14 μm, or 15 μm. In some aspects, microencapsulated particles have a mean, a median, or a mode particle diameter ranging from about: 500 nm to about 5 μm, 1 μm to about 10 μm, 1 μm to about 5 μm, 2 μm to about 7 μm, 3 μm to about 8 μm, 5 μm to about 10 μm, or 5 μm to about 15 μm.

In some embodiments, microencapsulated particles have a mean, a median, or a mode particle diameter of less than about: 500 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 jm, 91 μm, 92 μm, 93 μm, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, 110 μm, 111 μm, 112 μm, 113 μm, 114 μm, 115 μm, 116 μm, 117 μm, 118 μm, 119 μm, 120 μm, 121 μm, 122 μm, 123 μm, 124 μm, 125 μm, 126 μm, 127 μm, 128 μm, 129 μm, 130 μm, 131 μm, 132 μm, 133 μm, 134 μm, 135 μm, 136 μm, 137 μm, 138 μm, 139 μm, 140 μm, 141 μm, 142 μm, 143 μm, 144 μm, 145 μm, 146 μm, 147 μm, 148 μm, 149 μm, 150 μm, 151 μm, 152 μm, 153 μm, 154 μm, 155 μm, 156 μm, 157 μm, 158 μm, 159 μm, 160 μm, 161 μm, 162 μm, 163 μm, 164 μm, 165 μm, 166 μm, 167 μm, 168 μm, 169 μm, 170 μm, 171 μm, 172 μm, 173 μm, 174 μm, 175 μm, 176 μm, 177 μm, 178 μm, 179 μm, 180 μm, 181 μm, 182 μm, 183 μm, 184 μm, 185 μm, 186 μm, 187 jm, 188 μm, 189 μm, 190 μm, 191 μm, 192 μm, 193 μm, 194 μm, 195 μm, 196 μm, 197 μm, 198 μm, 199 μm, or 200 μm. In some embodiments, microencapsulated particles have a mean, a median, or a mode particle diameter of more than, or equal to about: 500 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, 110 μm, 111 μm, 112 μm, 113 μm, 114 μm, 115 μm, 116 μm, 117 μm, 118 μm, 119 μm, 120 μm, 121 μm, 122 μm, 123 μm, 124 μm, 125 μm, 126 μm, 127 μm, 128 μm, 129 μm, 130 μm, 131 μm, 132 μm, 133 μm, 134 μm, 135 μm, 136 μm, 137 μm, 138 μm, 139 μm, 140 μm, 141 μm, 142 μm, 143 μm, 144 μm, 145 μm, 146 μm, 147 μm, 148 μm, 149 μm, 150 μm, 151 μm, 152 μm, 153 μm, 154 μm, 155 μm, 156 μm, 157 μm, 158 μm, 159 μm, 160 μm, 161 μm, 162 μm, 163 μm, 164 μm, 165 μm, 166 μm, 167 μm, 168 μm, 169 μm, 170 μm, 171 μm, 172 μm, 173 μm, 174 μm, 175 μm, 176 μm, 177 μm, 178 μm, 179 μm, 180 μm, 181 μm, 182 μm, 183 μm, 184 μm, 185 μm, 186 μm, 187 μm, 188 μm, 189 μm, 190 μm, 191 μm, 192 μm, 193 μm, 194 μm, 195 μm, 196 μm, 197 μm, 198 μm, 199 μm, or 200 μm. In some embodiments, microencapsulated particles have a mean, a median, or a mode particle diameter ranging from about: 500 nm to about 5 μm, 1 μm to about 10 μm, 1 μm to about 5 μm, 2 μm to about 7 μm, 3 μm to about 8 μm, 5 μm to about 10 μm, 5 μm to about 15 μm, 10 μm to about 50 μm, 20 μm to about 100 μm, 30 μm to about 70, 50 μm to about 150, 70 μm to about 100 μm, 70 μm to about 140, 100 μm to about 180, or 120 μm to about 200 μm. In some cases, microencapsulated particles in a capsule in capsule formulation can be larger than microencapsulated particles for use in a dry powdered inhalable formulation.

The core material may be the material over which coating has to be applied to serve the specific purpose. Core material may be in form of solids or droplets of liquids and dispersions. In some cases, core material may comprise a hormone receptor agonist. In some cases, core material may comprise another hormone receptor agonist or a salt thereof. The composition of core material may vary and thus furnish definite flexibility and allow effectual design and development of the desired microcapsule properties. A substance may be microencapsulated for a number of reasons. Examples may include protection of reactive material from their environment, safe and convenient handling of the materials which may be otherwise toxic or noxious, taste masking, means for controlled or modified release properties means of handling liquids as solids, preparation of free flow powders and in modification of physical properties of the drug. For example, encapsulation may improve solubility and dissolution and therefore increase bioavailability of an active ingredient such as a hormone receptor agonist. Microencapsulation may be used to increase the stability, improve the handling properties of compounds, facilitate higher bioavailability when reconstituted or administered, or any combination thereof. For example, microencapsulated a hormone receptor agonist may not require refrigeration and may not lose efficacy when exposed to a high temperature for a period of time. In some cases, the shelf life of encapsulated a hormone receptor agonist may be extended from about: 4, 5, 6, 7, 8, 9, 10, 11, 12, 13, 14, 15, 16, 17, 18, 19, 20, 21, 22, 23, 24 or more months as compared to a liquid formation comprising a hormone receptor agonist. In some instances, inhalable an hormone receptor agonist may not require a needle for administration.

In some instances, the core diameter of a microencapsulated particle may be more than, less than, or equal to about: 100 nm (nanometer), 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some cases, the core diameter of a microencapsulated particle may range from about: 100 nm to about 250 nm, 100 nm to about 500 nm, 100 nm to about 1 μm, 500 nm to about 1 μm, 1 μm to about 10 μm, 1 μm to about 5 μm, 2 μm to about 7 μm, 3 μm to about 8 μm, 5 μm to about 10 μm, 5 μm to about 15 μm, or 1 μm to about 30 μm. In some instances, the core may comprise about: 1%, 5%, 10%, 15%, 20%, 25%, 30%, 35%, 40%, 45%, 50%, 55%, 60%, 65%, 70%, 75%, 80%, 90%, 95% or 99% of the total microcapsule content (e.g., total weight of the core and wall material). In some instances, the core may comprise about: 1% to about 50%, 1% to about 20%, 1% to about 10%, 5% to about 25%, 10% to about 40%, 10% to about 60%, 20% to about 70%, 20% to about 50%, 30% to about 80%, 40% to about 90%, 50% to about 75%, or 1% to about 99% of the total microcapsule content.

In some aspects, a method of microencapsulation may comprise at least partially dissolving the coating material (e.g., HPMC or HPMCAS or trehalose) in a solvent such as ethanol and water mix. In some cases, particles of a hormone receptor agonist may be added to the solution of the coating material and the solvent to create a suspension of the particles of a hormone receptor agonist and the coating material dissolved in the solvent. In some instances, the hormone receptor agonist may not dissolve in the suspension and may remain in suspension. In some instances, the hormone receptor agonist may dissolve in the suspension. The suspension may be mixed to an at least partially uniform mixture and spray dried. The coating may at least partially encapsulate the psilocybin or salt thereof. In some cases, the hormone receptor agonist may be amorphous. In some cases, the encapsulation of the hormone receptor agonist may be a spherical, round, oval, or any shape structure.

### Methods of Making

Also disclosed herein are methods of making the powdery pharmaceutical composition to select sizes. In some cases, the formulating of a composition can comprising mixing particles of an excipient and an active agent. In some cases, the formulating of a composition can comprise making particles of a specific size.

In some aspects, the method of making the powdery pharmaceutical composition may comprise mixing the particles described herein with an excipient. In some instances, at least a portion of the particles of a pharmaceutically acceptable excipient may have a particle diameter ranging from about 50 micrometers to about 200 micrometers, as measured by a particle size analyzer using laser diffraction. In some cases, at least a portion of the particles comprising an encapsulated hormone receptor agonist may have a particle diameter ranging from about 500 nanometers to about 15 micrometers, or about 1 micrometer to about 20 micrometers, as measured by a particle size analyzer using laser diffraction.

In some aspects, at least a portion of the particles of a pharmaceutically acceptable excipient may have a particle diameter ranging from about: 30 μm (micrometers) to about 60 μm, 50 μm, to about 200 μm, 60 μm to about 80 μm, 70 μm to about 100 μm. 90 μm to about 130 μm, 110 μm to about 150 μm, 130 μm to about 180 μm, 150 μm to about 200 μm, 190 μm to about 250 μm, or 200 μm to about 400 μm. In some cases, at least a portion of particles of a pharmaceutically acceptable excipient may have a particle diameter of more than, or equal to about: 30 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, or 400 μm. In some cases, at least a portion of particles of a pharmaceutically acceptable excipient may have a particle diameter of less than about: 30 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, or 400 μm. In some cases, the particles of a pharmaceutically acceptable excipient may range from about 50 μm to about 100 μm, which may be preferred when inhaled or administered intranasally for deposit on the oropharynx. In some instances, particle size as may comprise the diameter, the radius, or length of a particle. In some instances, particle size may be a measure of the mean, the median or the mode of a plurality of particles.

Regarding particle size, in certain instances, a particle may be measured by its aerodynamic diameter. The aerodynamic diameter (Dₐₑ) is a spherical equivalent diameter and derives from the equivalence between the inhaled particle and a sphere of unit density (ρo) undergoing sedimentation at the same rate as per the following formula:

Dₐₑ=Dᵥ √(ρ/χρo) where Dv is the volume-equivalent diameter, p is the particle density and x is the shape factor. Hence, the aerodynamic behavior depends on particle geometry, density and volume diameter: a small spherical particle with a high density will behave aerodynamically as a bigger particle, being poorly transported in the lower airways. The Dₐₑ can be improved reducing the volume diameter and the density or increasing the shape factor of the particles, by means of different processes.

In the case of a pharmaceutical formulation being administered as an aerosol. An “aerosol” may be a suspension of a suspension of liquid and solid particles produced by an aerosol generator such as a small-volume nebulizer (SVN), a pressurized metered-dose inhaler (pMDI), or a dry-powder inhaler (DPI). An “aerosol deposition” may be a process of aerosol particles depositing on absorbing surfaces. An “aerosol generator” may be a device used for producing aerosol particles. An “aerosol output” may be mass of medication exiting an aerosol generator. An “aerosol therapy” may be a delivery of a solid or liquid aerosol.

In some aspects, i) particles of an active ingredient (e.g. a hormone receptor agonist) or a pharmaceutically acceptable salt thereof or ii) at least partially encapsulated particles comprising an active ingredient or a pharmaceutically acceptable salt thereof may have particle diameters ranging from about: 100 nm (nanometer) to about 500 nm, 300 nm to about 800 nm, 700 nm to about 1.2 μm, 1 μm to about 3 μm, 2 μm to about 4 μm, 3 μm to about 6 μm, 5 μm to about 8 μm, 6 μm to about 9 μm, 7 μm to about 10 μm, 8 μm to about 11 μm, 9 μm to about 13 μm, 10 μm to about 15 μm, 12 μm to about 20 μm, 14 μm to about 25 μm, or 18 μm to about 30 μm. In some cases, i) particles of an active ingredient or a pharmaceutically acceptable salt thereof or ii) at least partially encapsulated particles comprising an active ingredient or a pharmaceutically acceptable salt thereof may have a particle diameter of less than about: 30 nm, 50 nm, 60 nm, 70 nm, 80 nm, 90 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some cases i) particles of an active ingredient or a pharmaceutically acceptable salt thereof or ii) at least partially encapsulated particles comprising an active ingredient or a pharmaceutically acceptable salt thereof may have a particle diameter of more than, or equal to about: 30 nm, 50 nm, 60 nm, 70 nm, 80 nm, 90 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some cases, i) particles of an active ingredient or a pharmaceutically acceptable salt thereof or ii) at least partially encapsulated particles comprising an active ingredient or a pharmaceutically acceptable salt thereof may be in about 1 μm to about 5 μm, which may be preferred when inhaled or administered intranasally for absorption into lung alveoli.

In some aspects, a particles or compositions described herein may have a tap density of more than about: 0.1 grams/centimeter (g/cm³), 0.2 g/cm³, 0.3 g/cm³, 0.4 g/cm³, 0.5 g/cm³, 0.6 g/cm³, 0.7 g/cm³, 0.8 g/cm³, 0.9 g/cm³, 1.0 g/cm³, 1.1 g/cm³, or 1.2 g/cm³. In some aspects, particles described herein may have a tap density of less than about: 0.1 g/cm³, 0.2 g/cm³, 0.3 g/cm³, 0.4 g/cm³, 0.5 g/cm³, 0.6 g/cm³, 0.7 g/cm³, 0.8 g/cm³, 0.9 g/cm³, 1.0 g/cm³, 1.1 g/cm³, or 1.2 g/cm³. In some cases, particles or compositions described herein may have a tap density of more than about 0.6 g/cm³ or 0.7 g/cm³. In some cases, particles or compositions described herein may have a tap density of about 0.6 g/cm³ or 0.7 g/cm³. In some cases, tap density of a powder may be the ratio of the mass of the powder to the volume occupied by the powder after it has been tapped for a defined period of time. In some cases, tap density may be a measure of the envelope mass density characterizing a particle. The envelope mass density of a particle of a statistically isotropic shape may be defined as the mass of the particle divided by the minimum sphere envelope volume within which it may be enclosed. Features which may contribute to low tap density include irregular surface texture, porous structure or a combination thereof. Tap density may be measured by using instruments known to those skilled in the art such as the Dual Platform Microprocessor Controlled Tap Density Tester (Vankel, N.C.) or a GeoPyc™ instrument (Micrometrics Instrument Corp., Norcross, Ga.).

In some aspects, particles of an active ingredient or a pharmaceutically acceptable salt thereof may be mixed in sizes. In some cases, the mixed sizes may change the release time of the drug. For example, particles with small sizes (e.g., about 1 μm to about 5 μm) may be readily absorbed into the blood stream while particles larger than about 10 μm may take longer to be absorbed into the blood stream. In some cases, particles with diameters of about 1 μm to about 10 μm may be inhaled into the lung while larger particles may be deposited onto the oropharynx. In some cases, particles with diameters of about 1 μm to about 5 μm may absorb faster than particles with diameters of about 7 μm to about 10 μm. In some aspects, the particles with sizes of about 7 μm to about 10 μm may be mixed with particles with sizes of about 1 μm to about 5 μm. In some aspects, the weight to weight ratio of the particles with diameters of about 7 μm to about 10 μm to the particles with sizes of about 1 μm to about 5 μm may range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10. In some aspects, the weight to weight ratio of the particles with diameters of about 1 μm to about 5 μm to the particles with sizes of about 7 μm to about 10 μm may range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10. In some aspects, the particles with larger sizes (about 10 μm to about 20 μm) may be mixed with particles with smaller sizes (about 1 μm to about 10 μm). In some aspects, the weight to weight ratio of the particles with larger sizes (about 10 μm to about 20 μm) to the particles with smaller sizes (about 1 μm to about 10 μm) may range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10. In some aspects, the weight to weight ratio of the particles with smaller sizes (about 1 μm to about 10 μm) to the particles with larger sizes (about 10 μm to about 20 μm) may range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10.

In some aspects, active ingredient particles may be produced by spray drying. In some cases, encapsulated active ingredient particles may be produce by spray drying. In some instances, active ingredient particles may be produced by another method. In some instances, active ingredient particles may be produced by air-jet micronization, spiral milling, controlled precipitation, high-pressure homogenization, or cryo-milling.

In some cases, microencapsulated particles herein can have one or more coatings (e.g. shells). For example, an active ingredient can comprise 1, 2, 3, 4, 5, 6, 7, 8, 9, or more than 9 coating layers. In some embodiments, the encapsulated active ingredient particles can be processed through a fluidized bed to apply an one or more additional coatings. For example, the encapsulated active ingredient particles can be processed through a fluidizer bed to apply an outer enteric coating.

In some aspects, particles that are not the pharmaceutically acceptable excipient, may have particle diameters ranging from about 1 μm to about 20 μm. In some aspects, particle diameters may be measured by a particle analyzer using laser diffraction (LD), static light scattering, dynamic light scattering (DLS), or nanoparticle tracking analysis (NTA).

In some aspects, active ingredient particles may comprise a hormone reception agonist drug such as a hormone receptor agonist. In some instances, a hormone receptor agonist may be blended with an excipient such as lactose or a salt thereof. In some instances, an excipient may comprise a lactose, a microcrystalline cellulose, a cellulose, a mannitol, a sorbitol, a starch, a starch glycolate, a hydroxypropyl methylcellulose, a hydroxypropyl methylcellulose acetate succinate, a cyclodextrin, a maltodextrin, a croscarmellose sodium, a corn starch, a carrageenan, a sorbitol, a maltitol, a glucose, a pharmaceutically acceptable salt of any of these, or any combination thereof.

In some cases, an encapsulated or unencapsulated hormone receptor agonist, or a salt thereof may have a mean particle diameter of more than, less than, or equal to about: 30 nm, 50 nm, 60 nm, 70 nm, 80 nm, 90 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, or 20 μm. In some cases, an excipient or a salt thereof may have a mean particle diameter of more than, less than, or equal to about: 30 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm 140 μm, 145 μm 150 μm, 155 μm 160 μm, 165 μm 170 μm, 175 μm 180 μm, 185 μm 190 μm, 195 μm, 200 μm, 205 μm, 210 μm, 215 μm 220 μm, 225 μm 230 μm, 235 μm 240 μm, 245 μm, or 250 μm. In some instances, the shell of the microencapsulated particle comprises HPMCAS or HPMC. In some cases, the shell of the microencapsulated particle may comprise trehalose. In some cases, microencapsulation a hormone receptor agonist by HPMCAS may provide faster absorption in the lungs. For example, a hormone receptor agonist may not be water soluble and microencapsulation with HPMCAS may provide increased absorption into the blood stream from the lungs. In some instances, microencapsulation may increase the solubility of an active ingredient. In some cases, a microencapsulated hormone receptor agonist may be absorbed about: 10% to about 70%, 5% to about 10%, 5% to about 20%, 10% to about 30%, 15% to about 40%, 25% to about 40%, 35% to about 50%, 10% to about 60%, 40% to about 90%, or 20% to about 50% faster than a hormone receptor agonist that is not microencapsulated. In some cases, a microencapsulated hormone receptor agonist, may be absorbed after inhalation into the blood stream in about: 5 seconds to about 30 seconds, 5 seconds to about 20 seconds, 10 seconds to about 20 seconds, 10 seconds to about 30 seconds, 10 seconds to about 60 seconds, 20 seconds to about 40 seconds, 30 second to about 60 seconds, 30 seconds to about 2 minutes, 1 minute to about 2 minutes or 1 minute to 5 minutes.

In some instances, a hormone receptor agonist, or a salt thereof, may be mixed with an excipient prior to adding to a capsule. In some cases, the mixing may comprise blending in a blender such as a V-type blender. In some cases, a hormone receptor agonist may be mixed in a V-type blender with an excipient. A V-type blender may include a Patterson Kelly/PK Blender, a Gemco or a Ross blender. In some instances, blending may be high shear or low shear blending. In some cases, blending may be high speed or low speed blending. In some cases, the blending may distribute the hormone agonist, or a salt thereof, and the excipient evenly. In some cases, the weight to weight ratio of the hormone agonist, or a salt thereof, and the excipient may be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 55:1, 60:1, 65:1, 70:1, 75:1, 80:1, 85:1, 90:1, 95:1, or 100:1 (hormone agonist to excipient). In some cases, the weight to weight ratio of the hormone agonist, or a salt thereof, and the excipient may be about: 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:55, 1:60, 1:65, 1:70, 1:75, 1:80, 1:85, 1:90, 1:95, or 1:100 (hormone agonist to excipient). In some aspects, an active ingredient or a pharmaceutically acceptable salt thereof (e.g., testosterone or a pharmaceutically acceptable salt thereof) may comprise at least about: 1%, 2%, 3%, 4%, 5%, 6%, 7%, 8%, 9%, or 10% by weight of a pharmaceutical composition. The blending may not cause the excipient particle to be coated by the hormone agonist particle or the salt thereof. In some instances, the hormone receptor agonist such as testosterone or a pharmaceutically acceptable salt thereof, and an excipient may be administered via inhalation by a dry powder inhaler. In some cases, a dry powder inhaler does not comprise a propellent. In some cases, a dry powder inhaler does not comprise a chlorofluorocarbon, a hydrofluorocarbon, a fluorocarbon or any combination thereof as a propellent. In some cases, a dry powder inhaler is not pressurized. In some cases, an inhaler can comprise a propellent. In some instances, inhalation administration of hormone agonist, or a salt thereof, and an excipient may produce about: 90%, 91%, 92%, 93%, 94%, 95%, 96%, 97%, 98%, 99% or 100% bioavailability of the hormone receptor agonist. In some cases, the Tmax (e.g., the time required to reach the maximum concentration of a drug in the plasma) may be about: 5 min, 10 min, 15 min, 20 min, 25 min, 30 min, 35 min, 40 min, 45 min, 50 min, 55 min, 60 min, 65 min, 70 min, 75 min, 80 min, 85 min, 90 min, 95 min, 100 min, 105 min, 110 min, 115 min, 120 min, 130 min, 140 min, 150 min, 160 min, 170 min, 180 min, 190 min, 200 min, 210 min, 220 min, 230 min, 240 min, 250 min, 260 min, 270 min, 280 min, 290 min, or 300 min for a hormone receptor agonist. In some cases, a composition can comprise a hormone receptor agonist in an amount of more than, less than, or equal to about: 10 μg, 25 μg, 50 μg, 75 μg, 100 μg, 150 μg, 200 μg, 220 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg.

In some aspects, a method of making a powdery pharmaceutical composition, may comprise blending: i) particles of a pharmaceutically acceptable excipient; and ii) a plurality of spray dried particles. In some cases, each particle of the plurality of spray dried particles may comprise a hormone receptor agonist such as testosterone or a pharmaceutically acceptable salt thereof. In some cases, the hormone receptor agonist can be substantially encapsulated in a coating material. In some cases, a portion of the plurality of spray dried particles comprising the hormone receptor agonist or the pharmaceutically acceptable salt thereof substantially encapsulated in the coating material may have a particle diameter ranging from about 1 micrometer to about 10 micrometers, or from about 1 micrometer to about 5 micrometers, as measured by a particle analyzer using laser diffraction. In some instances, the coating material may comprise trehalose, a hydroxypropyl methylcellulose (HPMC), a hydroxypropyl methylcellulose acetate succinate (HPMCAS), a cyclodextrin, a maltodextrin, a povidone, a copovidone or any combination thereof.

In some aspects, a powdery pharmaceutical composition may be produced by a process comprising: a) contacting the particles comprising a hormone receptor agonist (e.g., testosterone or a pharmaceutically acceptable salt thereof, a coating material, and a solvent and b) spray drying the mixed particles comprising the hormone receptor agonist, the coating material, and the solvent. In some cases, the spray dried particles may be mixed or blended with a pharmaceutically acceptable excipient to make a powdery pharmaceutical composition.

### Packaging of the Powdery Pharmaceutical Compositions

In some aspects, the pharmaceutical composition may be contained within a capsule, a capsule in capsule, a tablet, a gel, a gummy, a spray, an ointment, a paste, a jelly, an oil, a tincture, a lotion, a cream, a balm, a food, a drink, a liquid, a syrup, or any combination thereof. In some cases, a gummy can comprise a corn syrup, a sucrose, a glucose syrup, a sugar, a gelatin, a starch, a water, a sucralose, a fructose, a glucose, an allulose, a maltose, a sugar alcohol, an aspartame, a xylitol, a stevia, a galactose, a saccharin, a pectin, a carrageenan, a flavoring (e.g., a berry flavoring, a natural flavoring, an apple flavoring, a cherry flavoring, a watermelon flavoring, a strawberry flavoring) or any combination thereof. In some cases, a gummy can comprise an ethylene glycol, a glycerol, an erythritol, a threitol, an arabitol, a xylitol, a ribitol, a mannitol, a sorbitol, a galactitol, a fucitol, an iditol, an inositol, a volemitol, an isomalt, a maltitol, a lactitol, a maltotriitol, a maltotetraitol, a polyglycitol, or any combination thereof. In some cases, a gummy can comprise a pectin. In some cases, a gummy can comprise a gelatin. In some cases, a gummy can comprise a carrageenan. In some cases, the at least partially encapsulated particles can be substantially homogenous throughout the gummy.

In some aspects, the capsule may comprise a single-piece capsule, a two-piece capsule, a transparent capsule, a non-transparent capsule, an opaque capsule, a slow-release capsule, an extended-release capsule, a standard-release capsule, a rapid-release capsule, a quick-release capsule, a hard-shell capsule, a soft gel capsule, a gel capsule, a hard gelatin capsule, a soft gelatin capsule, an animal-based capsule, a vegetarian capsule, a polysaccharide capsule, a cellulose capsule, a mucopolysaccharide capsule, a tapioca capsule, a hydroxypropylmethyl cellulose (HPMC) capsule, a pullulan capsule, an enteric capsule, an uncoated capsule, a coated capsule, a capsule comprising titanium dioxide, fatty acids, waxes, shellac, plastics, plasticizers, glycerin, sorbitol, plant fibers, additives, preservatives, colorants, or any combination thereof.

In some aspects, the capsule can be sized according to pharmaceutical composition requirements. In some aspects, the capsule size is: 000, 00, 0, 1, 2, 3, or 4. In some aspects, the capsule size may be 000. In some aspects, the capsule size may be 00. In some aspects, the capsule size may be 0. In some aspects, the capsule size may be 1. In some aspects, the capsule size may be 2. In some aspects, the capsule size may be 3. In some aspects, the capsule size may be 4. In some aspects, the capsule capacity varies from about 0.21 ml to about 1.37 ml.

In some embodiments a capsule band can be added to a capsule. In some cases, capsule banding can be the process of sealing the capsule so that it may be filled with liquids, powders or other types of ingredients. In some instances, there can be a seal joint between the capsule cap and body that can require an additional band to be applied to prevent leakage of the drug outside the capsule. In some instances, it can provide a tamper resistant band that can reduce oxidation and minimizes any odor. In some cases, the banding can be applied with a banding machine that applies a thin layer of HPMC (hydroxypropyl methylcellulose) as the capsules pass over two rollers which apply the capsule banding material. In this process, the banding material can be heated and temperature controlled to make a smooth, liquid-tight band that join the capsule top and body. This can provide a visual tamper resistant barrier on the capsule.

In some aspects, the powdery pharmaceutical composition described herein when stored in a sealed container placed in a room at about: 20° C., 25° C., or 30° C. and a room atmosphere having about: 40 percent, 45 percent, 50 percent, 55 percent, or 60 percent relative humidity, retains at least about: 80%, 90%, 95%, 96%, 97%, 98%, or 99% of the active ingredient or the salt thereof after 4 month, 6 months, 12 months, or 24 months as measured by HPLC.

In some aspects, the pharmaceutical composition may be contained within a capsule. The capsule may be loaded with about 25% to about 75% (by volume) with the powdery pharmaceutical composition. In some cases, the capsule may be loaded with more than, less than or equal to about: 30%, 31%, 32%, 33%, 34%, 35%, 36%, 37%, 38%, 39%, or 40% (by volume) of a pharmaceutical composition described herein. In some aspects, the capsule may be loaded with about 25% to about 30%, about 25% to about 40%, about 25% to about 50%, about 25% to about 60%, about 25% to about 65%, about 25% to about 70%, about 25% to about 75%, about 30% to about 40%, about 30% to about 50%, about 30% to about 60%, about 30% to about 65%, about 30% to about 70%, about 30% to about 75%, about 40% to about 50%, about 40% to about 60%, about 40% to about 65%, about 40% to about 70%, about 40% to about 75%, about 50% to about 60%, about 50% to about 65%, about 50% to about 70%, about 50% to about 75%, about 60% to about 65%, about 60% to about 70%, about 60% to about 75%, about 65% to about 70%, about 65% to about 75%, or 70% to about 75%, (by volume) of the powdery pharmaceutical composition.

In some aspects, the content of the capsule comprises less than about: 10%, 9%, 8%, 7%, 6%, 5%, 4%, 3%, 2%, or 1% water by weight. In some aspects, the content of the capsule comprises less than about 50%, about 40%, about 30%, about 25%, about 20%, about 10%, about 5%, or 1% water by weight.

In some aspects, the total content of all gases in the capsule may be less than about: 10%, 9%, 8%, 7%, 6%, 5%, 4%, 3%, 2%, or 1% water by weight. In some aspects, the total content of all gases in the capsule may be less than about 50%, about 40%, about 30%, about 25%, about 20%, about 10%, about 5%, or 1% water by weight.

In some aspects, the capsule further comprises, in the volume not occupied by the powdery pharmaceutical composition, an inert gas. In some aspects, the inert gas comprises an elemental gas, a compound gas, a noble gas, helium, neon, argon, krypton, xenon, compounds of noble gas, purified argon, purified nitrogen, nitrogen, sulfur hexafluoride, or any combination thereof. In some aspects, the inert gas comprises nitrogen. In some cases, the inert gas within a capsule may comprise at least about: 75%, 80%, 85%, 90%, or 95% of the gas on a volume-to-volume basis.

In some aspects, the pharmaceutical composition may be contained within a capsule, wherein the capsule may be at least in part contained within a device which may be a drug delivery device, an inhalation drug delivery device, a diffuser, an inhaler, a metered dose inhaler, a dry powder inhaler, a soft mist inhaler, or any combination thereof. In some aspects, the device may be an inhaler. In some cases, a dry powder inhaler does not comprise a propellent. In some cases, a dry powder inhaler may not be pressurized. In some instances, the method of using a dry powder inhaler comprises breathing or inhaling an active ingredient or composition into the lungs. In some instances, a dry powder inhaler may be breath-activated, wherein when a subject breathes in through an inhaler, the inhaler releases particles (e.g., an active ingredient, excipient or both) which travel throughout the respiratory system. In some cases, a capsule may contain an active ingredient which may be pierced to release the particles prior to inhalation through a dry powder inhaler. In some instances, particle size and aerodynamics may affect travel throughout the respiratory system.

In some aspects, the pharmaceutical composition may be contained within a capsule, wherein the capsule may be at least in part contained within the device. In some aspects, the pharmaceutical composition may be contained within a capsule, wherein the capsule may be at least in part contained within the device, and wherein the device contains a sharp surface configured to puncture or slice the capsule. In some aspects, the pharmaceutical composition may be contained within a capsule, wherein the capsule may be at least in part contained within the device, and wherein the device contains a sharp surface configured to puncture or slice the capsule, and wherein, prior to administrating, the device may be actuated such that the sharp surface punctures or slices the capsule.

In some aspects, the pharmaceutical composition may be contained within a capsule, wherein the capsule may be at least in part contained within an inhaler, and wherein the inhaler contains a sharp surface configured to puncture or slice the capsule. In some aspects, the pharmaceutical composition may be contained within a capsule, wherein the capsule may be at least in part contained within an inhaler, and wherein the inhaler contains a sharp surface configured to puncture or slice the capsule, and wherein, prior to administrating, the inhaler may be actuated such that the sharp surface punctures or slices the capsule. In some aspects, the inhaler unit may be re-used via a process comprising replacing a spent capsule with a new capsule containing the powdery pharmaceutical composition. In some aspects, a component of the inhaler unit configured to at least in part hold the capsule may be temporarily at least partially separable from the inhaler unit. In some aspects, the capsule may be at least partially visible via an at least partially transparent material present in the inhaler unit.

Also disclosed herein are kits comprising the pharmaceutical composition contained at least in part in a packaging or a container. Also disclosed herein are methods of making kits comprising a pharmaceutical composition contained at least in part in a packaging or a container. In some cases, a container can comprise a plastic container, a metal container, a wood container, a glass container, or a combination thereof.

In some instances, the composition may be packaged to have a nominal load of about 3 mg to 30 mg. Based on the aerosol performance properties and concentration of the active agent in the dry powder composition, the composition may be packaged to have a delivered dose of at least about 0.1 mg to about 20 mg, at least about 0.25 mg to about 20 mg, at least about 0.5 mg to about 10 mg, at least about 0.25 mg to about 5 mg, or at least about 0.25 mg to about 3 mg. In some cases, the composition may be packaged to have a delivered dose of at least about 0.1 mg, about 0.25 mg, about 0.5 mg, about 1 mg, about 5 mg, about 10 mg, about 15 mg, or about 20 mg. In some instances, the composition may be packaged to have a delivered dose of about 0.25 mg to 20 mg, including delivered doses in the range of about 0.25 mg to about 5 mg, about 0.25 mg to about 2 mg, about 0.25 mg to about 3 mg, about 0.25 mg to about 4 mg, about 1 mg to about 5 mg, about 2 mg to about 8 mg, about 2 mg to about 12 mg, and about 5 mg to about 15 mg. In some instances, the composition may be packaged to have a delivered dose in the range of about 0.25 mg to about 5 mg, about 0.25 mg to about 2 mg, about 0.25 mg to about 3 mg, about 0.25 mg to about 4 mg, about 1 mg to about 5 mg, about 2 mg to about 8 mg, about 2 mg to about 12 mg, or about 5 mg to about 15 mg.

### Delivery of the Pharmaceutical Composition

In some aspects, the administration of the pharmaceutical composition or the second therapeutic may be administered orally, intra nasally, intra ocular, anally, by injection, intravenously, intramuscularly, subcutaneously, intraperitoneally, transdermally, by inhalation, or any combination thereof.

In some aspects, the administration of the pharmaceutical composition may be by inhalation. In some aspects, inhalation may be oral inhalation, intra nasal administration, or any combination thereof. In some aspects, the powdery pharmaceutical composition may be inhaled into human lungs. In some cases, inhaled may be inhalation through the mouth, for example with a dry powdered inhaler. In some cases, at least a portion of the excipient may deposit on the oropharynx. In some aspects, the powdery pharmaceutical composition, when inhaled into the lungs, provides a time to peak plasma concentration (Tmax) of the active ingredient or the salt thereof. The time to peak plasma concentration (Tmax) of the active ingredient or the salt thereof may range from about 1 minute to about one hour. In some aspects, the time to peak plasma concentration (Tmax) of the active ingredient or the salt thereof may range from about 1 minute to about ten minutes.

In some aspects, administering may be by oral ingestion, topical application, or inhalation. In some aspects, administering may comprise oral ingestion and the oral ingestion may comprise oral ingestion of a food, a liquid, a gel, a capsule, or any combination thereof. In some aspects, administering may comprise topical application and the topical application may comprise topical application of a lotion, a tincture, a balm, a cream, an oil, a gel, a butter, a liquid, a spray, an ointment, a paste, a jelly, or any combination thereof. In some aspects, administering may comprise inhalation and the inhalation may comprise inhalation by a diffuser, an inhaler, a nebulizer, or any combination thereof. In some aspects, administering may comprise inhalation and the inhalation may comprise inhalation by a diffuser or inhaler. In some aspects, administering may comprise inhalation and the inhalation may comprise inhalation by a nebulizer. In some aspects, administering may be performed at least about: 1 time per day, 2 times per day, 3 times per day, 4 times per day, 5 times per day, 6 times per day or more than 6 times per day. In some cases, administering may be performed daily, weekly, monthly, or as needed. In some aspects, administering may be conducted one, twice, three, or four times per day. In some cases, administration may be provided by a subject (e.g., the patient), a health care provider, or both.

In some aspects, administering may be performed for about: 1 day to about 8 days, 1 week to about 5 weeks, 1 month to about 12 months, 1 year to about 3 years, 3 years to about 10 years, 10 years to about 50 years, 25 years to about 100 years, or 50 years to about 130 years. In some aspects, the composition may be administered as needed, or for: one day, two days, three days, four days, five days, six days, a week, two weeks, three weeks, a month, two months, three months, four months, five months, six months, seven months, eight months, nine months, ten months, eleven months, a year, or chronically.

### Compositions for the Treatment of Diseases

Also disclosed herein are methods of treating or preventing a disease comprising treating or preventing the disease or condition by administering a therapeutically effective amount of the powdery pharmaceutical composition. For example, disclosed herein are methods of treating or preventing a disease comprising treating or preventing the disease or condition by administering, via inhalation, a therapeutically effective amount of the powdery pharmaceutical composition. In certain instances, the powdery pharmaceutical formulation can comprise a glucagon-like peptide 1 receptor agonist (GLP-1), GLP-1-RA, gastric inhibitory peptide receptor agonist, incretin, liraglutide, glargine, glimepiride, semaglutide, sitagliptin, insulin, a pharmaceutically acceptable salt of these, a derivative of these, or a combination of these. In certain cases, the powdery pharmaceutical formulation may have a human growth hormone receptor agonist comprising a human growth hormone peptide, a salt, a derivative, or a combination of these. This agonist may be used alone or in a pharmaceutical formulation to treat a condition. In some cases, treatment or prevention may be for a deficiency of human growth hormone. In certain cases, the pharmaceutical formulation can comprise a steroid hormone receptor agonist or a pharmaceutically acceptable salt thereof and be used to treat a disease or condition. In certain instances the steroid hormone receptor agonists or their pharmaceutically acceptable salts thereof can be an estrogen, a testosterone, a progesterone, or any combination thereof.

A pharmaceutical formulation may be used for the treatment or prevention of diabetes such as type 1 diabetes, type 2 diabetes, type 3c diabetes (pancreatogenic diabetes), a prediabetes, a gestational diabetes, an obesity, or a combination thereof. In some cases, a composition herein can be used to treat an overweight, a coronary artery disease (CAD), a coronary heart disease (CHD), an ischemic heart disease (IHD), a myocardial ischemia, or any combination thereof. In some cases, a formulation herein can be used in an alcohol use disorder, an opioid use disorder, a nonalcoholic fatty liver disease, a non-alcoholic steatohepatitis, a cardiovascular disease, or any combination thereof.

In some embodiments, a powdery pharmaceutical formulation herein can be used to treat a dementia. In some cases, dementia can comprise an Alzheimer's disease, a vascular dementia, a Lewy body disease, a frontotemporal dementia, an alcohol related dementia, a young onset dementia, a mild cognitive impairment, or any combination thereof. In some embodiments, a powdery pharmaceutical formulation herein can be used to treat a Parkinson's disease. In some embodiments, a powdery pharmaceutical formulation herein can be used to emotional well-being and/or mood.

In certain cases, a composition herein, such as a human growth hormone receptor agonist may be used to increase calcium retention, promote muscle mass increase, promote lipolysis, promote internal organ growth, reduce liver uptake of glucose, promote gluconeogenesis in the liver, promote maintenance and function of pancreatic islets, stimulate the immune system, improve cognitive function, treat turner syndrome, treat chronic kidney failure, treat Prader-Willi syndrome, treat intrauterine growth restriction, treat severe idiopathic short stature, maintain muscle mass from wasting in subjects suffering from AIDS, promote anti-aging, promote lean body mass, promote the maintenance or increase in body density, treating multiple sclerosis, promote weight loss, treat fibromyalgia, treat heart failure, treat a Crohn's disease, treat ulcerative colitis, treat burns or any combination thereof. In some cases, obesity can be defined as being overweight if an individual's body mass index (BMI) is between 25.0 and less than 30. In some instances, an individual may have class 1 obesity if the individual's BMI is 30 to less than 35. In some instances, an individual may have class 2 obesity if the individual's BMI is 30 to less than 40. In some instances, an individual may have class 3 obesity, which may be considered severe obesity of the BMI is 40 or higher.

In certain cases, the pharmaceutical formulation, such as a steroid hormone receptor agonist or a pharmaceutically acceptable salt thereof, can be used to treat conditions characterized by low testosterone levels such as a depression, a mood change, an irritability, an obesity, a fatigue, an osteoporosis, an osteopenia, a muscle atrophy, a sarcopenia, a sleep apnea, a hypogonadism, an erectile dysfunction, a low libido, an underdeveloped muscle mass, an underdeveloped secondary sex characteristics, an underdeveloped body hear growth, an underdeveloped voice deepening, an underdeveloped tendon, an underdeveloped ligament, a lack of spermatogenesis, or any combination thereof. In some cases, a depression can comprise a major depression, a melancholia, a psychotic depression, a antenatal depression, a postnatal depression, a dysthymic disorder, a bipolar disorder, a cyclothymic disorder, a seasonal affective disorder, or any combination thereof. In some cases, a treatment herein can be used to treat low-testosterone, low-estrogen, and/or low-progesterone levels. In certain instances a pharmaceutical formulation herein can be used to treat conditions characterized by a low estrogen level such as a menopause (e.g., a symptom from menopause), a regulation of the female reproductive system, a lack of female secondary sex characteristics, a low vaginal lubrication, an underdeveloped uterine growth, an unwanted bone reabsorption, a low bone formation, an undesirably low level of body fat, a thinning vaginal wall, an infertility, a regulation of ovulation, a regulation of menstruation, an endometriosis, a depression, an obsessive compulsive disorder, an eating disorder, a low libido, which can be found in both men and women and the like, or any combination thereof. In some cases, a symptom from menopause can comprise a hot flash, flushing, a night sweat, a memory loss, tender breasts, a vasomotor symptom, an irritability, a mood disturbance, a sleep disturbance, a fatigue, a decreased libido, a dyspareunia, an atrophic vaginitis, or any combination thereof.

In certain instances, a pharmaceutical formulation herein can be used to treat conditions characterized by low progesterone levels such as an uterine cancer, a cervical cancer, an inability to prevent pregnancy, an inability to maintain a pregnancy, an unwanted preterm labor, an unwanted lactation during pregnancy, an insufficient breast development for breastfeeding, a skin aging, a libido regulation, a symptom of menopause, or any combination thereof. In some cases, a pharmaceutical composition herein can be used to treat a thyroid disorder, an osteoporosis, a weight gain or any combination thereof. In some cases, a thyroid disorder can comprise hyperthyroidism, a hypothyroidism, Hashimoto's Thyroiditis, a thyroid cancer, a thyroid disorder in a woman, postpartum thyroiditis or any combination thereof.

In some aspects, the subject may be a human, a man, a woman, an individual over 18 years of age, an individual under 18 years of age, or any combination thereof. In some aspects, a subject may be from about 1 day to about 10 months old, from about 9 months to about 24 months old, from about 1 year to about 8 years old, from about 5 years to about 25 years old, from about 20 years to about 50 years old, from about 40 years to about 80 years old, or from about 50 years to about 130 years old.

In some embodiments, a powdery pharmaceutical formulation herein, such as metformin can be used to treat a type 1 diabetes, a type 2 diabetes, a type 3c diabetes (pancreatogenic diabetes), a prediabetes, a gestational diabetes, a polycystic ovary syndrome, obesity, weight gain from an antipsychotic medicine, a metabolic syndrome, a liver disease, a cardiovascular disease, a renal disease, and any combination thereof. In some embodiments, a powdery pharmaceutical formulation herein can be used to treat a metabolic syndrome. In some cases, a powdery pharmaceutical composition can be used for life extension, for health span extension or both. In some cases, the powdery pharmaceutical composition can comprise particles of metformin that are at least partially encapsulated. In some cases, the particles of metformin that are at least partially encapsulated are added to a gummy.

In some aspects, prior to treating, a subject may have been diagnosed with a disease. In some aspects, a method may further comprise diagnosing a subject as having the disease. In some aspects, a diagnosing may comprise employing an in vitro diagnostic. In some aspects, the in vitro diagnostic may be a companion diagnostic.

In some aspects, a diagnosis may comprise a physical examination, a radiological image, a blood test, an antibody test, or any combination thereof. In some aspects, a diagnosis may comprise a radiological image and the radiological image may comprise: a computed tomography (CT) image, an X-Ray image, a magnetic resonance image (MRI), an ultrasound image, or any combination thereof.

In some aspects, a method may further comprise administering a second therapy to the subject. In some cases, a composition may comprise an additionally therapy described above. For example, an additional therapy may comprise a nonsteroidal anti-inflammatory drug and the nonsteroidal anti-inflammatory drug may comprise, aspirin, naproxen, ibuprofen, diclofenac, celecoxib, mefenamic acid, etoricoxib, indomethacin, a salt of any of these, or any combination thereof. In some aspects, a composition may comprise an excipient, a diluent, a carrier, or any combination thereof. In some aspects, other pain non-opioid pain relievers may be used such as acetaminophens. In other aspects, opioid pain relievers may be used such as hydrocodone, oxycodone, morphine, codeine, hydromorphone, oxymorphone, a salt of any of these, or any combination thereof. In still other aspects, a composition may comprise caffeine, theobromine, a salt of any of these, or any combination thereof.

In some aspects, the composition may be administered so that the active ingredient or the pharmaceutically acceptable salt thereof in the unit dose ranges from about: 500 μg (micrograms) to about 1000 mg, 10 μg to about 50 μg, 40 μg to about 90 μg, 80 μg to about 120 μg, 100 μg to about 150 μg, 140 μg to about 190 μg, 150 μg to about 220 μg, 200 μg to about 250 μg, 240 μg to about 300 μg, 290 μg to about 350 μg, 340 μg to about 410 μg, 400 μg to about 450 μg, 440 μg to about 500 μg, 500 μg to about 700 μg, 600 μg to about 900 μg, 800 μg to about 1 mg, 1 mg to about 5 mg, 1 mg to about 10 mg, 5 mg to about 15 mg, 12 mg to about 25 mg, 20 mg to about 50 mg, 40 mg to about 80 mg, 70 mg to about 100 mg, 90 mg to about 150 mg, 125 mg to about 250 mg, 200 mg to about 500 mg, 400 mg to about 750 mg, 700 mg to about 900 mg, or from about 850 mg to about 1000 mg. In some cases, the unit dose range may be more than, or equal to about: 10 μg, 25 μg, 50 μg, 75 μg, 100 μg, 150 μg, 200 μg, 220 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg. In some cases, the unit dose range may be less than about: 10 μg, 25 μg, 50 μg, 75 μg, 100 μg, 150 μg, 200 μg, 220 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg. In some cases, hormone agonist or a salt thereof may be administered in a unit dose form of about 0.22 mg. In some cases, hormone agonist or a salt thereof may be administered in a unit dose form of about 0.10, 0.20, 0.30, 0.40 or 0.50 mg.

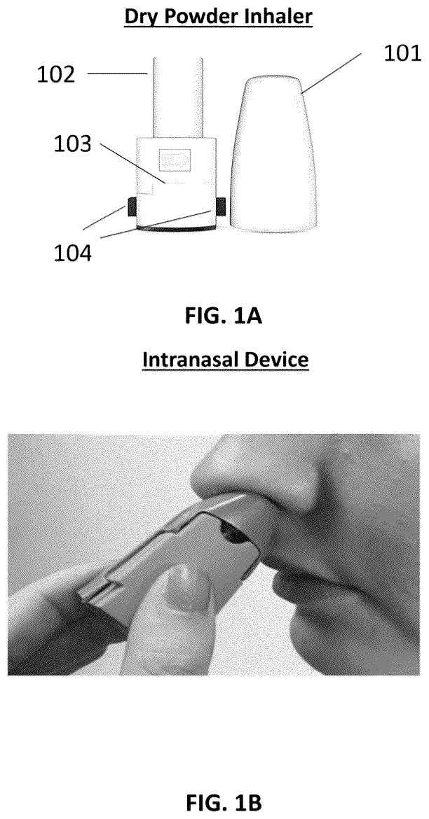

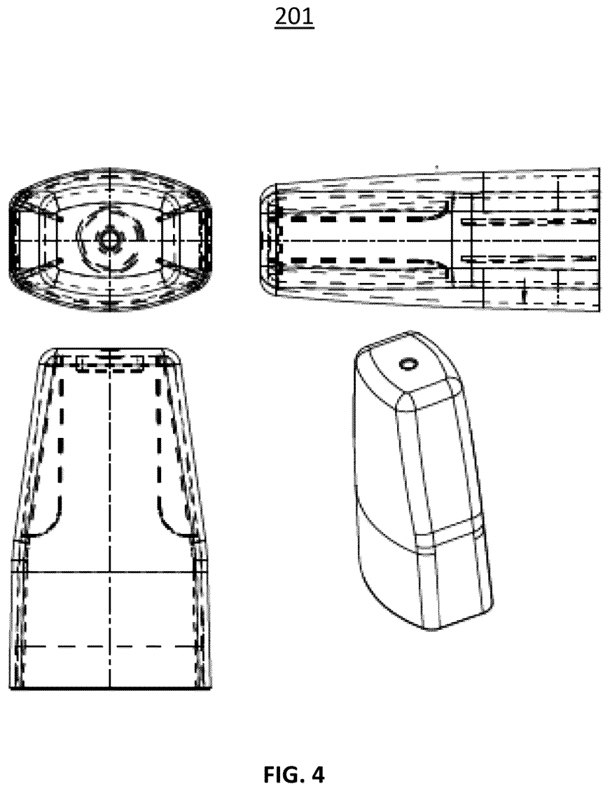

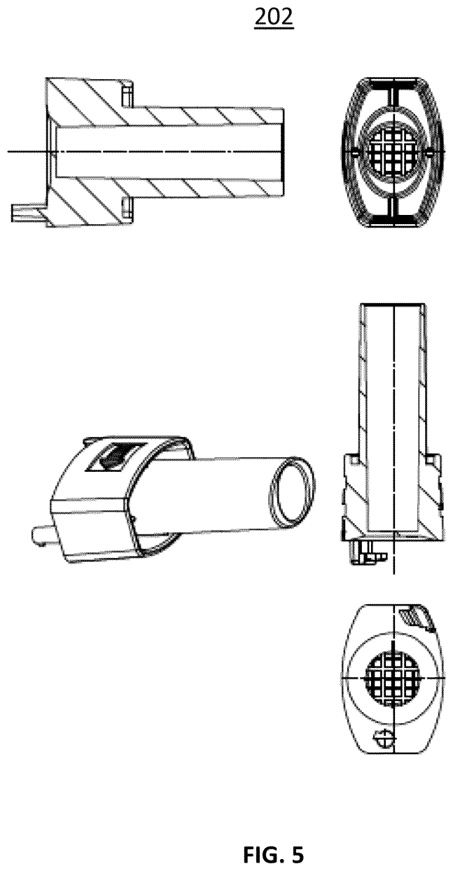

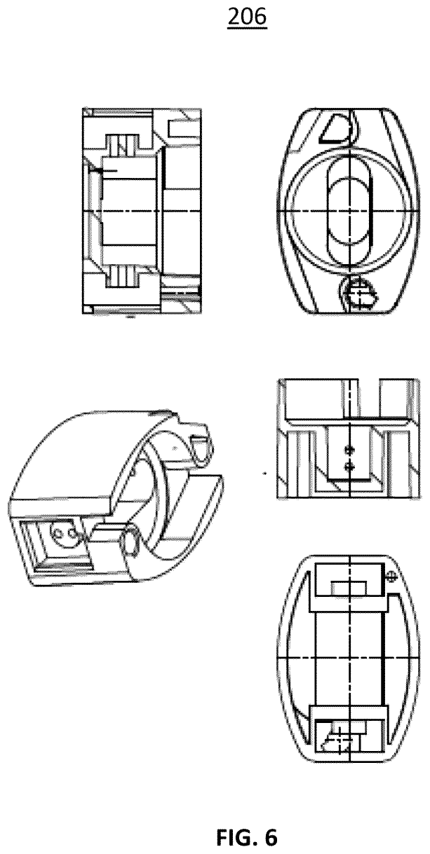

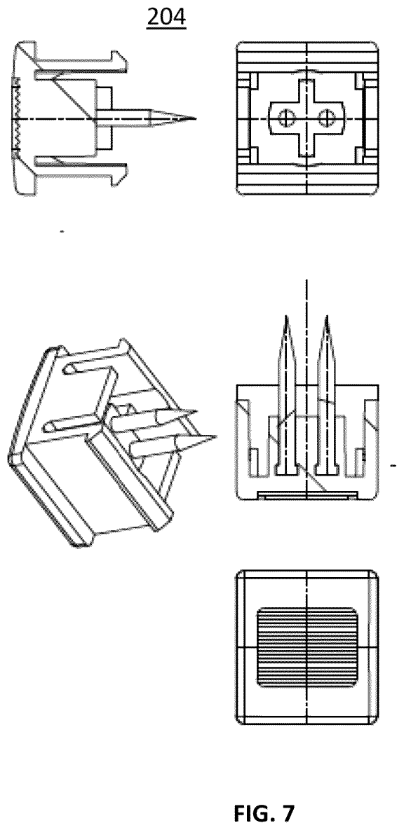

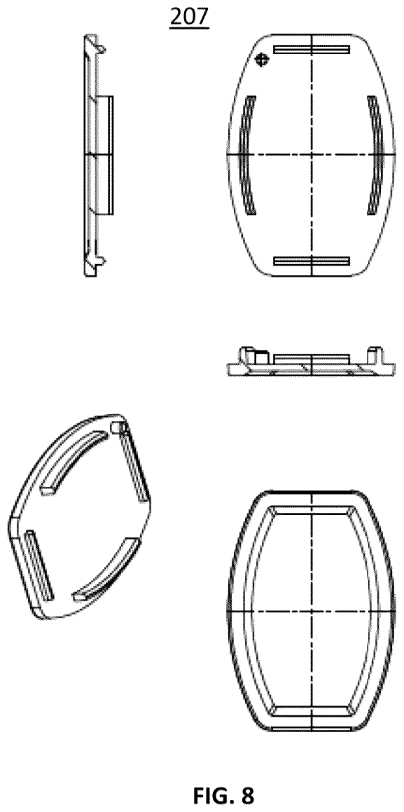

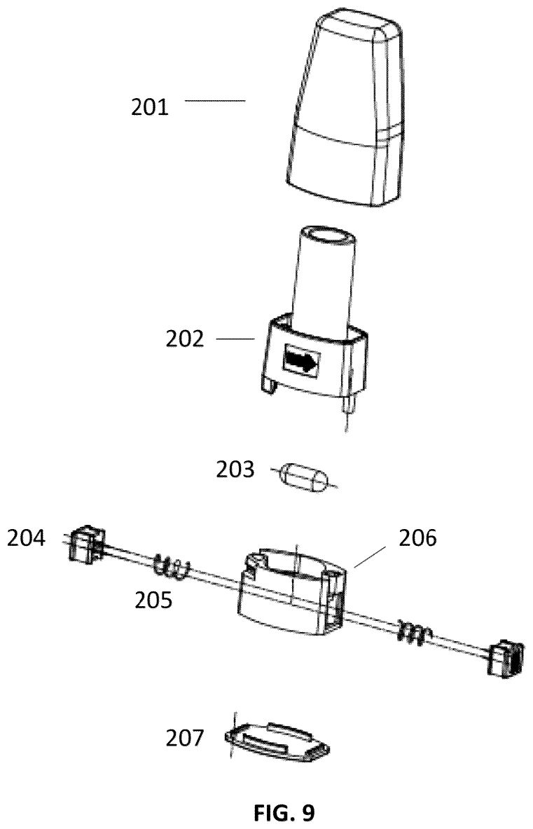

Referring to FIG. 1, FIG. 1A shows a dry powder inhaler device for delivery of powdery pharmaceutical compositions to the lung alveolar. The inhaler device may comprise a protective cap shown in FIG. 4, a rotatable top comprising a mouthpiece shown in FIG. 5, a lower base chamber receptacle for placing a pharmaceutical capsule shown in FIG. 6, a lateral button for mechanically piercing a capsule with a sharp surface while inside the chamber show in FIG. 7, and a chamber aerially connected to the mouthpiece permitting inhalation of capsule contents. The dry powder inhaler device may comprise a base plate as shown in FIG. 8. FIG. 9 shows a dry powder inhaler device with a protective cap, a rotatable comprising a mouthpiece, a lower base chamber for piercing a pill and a base plate.

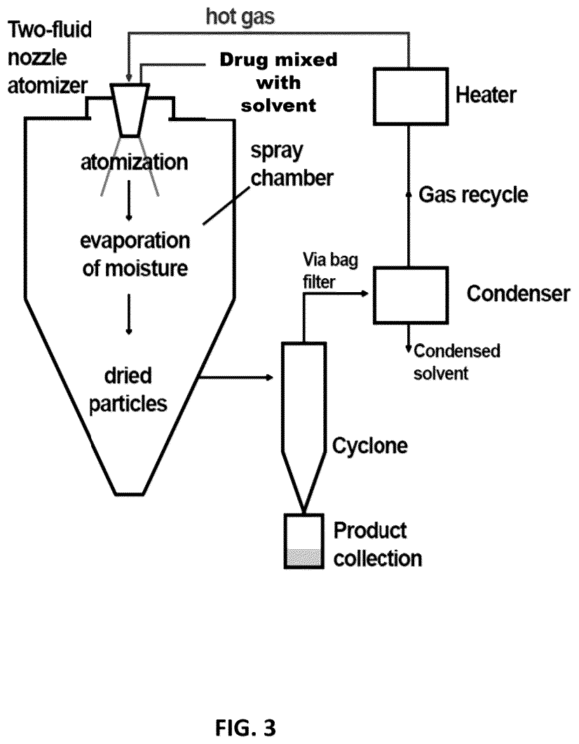

Referring to FIG. 3, FIG. 3 shows a spray drying manufacturing system comprising a closed spray dryer container which receives the solution comprising a drug dissolved or mixed in a suitable solvent (aqueous or solvent based). The solution then enters the particle formation chamber which is connected to an atomizer located at the top of the chamber. The atomizer is a two component or rotary nozzle type that distributes the solution into fine droplets controlled by the atomizer pressure. This atomization gas is an inert gas, either air or nitrogen. The atomized droplets go through a hot gas drying chamber to produce uniform fine particles that maintain a tight particle size distribution following liquid evaporation. Solid particle forms and falls to the bottom of the drying chamber. The balance between temperature, flow rate, and droplet size, controls the drying process. The powder is recovered from the exhaust gas using a cyclone or a bag filter. Particle size is validated by a Malvern particle analyzer prior to blending with an excipient carrier. The active powder is blended with an excipient carrier (lactose) product in a Patterson Kelly (PK Blender) and the blended powder is fed to a hopper. From the hopper, the dry powder is placed into a Size 3 Hypromellose capsule, by a Bosch Encapsulator machine.

In some aspects, a method of spray drying a liquid is disclosed herein. In some cases, a liquid may comprise i) a hormone agonist, or a pharmaceutically acceptable salt thereof; ii) a coating material, wherein the coating material may comprise trehalose, a hydroxypropyl methylcellulose (HPMC), a hydroxypropyl methylcellulose acetate succinate (HPMCAS), a cyclodextrin, a maltodextrin, a povidone, a copovidone or any combination thereof; and iii) a solvent, wherein the particles of the hormone agonist or the pharmaceutically acceptable salt thereof may be dispersed in the liquid. In some cases, the particles of the hormone agonist or the pharmaceutically acceptable salt thereof dispersed in the liquid may have a particle diameter ranging from about 1 micrometer to about 5 micrometers. In some instances, the spray drying may comprise i) atomizing liquid droplets comprising the hormone agonist or the pharmaceutically acceptable salt thereof, the coating material, and the solvent, ii) drying the droplets to form substantially encapsulated particles wherein the substantially encapsulated particles may comprise the hormone agonist or the pharmaceutically acceptable salt thereof substantially encapsulated by the coating material and iii) recovering the substantially encapsulated particles.

### NUMBERED EMBODIMENTS

A number of compositions, kits, and methods are disclosed herein. Specific exemplary embodiments of these compositions, kits, and methods are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1. A powdery pharmaceutical composition, comprising: a) particles of a pharmaceutically acceptable excipient; and b) a plurality of spray dried particles, wherein each particle of the plurality of spray dried particles is substantially encapsulated in a coating material and wherein the plurality of spray dried particles substantially encapsulated in the coating material comprise: an agonist selected from the group consisting of a steroid or a pharmaceutically acceptable salt thereof, a human growth hormone receptor agonist or a pharmaceutical acceptable salt thereof, and a glucagon-like peptide 1 receptor agonist or a pharmaceutical acceptable salt thereof; and wherein within the plurality of spray dried particles substantially encapsulated in the coating material at least a portion of the spray dried particles substantially encapsulated in the coating material individually have a particle diameter ranging from about 1 micrometer to about 10 micrometers, as measured by a particle analyzer using laser diffraction; and wherein the coating material comprises trehalose, a hydroxypropyl methylcellulose (HPMC), a fumaryl diketopiperazine (FDKP), 1,2-distearoyl-sn-glycero-3-phosphocholine, a hydroxypropyl methylcellulose acetate succinate (HPMCAS), a povidone, a copovidone, a lactose, a phospholipid or any combination thereof.

Embodiment 2. The powdery composition of embodiment 1, further comprising a cannabinoid or a pharmaceutically acceptable salt thereof comprising tetrahydrocannabinol Delta-8, tetrahydrocannabinol Delta-9, tetrahydrocannabinol Delta-10, tetrahydrocannabinol Delta-11, tetrahydrocannabinol Delta-13, tetrahydrocannabivarin (THCV), tetrahydrocannabinolic acid (THCA), full spectrum THC, broad spectrum THC, or a pharmaceutically acceptable salt thereof.

Embodiment 3. The powdery pharmaceutical composition of embodiment 1 or 2, wherein the spray dried particles comprises the steroid agonist or a pharmaceutically acceptable salt thereof.

Embodiment 4. The powdery pharmaceutical composition of embodiment 3, wherein the steroid agonist is testosterone or a pharmaceutically acceptable salt thereof.

Embodiment 5. The powdery pharmaceutical composition of embodiment 3, wherein the steroid agonist is an estrogen or a pharmaceutically acceptable salt thereof.

Embodiment 6. The powdery pharmaceutical composition of embodiment 3, wherein the steroid agonist is progesterone or a pharmaceutically acceptable salt thereof.

Embodiment 7. The powdery pharmaceutical composition of embodiment 1, wherein the spray dried particles comprise the human growth hormone receptor agonist or a pharmaceutically acceptable salt thereof.

Embodiment 8. The powdery pharmaceutical composition of embodiment 1, wherein the spray dried particles comprise the glucagon-like peptide 1 receptor agonist or a pharmaceutically acceptable salt thereof optionally, wherein the glucagon-like peptide 1 receptor agonist comprises a semaglutide, a liraglutide, or a pharmaceutically acceptable salt thereof.

Embodiment 9. The powdery pharmaceutical composition of any one of embodiments 1-8, wherein the powdery pharmaceutical composition is for inhaled use or for intranasal use.

Embodiment 10. The powdery pharmaceutical composition of any one of embodiments 1-9, wherein the pharmaceutical composition is in unit dose form.

Embodiment 11. The powdery pharmaceutical composition of any one of embodiments 1-10, wherein at least a portion of the particles of the pharmaceutically acceptable excipient individually have a particle diameter ranging from about 1 micrometers to about 200 micrometers, or about 1 micrometer to about 10 micrometers, as measured by a particle analyzer using laser diffraction.

Embodiment 12. The powdery pharmaceutical composition of any one of embodiments 1-11, wherein the particles are admixed into a substantially homologous mixture.

Embodiment 13. The powdery pharmaceutical composition of any one of embodiments 1-12, which is contained within a capsule.

Embodiment 14. The powdery pharmaceutical composition of embodiment 13, wherein the capsule is about one quarter to about one half, by volume, filled with the powdery pharmaceutical composition.

Embodiment 15. The powdery pharmaceutical composition of any one of embodiments 1-14, wherein a weight-to-weight ratio of: a) the particles of the pharmaceutically acceptable excipient and b) the particles comprising the agonist or a pharmaceutically acceptable salt thereof, substantially encapsulated in a coating material, ranges from about 1:1 (w/w) to about 10000:1 (w/w).

Embodiment 16. The powdery pharmaceutical composition of embodiment 15, wherein the weight-to-weight ratio of a) the particles of the pharmaceutically acceptable excipient and b) the particles of the agonist or the pharmaceutically acceptable salt thereof, ranges from about 1:1 (w/w) to about 10:1 (w/w).

Embodiment 17. The powdery pharmaceutical composition of any one of embodiments 13-16, wherein the portion of the capsule not containing the powdery pharmaceutical composition comprises a gas that at least partially comprises an inert gas.

Embodiment 18. The powdery pharmaceutical composition of embodiment 17, wherein the inert gas comprises nitrogen, carbon dioxide, helium, or any combination thereof.

Embodiment 19. The powdery pharmaceutical composition of embodiment 17 or 18, wherein the inert gas comprises at least about: 80%, 85%, 90%, or 95% of the gas on a volume-to-volume basis.

Embodiment 20. The powdery pharmaceutical composition of any one of embodiments 17-19, wherein the powdery pharmaceutical composition within the capsule, the gas within the capsule, or any combination thereof comprises less than about 10% water by weight based on the weight of the powdery pharmaceutical composition or a total content of all gases in the capsule is less than about 10% water by weight within: the powdery pharmaceutical composition within the capsule, the gas within the capsule, or any combination thereof.

Embodiment 21. The powdery pharmaceutical composition of any one of embodiments 13-20, wherein the capsule comprises a hydroxypropylmethyl cellulose (HPMC) capsule.

Embodiment 22. The powdery pharmaceutical composition of any one of embodiments 13-21, wherein the capsule is size: 000, 00, 0, 1, 2, 3, or 4.

Embodiment 23. The powdery pharmaceutical composition of embodiment 22, comprising the capsule, wherein the capsule is size 3.

Embodiment 24. The powdery pharmaceutical composition of any one of embodiments 1-23, contained within an inhaler unit.

Embodiment 25. The powdery pharmaceutical composition of any one of embodiments 13-24, wherein the capsule is contained in an inhaler unit.

Embodiment 26. The powdery pharmaceutical composition of any one of embodiments 1-25, wherein the pharmaceutically acceptable excipient comprises a carbohydrate, an alginate, povidone, a carbomer, a flavor, a natural gum, a silicone, an alcohol, a butter, a wax, a fatty acid, a preservative, a fumaryl diketopiperazine (FDKP), a pharmaceutically acceptable salt of any of these, or any combination thereof.

Embodiment 27. The powdery pharmaceutical composition of embodiment 26, wherein the pharmaceutically acceptable excipient or pharmaceutically acceptable salt thereof comprises the carbohydrate or the pharmaceutically acceptable salt thereof, and wherein the carbohydrate or the pharmaceutically acceptable salt thereof comprises lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, corn starch, carrageenan, sorbitol, maltitol, glucose, a pharmaceutically acceptable salt of any of these, or any combination thereof.

Embodiment 28. The powdery pharmaceutical composition of embodiment 27, wherein the pharmaceutically acceptable excipient or the pharmaceutically acceptable salt thereof comprises lactose or a pharmaceutically acceptable salt thereof.

Embodiment 29. The powdery pharmaceutical composition of embodiment 28, comprising the lactose or the pharmaceutically acceptable salt thereof, which comprises milled lactose, sieved lactose, micronized lactose, spray dried lactose, at least substantially anhydrous lactose, monohydrate lactose, a pharmaceutically acceptable salt thereof, or any combination thereof.

Embodiment 30. The powdery pharmaceutical composition of any one of embodiments 1-29, wherein the powdery pharmaceutical composition described herein when stored in a sealed container placed in a room at 25° C. and a room atmosphere having about 50 percent relative humidity, retains at least about: 90% of the agonist thereof after 6 months, as measured by HPLC.

Embodiment 31. The powdery pharmaceutical composition of any one of embodiments 1-30, wherein the agonist is present in an amount ranging from about 0.001 mg to about 20 mg.

Embodiment 32. The powdery pharmaceutical composition of any one of embodiments 1-31, wherein the agonist is in the form of a pharmaceutically acceptable salt thereof and is a hydrochloride salt, a bitartrate salt or a borate salt.

Embodiment 33. The powdery pharmaceutical composition of any one of embodiments 1-32, wherein the particles comprising the agonist comprise a median diameter of less than 5 μm.

Embodiment 34. The powdery pharmaceutical composition of any one of embodiments 1-33, wherein the particles comprising the agonist or the pharmaceutically acceptable salt thereof comprise a median diameter of less than about: 6 μm, 7 μm, 8 μm, 9 μm or 10 μm.

Embodiment 35. A kit comprising the powdery pharmaceutical composition of any one of embodiments 1-34 contained at least in part in a packaging.

Embodiment 36. A method of treating or preventing a disease or condition in a subject in need thereof, comprising treating or preventing the disease or condition by administering, via inhalation, a first therapeutic comprising a therapeutically effective amount of the powdery pharmaceutical composition of any one of embodiments 1-34 to the subject in need thereof.

Embodiment 37. The method of embodiment 36, wherein the administering is conducted one, twice, three, or four times per day.

Embodiment 38. The method of embodiment 36 or 37, wherein the agonist is the steroid agonist.

Embodiment 39. The method of embodiment 38, wherein the steroid agonist is testosterone.

Embodiment 40. The method of embodiment 39, wherein the disease or condition is selected from the group consisting of: obesity, low libido, underdeveloped muscle mass, underdeveloped secondary sex characteristics, depression, underdeveloped body hear growth, underdeveloped voice deepening, underdeveloped tendons and ligaments, and lack of spermatogenesis.

Embodiment 41. The method of embodiment 38, wherein the steroid agonist is an estrogen.

Embodiment 42. The method of embodiment 41, further comprising a cannabinoid or a pharmaceutically acceptable salt thereof comprising tetrahydrocannabinol (THC), cannabidiol (CBD), cannabinol (CBN), cannabigerol (CBG), cannabichromene (CBC), cannabicyclol (CBL), cannabivarin (CBV), cannabidivarin (CBDV), cannabichromevarin (CBCV), cannabigerovarin (CBGV), cannabigerol monomethyl ether (CBGM), and cannabidiolic acid (CBDA), cannabielsoin (CBE), cannabicitran (CBT), 10-Oxo-delta-6a-tetrahydrocannabinol (OTHC), cannabichromanon, cannabifuran, cannabiglendol, cannabiripsol, cannbicitran, or any combination thereof.

Embodiment 43. The method of embodiment 41 or 42, wherein the disease or condition is selected from the group consisting of: lack of female secondary sex characteristics, low vaginal lubrication, underdeveloped uterine growth, unwanted bone reabsorption, low bone formation, undesirably low levels of body fat, thinning vaginal walls, infertility, regulation of ovulation, regulation of menstruation, endometriosis, depression, obsessive compulsive disorder, and an eating disorder.

Embodiment 44. The method of embodiment 38, wherein the steroid agonist is progesterone.

Embodiment 45. The method of embodiment 44, further comprising a cannabinoid or a pharmaceutically acceptable salt thereof comprising tetrahydrocannabinol (THC), cannabidiol (CBD), cannabinol (CBN), cannabigerol (CBG), cannabichromene (CBC), cannabicyclol (CBL), cannabivarin (CBV), cannabidivarin (CBDV), cannabichromevarin (CBCV), cannabigerovarin (CBGV), cannabigerol monomethyl ether (CBGM), and cannabidiolic acid (CBDA), cannabielsoin (CBE), cannabicitran (CBT), 10-Oxo-delta-6a-tetrahydrocannabinol (OTHC), cannabichromanon, cannabifuran, cannabiglendol, cannabiripsol, cannbicitran, or any combination thereof.

Embodiment 46. The method of embodiment 44 or 45, wherein the disease or condition is selected from the list consisting of: uterine cancer, cervical cancer, inability to prevent pregnancy, inability to maintain a pregnancy, unwanted preterm labor, unwanted lactation during pregnancy, insufficient breast development for breastfeeding, skin aging, and libido regulation.

Embodiment 47. The method of embodiment 36 or 37, wherein the agonist is human growth hormone.

Embodiment 48. The method of embodiment 47, wherein the disease or condition is selected from the group consisting of: low calcium retention, low muscle mass, obesity, low internal organ growth, high liver uptake of glucose, low gluconeogenesis in the liver, dysfunction of pancreatic islets, poor immune system stimulation, low cognitive function, turner syndrome, chronic kidney failure, Prader-Willi syndrome, intrauterine growth restriction, severe idiopathic short stature, muscle mass wasting from AIDS, low body density, multiple sclerosis, obesity, fibromyalgia, heart failure, Chron's disease, and ulcerative colitis.

Embodiment 49. The method of embodiment 36 or 37, wherein the agonist is the GLP-1 receptor agonist.

Embodiment 50. The method of embodiment 49, wherein the disease or condition is selected from the list of: type 1 diabetes, type 2 diabetes, type 3c diabetes (pancreatogenic diabetes), obesity, or a combination thereof, obesity, overweight, coronary artery disease (CAD), also called coronary heart disease (CHD), ischemic heart disease (IHD), and myocardial ischemia.

Embodiment 51. The method of any one of embodiments 36-37, wherein the powdery pharmaceutical composition is administered as needed, or for about: one day, two days, three days, four days, five days, six days, a week, two weeks, three weeks, a month, two months, three months, four months, five months, six months, seven months, eight months, nine months, ten months, eleven months, a year, or chronically.

Embodiment 52. The method of any one of embodiments 36-51, wherein a second therapeutic or pharmaceutically acceptable salt thereof is administered.

Embodiment 53. The method of embodiment 52, wherein the second therapeutic or a pharmaceutically acceptable salt thereof is administered concurrently or consecutively.

Embodiment 54. The method of embodiment 52 or 53, wherein the second therapeutic or the pharmaceutically acceptable salt thereof is comprised in the powdery pharmaceutical formulation.

Embodiment 55. The method of embodiment 52, wherein when the first therapeutic comprises the steroid receptor agonist, the second therapeutic comprises the human growth hormone receptor agonist.

Embodiment 56. The method of any one of embodiments 36-55, wherein the subject is diagnosed with the disease or condition.

Embodiment 57. The method of embodiment 56, wherein the diagnosing comprises employing an in vitro diagnostic.

Embodiment 58. The method of embodiment 57, wherein the in vitro diagnostic is a companion diagnostic.

Embodiment 59. The method of any one of embodiments 36-58, wherein the powdery pharmaceutical composition is contained within a capsule, wherein the capsule is at least in part contained within an inhaler, and wherein the inhaler contains a sharp surface configured to puncture or slice the capsule, and wherein, prior to administrating, the inhaler is actuated such that the sharp surface punctures or slices the capsule.

Embodiment 60. The method of any one of embodiments 36-59, wherein the inhalation is oral inhalation, intra nasal administration, or any combination thereof.

Embodiment 61. A method for making a powdery pharmaceutical composition, comprising contacting in a solution: a) an agonist comprising an agonist selected from the group consisting of: a steroid or a pharmaceutically acceptable salt thereof, a human growth hormone receptor agonist or a pharmaceutical acceptable salt thereof, and a glucagon-like peptide 1 receptor agonist or a pharmaceutical acceptable salt thereof; b) a coating material comprising trehalose, a fumaryl diketopiperazine (FDKP), 1,2-distearoyl-sn-glycero-3-phosphocholine, a hydroxypropyl methylcellulose (HPMC), a hydroxypropyl methylcellulose acetate succinate (HPMCAS), a povidone, a copovidone, lactose, a phospholipid, or any combination thereof, and c) a solvent; wherein the solution is spray dried to form substantially encapsulated particles.

Embodiment 62. The powdery pharmaceutical composition of embodiment 61, wherein the spray drying comprises: a) atomizing liquid droplets comprising the agonist or the pharmaceutically acceptable salt thereof, the coating material, and the solvent, b) drying the droplets to form the substantially encapsulated particles, wherein the substantially encapsulated particles comprise the agonist or the pharmaceutically acceptable salt thereof substantially encapsulated by the coating material and b) recovering the substantially encapsulated particles.

Embodiment 63. A method of treating or preventing a disease or condition in a subject in need thereof, comprising treating or preventing the disease or condition by administering, via inhalation, a first therapeutic comprising a therapeutically effective amount of the powdery pharmaceutical composition of embodiment 1 to the subject in need thereof.

Embodiment 64. The method of embodiment 63, wherein the administering is conducted one, twice, three, or four times per day.

Embodiment 65. The method of embodiment 63 or 64, wherein the disease or condition is selected from the group consisting of: obesity, low muscle mass, or infertility.

Embodiment 66. A powdery pharmaceutical composition, comprising: particles of a pharmaceutically acceptable carrier; and a plurality of spray dried particles, wherein each particle of the plurality of spray dried particles is substantially unencapsulated and wherein the plurality of spray dried particles substantially unencapsulated comprise: an agonist selected from the group consisting of: a steroid or a pharmaceutically acceptable salt thereof, a human growth hormone receptor agonist or a pharmaceutical acceptable salt thereof, and a glucagon-like peptide 1 receptor agonist or a pharmaceutical acceptable salt thereof; and wherein within the plurality of spray dried particles have mass median aerodynamic diameter of less than 5 μm and a fine particle fraction of at least about 40% upon aerosolization; and wherein pharmaceutically acceptable carrier comprises trehalose, a hydroxypropyl methylcellulose (HPMC), a fumaryl diketopiperazine (FDKP), 1,2-distearoyl-sn-glycero-3-phosphocholine, a hydroxypropyl methylcellulose acetate succinate (HPMCAS), a povidone, a copovidone, a lactose, a phospholipid, or any combination thereof.

Embodiment 67. The pharmaceutical composition of embodiment 66, wherein the composition comprises at least about 2% to about 20% of the agonist.

Embodiment 68. The pharmaceutical composition of embodiment 67, wherein the agonist is the steroid agonist or a pharmaceutically acceptable salt thereof.

Embodiment 69. The pharmaceutical composition of embodiment 67, wherein the steroid agonist is testosterone or a pharmaceutically acceptable salt thereof.

Embodiment 70. The pharmaceutical composition of embodiment 67, wherein the steroid agonist is an estrogen or a pharmaceutically acceptable salt thereof.

Embodiment 71. The pharmaceutical composition of embodiment 67, wherein the steroid agonist is progesterone or a pharmaceutically acceptable salt thereof.

Embodiment 72. The pharmaceutical composition of embodiment 67, wherein the agonist comprises the human growth hormone receptor agonist or a pharmaceutically acceptable salt thereof.

Embodiment 73. The pharmaceutical composition of embodiment 67, wherein the agonist comprises the human growth hormone receptor agonist or a pharmaceutically acceptable salt thereof.

Embodiment 74. The pharmaceutically composition of embodiment 67, wherein the agonist comprises the glucagon-like peptide receptor agonist or a pharmaceutically acceptable salt thereof.

Embodiment 75. The pharmaceutical composition of any one of embodiment 66-74, wherein the pharmaceutical composition is packaged to have a nominal load of about 3 mg to about 30 mg.

Embodiment 76. The pharmaceutical composition of any one of embodiments 66-74, wherein the pharmaceutical composition is packaged to have a nominal dose of at least about 0.25 mg.

Embodiment 77. The pharmaceutical composition of any one of embodiments 66-74, wherein the composition is packaged to have a delivered dose of at least about 0.075 mg.

### Embodiments Section 2

Embodiment 1. A powdery composition, comprising: a plurality of spray dried particles, wherein each particle of the plurality of spray dried particles is at least partially encapsulated by a coating, and wherein the plurality of at least partially encapsulated spray dried particles comprise a metformin or a pharmaceutically acceptable salt thereof, wherein
within the plurality of the at least partially encapsulated spray dried particles, the plurality of at least partially encapsulated spray dried particles individually have a particle diameter ranging from about 20 micrometers to about 200 micrometers as measured by a particle analyzer using laser diffraction,
wherein the coating comprises a hydroxypropyl methylcellulose (HPMC), a hydroxypropyl methylcellulose acetate succinate (HPMCAS), a cyclodextrin, a maltodextrin, a povidone, a copovidone, a methyl methacrylate (MMA), an enteric coating or any combination thereof.

Embodiment 2. The powdery composition of embodiment 1, wherein the coating comprises a methyl methacrylate (MMA).

Embodiment 3. The powdery composition of embodiment 1, wherein the coating comprises an enteric coating.

Embodiment 4. The powdery composition of embodiment 1, wherein the metformin or the pharmaceutically acceptable salt thereof is present in an amount ranging from 100 mg to about 3000 mg or about 500 mg to about 2500 mg.

Embodiment 5. The powdery composition of embodiment 1, wherein the powdery composition is comprised in a gummy.

Embodiment 6. The powdery composition of embodiment 5, wherein the gummy comprises a gelatin, a pectin, a carrageenan or any combination thereof.

Embodiment 7. A kit comprising the powdery composition of any one of embodiments 1-6 and a container.

Embodiment 8. A method of treating a disease or condition in a subject in need thereof, comprising treating the disease or condition by orally administering a therapeutically effective amount of the powdery composition of any one of embodiments 1-6 to the subject in need thereof.

Embodiment 9. The method of embodiment 8, wherein the disease or condition is selected from the group consisting of: a type 1 diabetes, a type 2 diabetes, a type 3c diabetes (pancreatogenic diabetes), a prediabetes, a gestational diabetes, a polycystic ovary syndrome, obesity, weight gain from an antipsychotic medicine, a liver disease, a cardiovascular disease, a renal disease, and any combination thereof.

Embodiment 10. The method of embodiment 8 or 9, wherein the administering is conducted one, twice, three, or four times per day.

Embodiment 11. The method of any one of embodiments 8-10, wherein the powdery pharmaceutical composition is administered as needed, or for about: one day, two days, three days, four days, five days, six days, a week, two weeks, three weeks, a month, two months, three months, four months, five months, six months, seven months, eight months, nine months, ten months, eleven months, a year, or chronically.

Embodiment 12. The method of any one of embodiments 8-11, wherein a second therapeutic or a pharmaceutically acceptable sat thereof is administered, and optionally wherein the second therapeutic is administered concurrently or consecutively.

### EXAMPLES

The following examples are included for illustrative purposes only and are not intended to limit the scope of the disclosure.

### Example 1

The powdery pharmaceutical composition described herein is administered by a dry powder inhaler or by a nasal inhaled device. Referring to FIG. 1A, FIG. 1A shows a dry powder inhaler device for delivery of a powdery pharmaceutical composition described herein to the lung alveolar. The inhaler device comprises: a protective cap 101, a rotatable top comprising a mouthpiece 102, a lower base chamber receptacle for placing the pharmaceutical capsule 103, lateral buttons for mechanically piercing the capsule with a sharp surface while inside the chamber 104, wherein the chamber is aerially connected to the mouthpiece permitting inhalation of capsule contents. FIG. 1B shows the nasal administration by a nasal inhaled device of a powdery pharmaceutical composition in a human subject. The composition is inhaled via the nares after the capsule containing the composition is pierced within the nasal inhalable device.

### Example 2

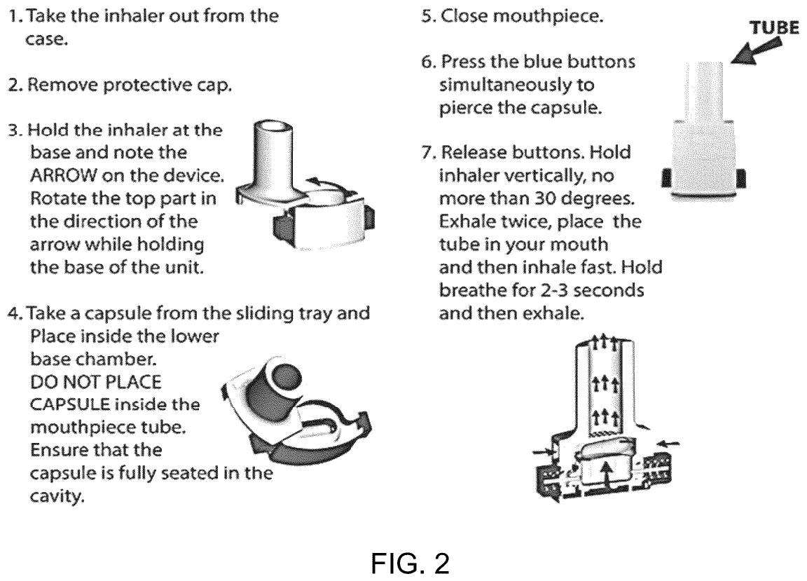

The method of using an inhaler device for the administration of a dry powdery pharmaceutical composition is shown in FIG. 2. The process for administration of the dry powdery pharmaceutical composition comprises 7 steps. Step 1: The inhaler is removed from the case. Step 2: The protective cap is removed. Step 3: The inhaler is held at the base and the top part is rotated in the direction of the arrow while the base of the unit is held. Step 4: A capsule is placed inside the lower base chamber cavity. Step 5: The mouthpiece is closed. Step 6: The buttons are pressed simultaneously to piece the capsule. Step 7: The buttons are released. The inhaler is held vertically, e.g., no more that about 30 degrees. The subject exhales twice before placing the tube in their mouth. The subject inhales quickly and holds their breath for about 2-3 seconds before exhaling.

### Example 3

The active ingredients (e.g., a hormone receptor agonist) in a dry powdery pharmaceutical composition described herein is manufactured by a spray drying system. FIG. 3 shows a spray drying manufacturing system comprising a closed spray dryer container which receives the solution comprising a drug dissolved or mixed in a suitable solvent (aqueous or solvent based). The solution then enters the particle formation chamber which is connected to an atomizer located at the top of the chamber. The atomizer is a two component or rotary nozzle type that distributes the solution into fine droplets controlled by the atomizer pressure. This atomization gas is an inert gas, such as air, nitrogen or carbon dioxide. The atomized droplets go through a hot gas drying chamber to produce uniform fine particles that maintain a tight particle size distribution following liquid evaporation. Solid particles form and fall to the bottom of the drying chamber. The balance between temperature, flow rate, and droplet size controls the drying process. The powder is recovered from the exhaust gas using a cyclone or a bag filter. Particle size is validated by a Malvern particle analyzer prior to blending with an excipient carrier. The active powder is blended with an excipient carrier (lactose) product in a Patterson Kelly (PK Blender) and the blended powder is fed to a hopper. From the hopper, the dry powder is placed into a Size 3 Hypromellose capsule, by a Bosch Encapsulator machine.

### Example 4

A male subject has low muscle mass. The subject is administered a pharmaceutical composition in combination with an exercise regimen weekly for six months. The pharmaceutical composition comprises testosterone which has been processed to a dry powder using the methods described herein (e.g., spay drying). The dry powder is mixed with a lactose powder and encapsulated. The hormone agonist is packaged in a capsule and is administered intranasally with an inhaler. The dosing regimen comprises an effective amount of testosterone increase muscle mass with an exercise regimen. The absorption of the inhaled pharmaceutical composition reaches the blood stream at least 5× faster than a comparable pharmaceutical composition that is administered orally.

### Example 5

The powdery pharmaceutical composition described herein is administered by a dry powder inhaler. Referring to FIG. 9, FIG. 9 shows a dry powder inhaler device for delivery of a powdery pharmaceutical composition described herein to the lung alveolar. The inhaler device comprises: a protective cap 201, a rotatable top comprising a mouthpiece 202, a lower base chamber receptacle 206 for placing the pharmaceutical capsule 203, lateral buttons for mechanically piercing the capsule with a sharp surface 204 while inside the chamber with the use of a spring 205, wherein the chamber is aerially connected to the mouthpiece permitting inhalation of capsule contents. The baseplate 207 is fitted to the lower base chamber receptacle.

### Example 6

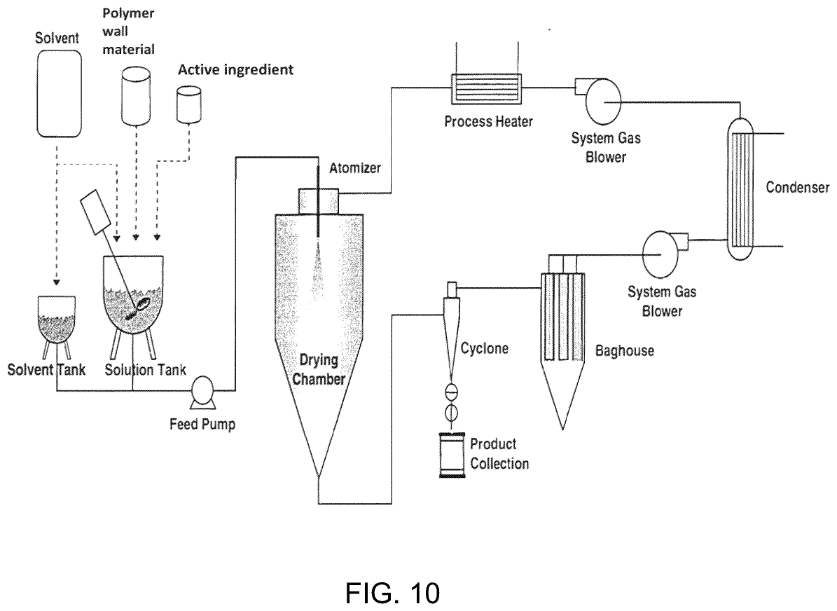

Testosterone in a dry powdered pharmaceutical composition was manufactured by a spray drying system. FIG. 10 shows a spray drying manufacturing system comprising a closed spray drying chamber which receives the solution comprising a polymer wall material (i.e., HPMCAS) dissolved in a solvent (e.g., 70% ethanol and 30% water) and an active ingredient (i.e., testosterone). The dissolved polymer wall material and testosterone were thoroughly mixed into a liquid suspension. The liquid suspension was then fed into an atomizer located at the top of the chamber. The atomizer is a two component or rotary nozzle type that distributes the solution into fine droplets controlled by the atomizer pressure. This atomization gas is an inert gas, such as air, nitrogen or carbon dioxide. The atomized droplets were then sent through a drying chamber with hot gas to produce uniform fine particles that maintain a tight particle size distribution following liquid evaporation. Solid particles were formed and fell to the bottom of the drying chamber as amorphous crystals. The balance between temperature, flow rate, and droplet size, were used to control the drying process. The powder was recovered from the exhaust gas using a cyclone or a bag filter. Particle size was validated by a Malvern particle analyzer prior to blending with an excipient carrier.

The active powder was blended with an excipient carrier (i.e., lactose) product in a Patterson Kelly (PK Blender) and the blended powder was fed to a hopper. From the hopper, the dry powder was placed into a Size 3 Hypromellose capsule, by a Bosch Encapsulator machine. Small amounts of microencapsulated testosterone was blended with a V-type blender using an intensifier bar that operated at high speeds to distribute the active powder uniformly into the excipient carrier. The V-Blenders are manufactured by Patterson Kelly/PK Blender, Gemco or Ross blenders.

The blended powder was then loaded into the hopper of the encapsulator machine (“encapsulator”), which fed the powder into the capsules. The encapsulator automatically separated the capsule top (“cap”) and body (“shell”) and the powder was slugged and then transferred into the body of the capsule. The capsule halves were closed together to form an enclosed capsule that contained the blended powder. During filling, the capsule atmosphere was made inert with nitrogen to prevent oxidation and remove moisture from the blend so that the inhalable powder was able flow freely from the capsule using the dry powder inhaler. The dry powder was placed into a Hypromellose capsule, by a Bosch, ACG or IMA Encapsulator machine.

### Example 7

A male subject has a low libido. The subject is administered a pharmaceutical composition to treat the low libido. The pharmaceutical composition comprises encapsulated hormone agonist (e.g., testosterone) which is processed to a dry powder using the methods described herein (e.g., spay drying). The dry powder is mixed with a lactose powder and encapsulated. The hormone agonist is packaged in a capsule and is administered intranasally with an inhaler. The dosing regimen comprises an effective amount of hormone agonist to treat the low libido.

### Example 8

A male subject is diagnosed with an obesity. The subject is administered a pharmaceutical composition to treat the obesity. The pharmaceutical composition comprises encapsulated liraglutide which is processed to a dry powder using the methods described herein (e.g., spay drying). The dry powder is mixed with a lactose powder and encapsulated. The liraglutide is packaged in a capsule and is administered with a dry powdered inhaler. The dosing regimen comprises an effective amount of liraglutide to induce weight loss as measured by measuring the subject's weight at the start of a treatment regimen and comparing to the subject's weight after the treatment regimen. Administration continues daily, or weekly until a desired weight loss is achieved.

### Example 9

A crystalized testosterone containing ˜99% Testosterone is used to prepare a testosterone inhalation powder by spray drying. Testosterone (3.9 g) is dissolved in approximately 290 ml of 200 proof ethanol. A separate solution of 1,2-distearoyl-sn-glycero-3-phosphocholine (1.5 g) in approximately 50 ml of 200 proof ethanol is prepared, then added to the testosterone solution. FDKP (9.6 g), approximately 50 ml of ethanol, and 98 ml of water is added to the testosterone mixture. The resulting suspension is spray dried in a system equipped with an inert loop, a dehumidifier, and a chiller. The spray drier is operated at 10% feed rate, 90% aspirator rate, 60 mm nitrogen flow rate, and an inlet temperature of 150° C. The resulting powder is analyzed by high performance liquid chromatography to determine percent drug content.

### Example 10

A subject is diagnosed with an type 2 diabetes. The subject is administered a pharmaceutical composition to treat the type 2 diabetes. The pharmaceutical composition comprises microencapsulated metformin which is processed to an encapsulated dry powder using the methods described herein (e.g., spay drying). The particles of metformin are microencapsulated with an enteric coating, for example methyl methacrylate (MMA). The microencapsulated metformin powder is mixed with a gelatin and other ingredients to form a gummy. The metformin gummy is administered to the subject. The dosing regimen comprises an effective amount of metformin to treat the type 2 diabetes. Administration continues daily to the treat the type 2 diabetes.

### Example 11

Seven male subjects were administered microencapsulated testosterone to replace testosterone injections. The pharmaceutical composition comprised HPMCAS microencapsulated testosterone, which was processed to a dry powder using the methods described herein. The dry powder was mixed with an excipient. The encapsulated testosterone and excipient were packaged in a capsule and were administered orally with an inhaler. Four of the seven subjects showed a substantial increase in testosterone levels after testing with an in vitro test to measure testosterone levels.

### Example 12

A subject was administered microencapsulated liraglutide for appetite suppression. The pharmaceutical composition comprised HPMCAS microencapsulated liraglutide, which was processed to a dry powder using the methods described herein. The dry powder was mixed with an excipient. The encapsulated liraglutide and excipient were packaged in a capsule and were administered orally with an inhaler. The subject noted no side effects from the administration of liraglutide.

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the methods presented in the disclosure. It is intended that the following claims define the scope of the disclosure and that methods and structures within the scope of these claims and their equivalents be covered thereby.

What is claimed is:

1. A pharmaceutical composition in unit dose form, comprising:
a plurality of spray dried particles, wherein each particle of the plurality of spray dried particles is substantially encapsulated in a coating material and wherein the plurality of spray dried particles substantially encapsulated in the coating material comprise a glucagon-like peptide 1 receptor agonist or a pharmaceutically acceptable salt thereof;
wherein at least a portion of the plurality of spray dried particles substantially encapsulated in the coating material individually have a particle diameter ranging from about 10 micrometers to about 200 micrometers, as measured by a particle analyzer using laser diffraction; and
the coating material comprises a hydroxypropyl methylcellulose acetate succinate (HPMCAS).

2. The pharmaceutical composition in unit dose form of claim 1, comprising the glucagon-like peptide 1 receptor agonist.

3. The pharmaceutical composition in unit dose form of claim 2, wherein the glucagon-like peptide 1 receptor agonist is semaglutide.

4. The pharmaceutical composition in unit dose form of claim 3, wherein the semaglutide is present in the composition in an amount of about 14 mg.

5. The pharmaceutical composition in unit dose form of claim 4, that is contained in a capsule.

6. The pharmaceutical composition in unit dose form of claim 3, further comprising a phospholipid.

7. The pharmaceutical composition in unit dose form of claim 4, further comprising a phospholipid.

8. The pharmaceutical composition in unit dose form of claim 6, wherein the phospholipid comprises a phosphatidylcholine (PC).

9. The pharmaceutical composition in unit dose form of claim 7, wherein the phospholipid comprises a phosphatidylcholine (PC).

10. The pharmaceutical composition in unit dose form of claim 8, wherein a weight to weight ratio of the semaglutide to the phosphatidylcholine (PC) is about 1:3.

11. The pharmaceutical composition in unit dose form of claim 9, wherein a weight to weight ratio of the semaglutide to the phosphatidylcholine (PC) is about 1:3.

12. The pharmaceutical composition in unit dose form of claim 10, that is comprised in a capsule.

13. The pharmaceutical composition in unit dose form of claim 11, that is comprised in a capsule.

14. A pharmaceutical composition in unit dose form, comprised in a capsule in capsule that comprises a first capsule and a second capsule,
wherein the capsule in capsule contains a first space within the first capsule and a second space located between an outer surface of the first capsule and an inner surface of the second capsule,
wherein the first space contains a first composition comprising:
(i) a first plurality of spray dried particles, wherein each particle of the first plurality of spray dried particles is substantially encapsulated in a coating material and wherein the first plurality of spray dried particles substantially encapsulated in the coating material comprise semaglutide, and
(ii) a phosphatidylcholine (PC);
wherein at least a portion of the first plurality of spray dried particles substantially encapsulated in the coating material individually have a particle diameter ranging from about 10 micrometers to about 200 micrometers, as measured by a particle analyzer using laser diffraction; and
the coating material comprises a hydroxypropyl methylcellulose acetate succinate (HPMCAS); and
the second space contains a second composition comprising
(iii) a second plurality of spray dried particles, wherein each particle of the second plurality of spray dried particles is substantially encapsulated in a coating material and wherein the second plurality of spray dried particles substantially encapsulated in the coating material comprise semaglutide, and
(iv) a phosphatidylcholine (PC);
wherein at least a portion of the second plurality of spray dried particles substantially encapsulated in the coating material individually have a particle diameter ranging from about 10 micrometers to about 200 micrometers, as measured by a particle analyzer using laser diffraction; and
the coating material comprises a hydroxypropyl methylcellulose acetate succinate (HPMCAS).

15. The pharmaceutical composition in unit dose form of claim 15, wherein the first composition comprises from about 5 mg to about 20 mg of the semaglutide; and the second composition independently comprises from about 5 mg to about 20 mg of the semaglutide.

16. The pharmaceutical composition in unit dose form of claim 15, wherein in the first composition, a weight to weight ratio of the semaglutide to the phosphatidylcholine (PC) is about: 1:1. 1:2, 1:3, 1:4, 1:5, or 1:6; and in the second composition, a weight to weight ratio of the semaglutide to the phosphatidylcholine (PC) is independently about 1:1. 1:2, 1:3, 1:4, 1:5, or 1:6.

17. The pharmaceutical composition in unit dose form of claim 15, wherein the first composition comprises about 14 mg of the semaglutide.

18. The pharmaceutical composition in unit dose form of claim 16, wherein in the first composition a weight to weight ratio of the semaglutide to the phosphatidylcholine (PC) is about 1:3.

19. The pharmaceutical composition in unit dose form of claim 17, wherein in the first composition a weight to weight ratio of the semaglutide to the phosphatidylcholine (PC) is about 1:3.

20. A method of treating a disease in a human subject in need thereof, the method comprising orally administering a therapeutically effective amount of the pharmaceutical composition in unit dose form of claim 13 to the human subject in need thereof, thereby treating the disease, wherein the disease is an obesity, a type 1 diabetes, a type 2 diabetes, a type 3c diabetes, an overweight, a coronary artery disease (CAD), an ischemic heart disease (IHD), a myocardial ischemia, or any combination thereof.
